(19) United States
(12) Patent Application Publication
(10) Pub. No.: US 2014/0235705 A1
(43) Pub. Date: Aug. 21, 2014

(54) FORMULATIONS OF THIOPHENE COMPOUNDS

(71) Applicant: Vertex Pharmaceuticals Incorporated, Boston, MA (US)

(72) Inventors: Nicholas Cardoso, East Providence, RI (US); Chong-Hui Gu, Waban, MA (US); Peter A. Ojakovo, Boston, MA (US); Tapan Sanghvi, Watertown, MA (US); Eric A. Simone, Newbury, MA (US); Marinus J. Verwijs, Framingham, MA (US)

(73) Assignee: Vertex Pharmaceuticals Incorporated, Boston, MA (US)

(21) Appl. No.: 14/163,064

(22) Filed: Jan. 24, 2014

Related U.S. Application Data
(63) Continuation of application No. PCT/US2012/048272, filed on Jul. 26, 2012.
(60) Provisional application No. 61/511,643, filed on Jul. 26, 2011.
(60) Provisional application No. 61/511,648, filed on Jul. 26, 2011.
(60) Provisional application No. 61/511,647, filed on Jul. 26, 2011.
(60) Provisional application No. 61/545,751, filed on Oct. 11, 2011.
(60) Provisional application No. 61/512,079, filed on Jul. 27, 2011.
(60) Provisional application No. 61/511,644, filed on Jul. 26, 2011.
(60) Provisional application No. 61/623,144, filed on Apr. 12, 2012.

(51) Int. Cl. A61K 31/381

(52) U.S. Cl. CPC A61K 31/381
USPC 514/447; 549/69; 435/184

(57) ABSTRACT

A pharmaceutical composition comprises: a) polymorphic form M or tromethamine salt of Compound (1) represented by the following structural formula:

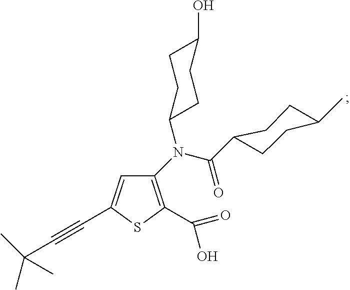 [SMILES: CC1CCC(C(=O)N(c2cc(C#CC(C)(C)C)sc2C(=O)O)C2CCC(O)CC2)CC1]

and b) a filler. A method of preparing a pharmaceutical composition comprises: providing a mixture of Compound (1) and a filler to form the composition of Compound (1). A method of treating a HCV infection in a subject comprises administering to the subject a therapeutically effective amount of the pharmaceutical composition.

### RELATED APPLICATIONS

This application is a continuation of PCT Application No. PCT/US2012/048272, filed Jul. 26, 2012; which in turn claims priority to U.S. Provisional Application No. 61/545,751, filed Oct. 11, 2011; U.S. Provisional Application No. 61/623,144, filed Apr. 12, 2012; U.S. Provisional Application No. 61/511,643, filed Jul. 26, 2011; U.S. Provisional Application No. 61/511,648, filed Jul. 26, 2011; U.S. Provisional Application No. 61/511,647, filed Jul. 26, 2011; U.S. Provisional Application No. 61/512,079, filed Jul. 27, 2011; and U.S. Provisional Application No. 61/511,644, filed Jul. 26, 2011. The entire teachings of these applications are incorporated herein by reference.

### BACKGROUND OF THE INVENTION

Hepatitis C virus (HCV) is a positive-stranded RNA virus belonging to the Flaviviridae family and has closest relationship to the pestiviruses that include hog cholera virus and bovine viral diarrhea virus (BVDV). HCV is believed to replicate through the production of a complementary negative-strand RNA template. Due to the lack of efficient culture replication system for the virus, HCV particles were isolated from pooled human plasma and shown, by electron microscopy, to have a diameter of about 50-60 nm. The HCV genome is a single-stranded, positive-sense RNA of about 9,600 bp coding for a polyprotein of 3009-3030 amino-acids, which is cleaved co and post-translationally into mature viral proteins (core, E1, E2, p′7, NS2, NS3, NS4A, NS4B, NS5A, NS5B). It is believed that the structural glycoproteins, E1 and E2, are embedded into a viral lipid envelope and form stable heterodimers. It is also believed that the structural core protein interacts with the viral RNA genome to form the nucleocapsid. The nonstructural proteins designated NS2 to NS5 include proteins with enzymatic functions involved in virus replication and protein processing including a polymerase, protease and helicase.

The main source of contamination with HCV is blood. The magnitude of the HCV infection as a health problem is illustrated by the prevalence among high-risk groups. For example, 60% to 90% of hemophiliacs and more than 80% of intravenous drug abusers in western countries are chronically infected with HCV. For intravenous drug abusers, the prevalence varies from about 28% to 70% depending on the population studied. The proportion of new HCV infections associated with post-transfusion has been markedly reduced lately due to advances in diagnostic tools used to screen blood donors.

Combination of pegylated interferon plus ribavirin is the treatment of choice for chronic HCV infection. This treatment does not provide sustained viral response (SVR) in a majority of patients infected with the most prevalent genotype (1a and 1b). Furthermore, significant side effects prevent compliance to the current regimen and may require dose reduction or discontinuation in some patients.

Until very recently, the standard of care (SOC) for the treatment of HCV infection comprised 48-week administration of a combination of pegylated interferon-α (subcutaneous weekly injection) and ribavirin (oral, twice daily). Therapy was poorly tolerated and ultimately successful in less than half of the treated patient population. Recently two new treatment regimens for HCV patients have been approved by the FDA that comprise a protease inhibitor (telaprevir or boceprevir) in combination of Peg-IFN/ribavirin. These treatments have demonstrated significantly higher cure rates (sustained viral response (SVR)) in clinical trials in comparison to the then SOC (Peg-IFN/RBV) and are expected to increase treatment success rates (SVR) for HCV patients. There is therefore a great need for the continued development of anti-viral agents and their pharmaceutical compositions for use in treating or preventing Flaviviridae virus infections, such as HCV infections.

### SUMMARY OF THE INVENTION

The present invention generally relates to pharmaceutical compositions that comprise polymorphic Form M or tromethamine salt of Compound (1), to methods of preparing such pharmaceutical compositions, and to methods of treating HCV infections using such pharmaceutical compositions.

In one embodiment, the invention is directed to a pharmaceutical composition comprising: a) polymorphic form M or tromethamine salt of Compound (1) represented by the following structural formula:

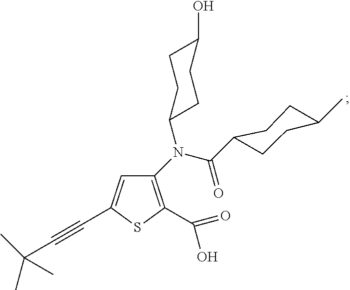 [SMILES: CC1CCC(C(=O)N(c2cc(C#CC(C)(C)C)sc2C(=O)O)C2CCC(O)CC2)CC1]

and b) a filler.

In another embodiment, the invention is directed to a pharmaceutical composition comprising: a) polymorphic form M or tromethamine salt of Compound (1); b) a filler; and c) a disintegrant agent.

In yet another embodiment, the invention is directed to a pharmaceutical composition comprising: a) polymorphic form M or tromethamine salt of Compound (1); b) a wetting agent; c) a binder; d) a disintegrant agent; and e) a filler.

In yet another embodiment, the invention is directed to a pharmaceutical composition comprising: a) 25 wt % to 70 wt % of polymorphic form M or tromethamine salt of Compound (1); and b) 25 wt % to 70 wt % of microcrystalline cellulose, by the weight of the pharmaceutical composition.

In another embodiment, the invention is directed to a pharmaceutical composition comprising: a) 25 wt % to 60 wt % of polymorphic form M or tromethamine salt of Compound (1); b) 0.5 wt % to 10 wt % of polyvinyl pyrrolidone by the weight of the pharmaceutical composition; c) 0.25 wt % to 10 wt % of a copolymer of polyoxypropylene and polyoxyethylene by the weight of the pharmaceutical composition; d) 0.25 wt % to 10 wt % of sodium lauryl sulfate by weight of the pharmaceutical composition; e) 25 wt % to 70 wt % of microcrystalline cellulose by weight of the composition; and f) 1 wt % to 15 wt % of croscarmellose sodium by the weight of the pharmaceutical composition.

In yet another embodiment, the invention is directed to a pharmaceutical composition comprising: a) polymorphic form M or tromethamine salt of Compound (1); b) a complexing agent; and c) a buffering agent.

In yet another embodiment, the invention is directed to a pharmaceutical composition prepared by:

providing granules of Compound (1) that include 35 wt % to 95 wt % of polymorphic form M or tromethamine salt of Compound (1) and 3 wt % to 60 wt % of a filler, by the weight of the granules; and

mixing the granules of Compound (1) with extra-granular excipients that include 10 wt % to 50 wt % of a filler by the weight of the pharmaceutical composition to form the pharmaceutical composition of Compound (1).

In yet another embodiment, the invention is directed to a pharmaceutical composition prepared by:

providing granules of Compound (1) that include 25 wt % to 90 wt % of polymorphic form M or tromethamine salt of Compound (1), and 10 wt % to 25 wt % of a filler, by the weight of the pharmaceutical composition; and

mixing the granules of Compound (1) with extra-granular excipients that include 10 wt % to 50 wt % of a filler by the weight of the pharmaceutical composition, to form the pharmaceutical composition of Compound (1).

In yet another embodiment, the invention is directed to a pharmaceutical composition prepared by:

providing a binder solution that includes 0.5 wt % to 10 wt % of a binder and optionally 0.25 wt % to 10 wt % of a wetting agent, by the weight of the pharmaceutical composition;

providing a pre-granulation composition that includes 25 wt % to 90 wt % of polymorphic form M or tromethamine salt of Compound (1), 10 wt % to 25 wt % of a filler, and 0.5 wt % to 5 wt % of a disintegrant, by the weight of the pharmaceutical composition;

mixing the binder solution and the pre-granulation composition to form a blend; and

mixing the granules of Compound (1) with extra-granular excipients that include 15 wt % to 50 wt % of a filler and 0.5 wt % to 10 wt % of a disintegrant, by the weight of the pharmaceutical composition, to form the pharmaceutical composition of Compound (1),

wherein the mixing of the binder solution and the pre-granulation composition includes feeding the pre-granulation composition into a twin screw extruder and introducing the binder solution into the twin screw extruder.

In yet another embodiment, the invention is directed to a pharmaceutical composition prepared by:

providing a binder solution that includes 0.5 wt % to 10 wt % of a binder and 0.25 wt % to 10 wt % of a wetting agent, by the weight of the pharmaceutical composition;

providing a pre-granulation composition that includes 25 wt % to 60 wt % of polymorphic form M or tromethamine salt of Compound (1), 10 wt % to 25 wt % of a filler, and 0.5 wt % to 5 wt % of a disintegrant, by the weight of the pharmaceutical composition;

mixing the binder solution and the pre-granulation composition to form a blend; and

mixing the granules of Compound (1) with extra-granular excipients that include 15 wt % to 50 wt % of a filler and 0.5 wt % to 10 wt % of a disintegrant, by the weight of the pharmaceutical composition, to form the pharmaceutical composition of Compound (1),

wherein the mixing of the binder solution and the pre-granulation composition includes feeding the pre-granulation composition into a twin screw extruder and introducing the binder solution into the twin screw extruder.

In yet another embodiment, the invention is directed to a method of preparing a pharmaceutical composition, comprising: providing a mixture that includes polymorphic form M or tromethamine salt of Compound (1) and a filler.

In yet another embodiment, the invention is directed to a method of preparing a pharmaceutical composition, comprising: providing a mixture that includes polymorphic form M or tromethamine salt of Compound (1); a filler; and a disintegrant agent.

In yet another embodiment, the invention is directed to a method of preparing a pharmaceutical composition, comprising: providing a mixture that includes polymorphic form M or tromethamine salt of Compound (1), a wetting agent, a binder, a disintegrant agent, and a filler.

In yet another embodiment, the invention is directed to a method of preparing a pharmaceutical composition, comprising: providing a mixture that includes polymorphic form M or tromethamine salt of Compound (1), a complexing agent, and a buffering agent.

In yet another embodiment, the invention is directed to a method of preparing a pharmaceutical composition, comprising: providing granules of Compound (1) that includes polymorphic form M or tromethamine salt of Compound (1), a wetting agent, a binder, and intra-granular excipients that include a filler and a disintegrant agent; and mixing the granules of Compound (1) with extra-granular excipients that include a disintegrant and a filler to form a blended composition of Compound (1).

The invention also provides methods of inhibiting or reducing the activity of HCV polymerase in a biological in vitro sample, comprising administering to the sample an effective amount of a pharmaceutical composition described herein.

Also provided herein are methods of treating a HCV infection in a subject, comprising administering to the subject a therapeutically effective amount of a pharmaceutical composition described herein.

Methods of inhibiting or reducing the activity of HCV polymerase in a subject by employing a therapeutically effective amount of a pharmaceutical composition described herein are also provided.

Use of the pharmaceutical compositions of the invention in the manufacture of a medicament for treating a HCV infection, or inhibiting or reducing the activity of HCV polymerase is also provided.

### BRIEF DESCRIPTION OF DRAWINGS

FIG. 1 shows an XRPD pattern of polymorphic Form A of Compound (1).

FIG. 2 shows a C¹³ solid state NMR spectrum of polymorphic Form A of Compound (1).

FIG. 3 shows an XRPD pattern of polymorphic Form M of Compound (1).

FIG. 4 shows a C¹³ solid state NMR spectrum of polymorphic Form M of Compound (1).

FIG. 5 shows a flow diagram for a wet granulation process for Form M Tablet A

FIG. 6 shows a Form M Tablet A manufacturing flow diagram.

FIG. 7 shows a flow diagram for a wet granulation process for Form M Tablet B.

FIG. 8 shows a flow diagram for blending, compression, and film coating process for Form M Tablet B.

FIG. 9 shows a flow diagram for a wet granulation process for Tablets of Tromethamine Salt of Compound (1)

FIG. 10 shows a tromethamine salt tablet manufacturing flow diagram.

FIG. 11 shows a manufacturing process flow diagram for Drug IV (intra vascular) Solution.

FIG. 12 show dissolution data of Form A capsules, Form M tablets A, Form M tablets B, and tromethamine (Tris) salt tablets in FeSSIF (Fed State Simulated Intestinal Fluid).

FIG. 13 shows the correlation of in vitro z and in vivo z of Compound (1) formulations.

FIG. 14 show dissolution data of Form A capsules, Form M tablets A, Form M tablets B, and tromethamine (Tris) salt tablets in 0.4% SLS (sodium lauryl sulphate).

FIG. 15 shows a diagram of a system used to carry out the instructions encoded by the storage medium of FIGS. 16 and 17.

FIG. 16 shows a cross section of a magnetic storage medium.

FIG. 17 shows a cross section of an optically-readable data storage medium.

FIG. 18 shows a flow diagram for a wet granulation process for Form M Tablet C.

### DETAILED DESCRIPTION OF THE INVENTION

In one aspect, the present invention generally relates to pharmaceutical compositions that comprise polymorphic Form M or tromethamine salt of Compound (1). Compound (1) represented by the following structural formula:

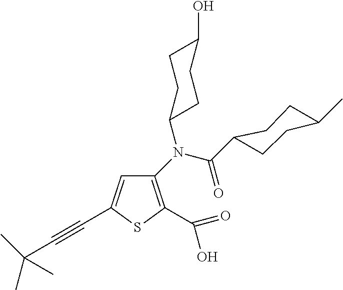 [SMILES: CC1CCC(C(=O)N(c2cc(C#CC(C)(C)C)sc2C(=O)O)C2CCC(O)CC2)CC1]

and pharmaceutically acceptable salts thereof are NS5B polymerase inhibitors, and also described in WO 2008/058393.

Compound (1) can exist in free form, or, where appropriate, as salts. Those salts that are pharmaceutically acceptable are of particular interest since they are useful in administering the compounds described above for medical purposes. Salts that are not pharmaceutically acceptable are useful in manufacturing processes, for isolation and purification purposes, and in some instances, for use in separating stereoisomeric forms of the compounds of the invention or intermediates thereof.

As used herein, the term “pharmaceutically acceptable salt” refers to salts of a compound, which are, within the scope of sound medical judgment, suitable for use in humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. These salts can be prepared in situ during the final isolation and purification of the compounds.

Specific examples of pharmaceutically acceptable salts of Compound (1) are described in WO 2008/058393, such as salts derived from amino acids (e.g. L-arginine, L-Lysine), salts derived from appropriate bases include alkali metals (e.g. sodium, lithium, potassium), alkaline earth metals (e.g. calcium, magnesium), ammonium, NR₄⁺ (where R is C₁₋₄ alkyl) salts, choline and tromethamine salts.

In one embodiment, the present invention employs tromethamine salt of Compound (1).

Compound (1) can also exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds. Generally, different polymorphs can be characterized by analytical methods such as X-ray powder diffraction (XRPD) pattern, thermogravimetric analysis (TGA), and differential scanning calorimetry (DSC), or by its melting point, or other techniques known in the art.

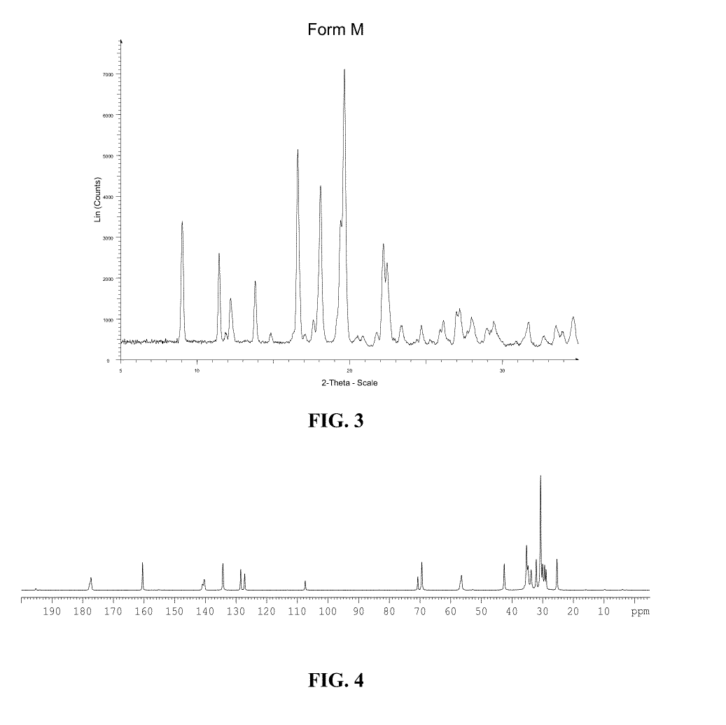

In one embodiment, the present invention employs polymorphic Form M of Compound (1). In one specific embodiment, the polymorphic Form M is characterized as having an X-ray powder diffraction pattern with the most intense characteristic peak expressed in 2-theta±0.2 at 19.6. In another specific embodiment, the polymorphic Form M is characterized as having an X-ray powder diffraction pattern with characteristic peaks expressed in 2-theta±0.2 at the following positions: 19.6, 16.6, 18.1, 9.0, 22.2, and 11.4. In yet another specific embodiment, the polymorphic Form M is characterized as having an X-ray powder diffraction pattern with characteristic peaks expressed in 2-theta±0.2 at the following positions with relative intensities in parentheses: 19.6 (100.0%), 16.6 (72.4%), 18.1 (59.8%), 9.0 (47.6%), 22.2 (39.9%), and 11.4 (36.6%). In yet another specific embodiment, the polymorphic Form M is characterized as having X-ray powder diffraction pattern substantially the same as that shown in FIG. 3. The X-ray powder diffraction patterns are obtained at room temperature using Cu K alpha radiation.

In yet another specific embodiment, the polymorphic Form M is characterized as having an endothermic peak in differential scanning calorimetry (DSC) at 230±2° C. In yet another specific embodiment, the polymorphic Form M is characterized as having peaks at 177.3, 134.3, 107.4, 56.5, 30.7, and 25.3 in a solid state C¹³ nuclear magnetic spectroscopy (NMR) spectrum. In yet another specific embodiment, the polymorphic Form M is characterized as having a solid state C¹³ NMR spectrum substantially the same as that shown in FIG. 4.

Form M of Compound (1) can be prepared by a method employing stirring a mixture of Compound (1) and a solvent system that includes isopropanol, ethyl acetate, n-butyl acetate, methyl acetate, acetone, 2-butanone, or heptane, or a combination thereof at a temperature in a range of 10° C. to 47° C. to form From M of Compound (1). In one specific embodiment, the solvent system includes: isopropanol; ethylacetate; n-butylacetate; a mixture of n-butylacetate and acetone (e.g, 5 wt %-95 wt % of n-butylacetate and 5 wt %-95 wt % of acetone, such as 90 wt % of n-butylacetate and 10 wt % of acetone); a mixture of n-butylacetate and methylacetate (e.g, 5 wt %-95 wt % of n-butylacetate and 5 wt %-95 wt % of methylacetate, such as 50 wt % of n-butylacetate and 50 wt % of methylacetate); acetone; 2-butanone (methylethylketone (MEK)); a mixture of n-butylacetate and heptane (e.g, 5 wt %-95 wt % of n-butylacetate and 5 wt %-95 wt % of heptane, such as 50 wt % of n-butylacetate and 50 wt % of heptane); a mixture of acetone and heptane (e.g, 5 wt %-95 wt % of acetone and 5 wt %-95 wt % of heptane, such as 50 wt % of acetone and 50 wt % of heptane); or a mixture of ethylacetate and heptane (e.g, 5 wt %-95 wt % of ethylacetate and 5 wt %-95 wt % of heptane, such as 50 wt % of ethylacetate and 50 wt % of heptane). In another specific embodiment, Form M of Compound (1) can be prepared by employing stirring Compound (1): i) in isopropanol at a temperature in a range of 10° C. to 47° C.; ii) in ethyl acetate at a temperature in a range of 45° C. to 47° C.; iii) in n-butyl acetate at a temperature in a range of 35° C. to 47° C.; iv) in a mixture of n-butylacetate and acetone (e.g, 5 wt %-95 wt % of butylacetate and 5 wt %-95 wt % of acetone, such as 90 wt % of butylacetate and 10 wt % of acetone) at a temperature in a range of 30° C. to 47° C.; v) in a mixture of n-butylacetate and methylacetate (e.g, 5 wt %-95 wt % of n-butylacetate and 5 wt %-95 wt % of methylacetate, such as 50 wt % of n-butylacetate and 50 wt % of methylacetate) at a temperature in a range of 25° C. to 47° C.; vi) in acetone at a temperature in a range of 20° C. to 47° C.; vii) in 2-butanone (MEK) at a temperature in a range of 30° C. to 47° C.; viii) in a mixture of n-butyl acetate and heptane (e.g, 5 wt %-95 wt % of n-butylacetate and 5 wt %-95 wt % of heptane, such as 50 wt % of n-butylacetate and 50 wt % of heptane) at a temperature in a range of 25° C. to 47° C.; ix) in a mixture of acetone and heptane (e.g, 5 wt %-95 wt % of acetone and 5 wt %-95 wt % of heptane, such as 50 wt % of acetone and 50 wt % of heptane) at a temperature in a range of 25° C. to 47° C.; x) or in a mixture of ethylacetate and heptane (e.g, 5 wt %-95 wt % of ethylacetate and 5 wt %-95 wt % of heptane, such as 50 wt % of ethylacetate and 50 wt % of heptane) at a temperature in a range of 25° C. to 47° C.

Polymorphic From M of Compound (1) described above can be in isolated, pure form, or in a mixture as a solid composition when admixed with other materials, for example the other known polymorphic forms (i.e. amorphous form, Form A of Compound (1), or other forms) of Compound (1) or any other materials. Similarly, the tromethamine salt of Compound (1) can be in isolated, pure form, or in a mixture as a solid composition when admixed with other materials, for example the other known polymorphic forms of Compound (1) or any other materials.

Thus in one aspect there is provided polymorphic Form M or tromethamine salt of Compound (1) in an isolated solid form. In a further aspect there is provided polymorphic Form M or tromethamine salt of Compound (1) in pure form. The pure form means that Form M or tromethamine salt of Compound (1) is over 95% (w/w), for example, over 98% (w/w), over 99% (w/w %), over 99.5% (w/w), or over 99.9% (w/w).

In some embodiments, polymorphic Form M or tromethamine salt of Compound (1) is in the form of a composition or a mixture of the polymorphic form with one or more other crystalline, solvate, amorphous, or other polymorphic forms or their combinations thereof. In one specific embodiment, the composition may comprise polymorphic Form M along with one or more other solid forms of Compound (1), such as amorphous form, hydrate, solvates, polymorph Form A, Form H, Form P, Form X, Form ZA, and/or other forms or their combinations thereof. In another specific embodiment, the composition may comprise the tromethamine salt along with one or more other solid forms of Compound (1), such as amorphous form, hydrate, solvates, polymorph Form A, Form M, Form P, Form X, Form ZA, and/or other forms or their combinations thereof. In yet another specific embodiment, the composition may comprise from trace amounts up to 100% polymorphic Form M of Compound (1), or any amount in between—for example, in a range of 0.1%-0.5%, 0.1%-1%, 0.1%-2%, 0.1%-5%, 0.1%-10%, 0.1%-20%, 0.1%-30%, 0.1%-40%, or 0.1%-50% by weight based on the total amount of Compound (1) in the pharmaceutical composition. In yet another specific embodiment, the composition may comprise at least 50%, 60%, 70%, 80%, 90%, 95%, 97%, 98%, 99%, 99.5% or 99.9% by weight of polymorphic Form M of Compound (1) based on the total amount of Compound (1) in the pharmaceutical composition. In yet another specific embodiment, the composition may comprise from trace amounts up to 100% tromethamine salt of Compound (1), or any amount in between—for example, in a range of 0.1%-0.5%, 0.1%-1%, 0.1%-2%, 0.1%-5%, 0.1%-10%, 0.1%-20%, 0.1%-30%, 0.1%-40%, or 0.1%-50% by weight based on the total amount of Compound (1) in the pharmaceutical composition. In yet another specific embodiment, the composition may comprise at least 50%, 60%, 70%, 80%, 90%, 95%, 97%, 98%, 99%, 99.5% or 99.9% by weight of tromethamine salt of Compound (1) based on the total amount of Compound (1) in the pharmaceutical composition.

In one aspect, the pharmaceutical compositions of the invention comprise polymorphic form M or tromethamine salt of Compound (1) and a filler. In another aspect, the pharmaceutical compositions of the invention comprise polymorphic form M or tromethamine salt of Compound (1), a filler, and disintegrant agent. In yet another aspect, the pharmaceutical compositions of the invention comprise polymorphic form M or tromethamine salt of Compound (1), a binder, a disintegrant agent, and a filler. In yet another aspect, the pharmaceutical compositions of the invention comprise polymorphic form M or tromethamine salt of Compound (1), a wetting agent, a binder, a disintegrant agent, and a filler. Typically, the pharmaceutical compositions include: 25 wt % to 75 wt %, 25 wt % to 70 wt %, or 25 wt % to 60 wt % of the polymorphic form M or tromethamine salt of Compound (1) by weight of the pharmaceutical composition. Typically, the filler comprises 20 wt % to 75 wt %, 20 wt % to 73 wt %, 25 wt % to 75 wt %, 25 wt % to 70 wt %, 30 wt % to 70 wt %, or 35 wt % to 55 wt % of the weight of the pharmaceutical compositions. Typically, the disintegrant agent comprises 1 wt % to 15 wt %, 1 wt % to 10 wt %, 3 wt % to 8 wt %, or 1 wt % to 5 wt % of the pharmaceutical compositions. Typically, the wetting agent comprises 0.25 wt % to 10 wt %, 1 wt % to 10 wt %, or 1 wt % to 5 wt % of the pharmaceutical compositions. Typically, the binder comprises 0.5 wt % to 10 wt %, 1 wt % to 10 wt %, 3 wt % to 8 wt %, or 1 wt % to 5 wt % of the weight of the pharmaceutical compositions. In one embodiment, the pharmaceutical compositions include: 25 wt % to 75 wt % of the polymorphic form M or tromethamine salt of Compound (1) by weight of the pharmaceutical composition; and 20 wt % to 75 wt % (or 20 wt % to 73 wt %) of a filler by weight of the pharmaceutical composition. In another embodiment, the pharmaceutical compositions include: 25 wt % to 70 wt % of the polymorphic form M or tromethamine salt of Compound (1) by weight of the pharmaceutical composition; and 25 wt % to 70 wt % of a filler by weight of the pharmaceutical composition. In another embodiment, the pharmaceutical compositions include: 25 wt % to 70 wt % of the polymorphic form M or tromethamine salt of Compound (1) by weight of the pharmaceutical composition; 25 wt % to 70 wt % of a filler by weight of the composition; and 1 wt % to 15 wt % of a disintegrant agent by weight of the pharmaceutical composition. In another embodiment, the pharmaceutical compositions include: 25 wt % to 70 wt % of the polymorphic form M or tromethamine salt of Compound (1) by weight of the pharmaceutical composition; 25 wt % to 73 wt % of a filler by weight of the composition; and 1 wt % to 15 wt % of a disintegrant agent by weight of the pharmaceutical composition. In yet another embodiment, the pharmaceutical compositions include: 25 wt % to 70 wt % of the polymorphic form M or tromethamine salt of Compound (1) by weight of the pharmaceutical composition; 0.5 wt % to 10 wt % of a binder by weight of the pharmaceutical composition; 1 wt % to 15 wt % of a disintegrant agent by weight of the pharmaceutical composition; and 25 wt % to 70 wt % (or 25 wt % to 73 wt %) of a filler by weight of the pharmaceutical composition. In yet another embodiment, the pharmaceutical compositions include: 25 wt % to 60 wt % of the polymorphic form M or tromethamine salt of Compound (1) by weight of the composition; 0.25 wt % to 10 wt % of a wetting agent by weight of the pharmaceutical composition; 0.5 wt % to 10 wt % of a binder by weight of the composition; 1 wt % to 15 wt % of a disintegrant agent by weight of the pharmaceutical composition; and 25 wt % to 70 wt % (or 25 wt % to 73 wt %) of a filler by weight of the pharmaceutical composition. In yet another embodiment, the pharmaceutical compositions include: 25 wt % to 60 wt % of the polymorphic form M or tromethamine salt of Compound (1) by weight of the pharmaceutical composition; 1 wt % to 5 wt % of a wetting agent by weight of the pharmaceutical composition; 1 wt % to 5 wt % of a binder by weight of the pharmaceutical composition; 1 wt % to 5 wt % of a disintegrant agent by weight of the pharmaceutical composition; and 30 wt % to 70 wt % of a filler (or 25 wt % to 73 wt %) by weight of the pharmaceutical composition. In yet another example, the pharmaceutical compositions include: 25 wt % to 60 wt % of the polymorphic form M or tromethamine salt of Compound (1) by weight of the pharmaceutical composition; 1 wt % to 5 wt % of a wetting agent by weight of the pharmaceutical composition; 1 wt % to 5 wt % of a binder by weight of the pharmaceutical composition; 1 wt % to 5 wt % of a disintegrant agent by weight of the pharmaceutical composition; and 35 wt % to 55 wt % of a filler by weight of the pharmaceutical composition. The wetting agents, binders, disintegrants, and fillers suitable for the invention are compatible with the ingredients of the pharmaceutical compositions of the invention—for example, they do not substantially reduce the chemical stability.

Wetting agents typically include surfactants, such as non-ionic surfactants and anionic surfactants. Wetting agents suitable for the present invention generally enhance the solubility of pharmaceutical compositions. Exemplary surfactants include sodium lauryl sulfate (SLS), polyoxyethylene sorbitan fatty acids (e.g., TWEEN™), sorbitan fatty acid esters (e.g., Spans®), sodium dodecylbenzene sulfonate (SDBS), dioctyl sodium sulfosuccinate (Docusate), dioxycholic acid sodium salt (DOSS), Sorbitan Monostearate, Sorbitan Tristearate, Sodium N-lauroylsarcosine, Sodium Oleate, Sodium Myristate, Sodium Stearate, Sodium Palmitate, Gelucire 44/14, ethylenediamine tetraacetic acid (EDTA), Vitamin E d-alpha tocopheryl polyethylene glycol 1000 succinate (TPGS), Lecithin, MW 677-692, Glutanic acid monosodium monohydrate, Labrasol, PEG 8 caprylic/capric glycerides, Transcutol, diethylene glycol monoethyl ether, Solutol HS-15, polyethylene glycol/hydroxystearate, Taurocholic Acid, copolymers of polyoxypropylene and polyoxyethylene (e.g., poloxamers also known and commercially available under Pluronics®, such as, Pluronic® L61, Pluronic® F68, Pluronic® F108, and Pluronic® F127), saturated polyglycolized glycerides (Gelucirs®), and any combinations thereof. Specific examples include sodium lauryl sulfate, which is an anionic surfactant; and copolymers of polyoxypropylene and polyoxyethylene which are non-ionic surfactants. Specific examples of the copolymers of polyoxypropylene and polyoxyethylene include poloxamers, such as poloxamer with a polyoxypropylene molecular mass of 1,800 g/mol and a 80% polyoxyethylene content (e.g., poloxamer 188). Typical amounts of the wetting agents relative to the total weight of the pharmaceutical composition may be 0.25 wt % to 10 wt %, or 1 wt % to 5 wt %.

Binders typically include agents used while making granules of the active ingredient by mixing it with diluent fillers. Exemplary binders include a polyvinyl pyrrolidone, pregelatinized starch, starch, microcrystalline cellulose, and modified cellulose (e.g., hydroxylpropyl methyl cellulose (HPMC), hydroxypropyl cellulose (HPC) and hydroxy ethyl cellulose (HEC)), and any combinations thereof. Specific examples of the binders include polyvinyl pyrrolidones (PVP). An example of HPC includes a low viscosity polymer, HPC-SL. PVP is commonly characterized by the so-called “K-value,” which is a useful measure of the polymeric composition's viscosity. PVP can be commercially purchased (e.g., Tokyo Chemical Industry Co., Ltd.) under the trade name of Povidone® K12, Povidone® K17, Povidone® K25, Povidone® K30, Povidone® K60, and Povidone® K90. Specific examples of PVP include soluble spray dried PVP. A more specific example includes PVP having an average molecular weight of 3,000 to 4,000, such as Povidone® K12 having an average molecular weight of 4,000. PVP can be used in either wet or dry state. Typical amounts of the binders relative to the total weight of the pharmaceutical composition may be 0.5 wt % to 10 wt %, or 1 wt % to 5 wt %.

Fillers (or diluents) typically include microcrystalline celluloses (e.g., Avicel® PH 101), lactoses, sorbitols, celluoses, calcium phosphates, starches, sugars (e.g., mannitol, sucrose, or the like), or any combination thereof. Specific examples of the fillers include microcrystalline celluloses and lactoses. Specific examples of microcrystalline celluloses include commercially available Avicel® series, such as microcrystalline celluloses having a particle size of 200 mesh over 70% and a particle size of 65 mesh less then 10% (e.g., Avicel® PH 101). A specific example of lactose suitable for the invention includes lactose monohydrate. Typical amounts of the fillers relative to the total weight of the pharmaceutical composition may be 20 wt % to 75 wt %, 20 wt % to 73 wt %, 25 wt % to 75 wt %, 25 wt % to 70 wt %, 30 wt % to 70 wt %, or 35 wt % to 55 wt %.

Disintegrants typically enhance the dispersal of pharmaceutical compositions. Examples of disintegrants include croscarmellose sodium, starch (e.g., corn starch, potato starch), sodium starch glycolate, crospovidone, and any combinations thereof. Specific examples of disintegrants include croscarmellose sodium (e.g., Ac-Di-Sol®) and sodium starch glycolate. Typical amounts of the disintegrants relative to the total weight of the pharmaceutical composition may be 1 wt % to 15 wt %, 1 wt % to 10 wt %, 3 wt % to 8 wt %, or 1 wt % to 5 wt % of the pharmaceutical compositions.

In one embodiment, the pharmaceutical compositions of the invention comprise 25 wt % to 60 wt % of polymorphic form M or tromethamine salt of Compound (1) by weight of the pharmaceutical composition; 0.5 wt % to 10 wt % of polyvinyl pyrrolidone by weight of the composition; 0.25 wt % to 10 wt % of a copolymer of polyoxypropylene and polyoxyethylene by weight of the pharmaceutical composition; 0.25 wt % to 10 wt % of sodium lauryl sulfate by weight of the composition; 25 wt % to 70 wt % of microcrystalline cellulose by weight of the pharmaceutical composition; and 1 wt % to 15 wt % of croscarmellose sodium by weight of the pharmaceutical composition. In a specific embodiment, the pharmaceutical compositions of the invention comprise 25 wt % to 60 wt % of polymorphic form M or tromethamine salt of Compound (1) by weight of the pharmaceutical composition; 1 wt % to 5 wt % of polyvinyl pyrrolidone by weight of the pharmaceutical composition; 1 wt % to 5 wt % of a copolymer of polyoxypropylene and polyoxyethylene by weight of the pharmaceutical composition; 1 wt % to 5 wt % of sodium lauryl sulfate by weight of the pharmaceutical composition; 30 wt % to 70 wt % of microcrystalline cellulose by weight of the pharmaceutical composition; and 1 wt % to 5 wt % of croscarmellose sodium by weight of the pharmaceutical composition. In another specific embodiment, the pharmaceutical compositions of the invention comprise: 25 wt % to 60 wt % of polymorphic form M or tromethamine salt of Compound (1) by weight of the composition; 1 wt % to 5 wt % of polyvinyl pyrrolidone by weight of the pharmaceutical composition; 1 wt % to 5 wt % of a copolymer of polyoxypropylene and polyoxyethylene by weight of the pharmaceutical composition; 1 wt % to 5 wt % of sodium lauryl sulfate by weight of the pharmaceutical composition; 35 wt % to 55 wt % of microcrystalline cellulose by weight of the pharmaceutical composition; and 1 wt % to 5 wt % of croscarmellose sodium by weight of the pharmaceutical composition. In another specific embodiment, the pharmaceutical compositions of the invention comprise: 25 wt % to 70 wt % of polymorphic form M or tromethamine salt of Compound (1) by weight of the pharmaceutical composition; 1 wt % to 5 wt % of polyvinyl pyrrolidone by weight of the pharmaceutical composition; 1 wt % to 5 wt % of a copolymer of polyoxypropylene and polyoxyethylene by weight of the pharmaceutical composition; 1 wt % to 5 wt % of sodium lauryl sulfate by weight of the pharmaceutical composition; 35 wt % to 55 wt % of microcrystalline cellulose by weight of the pharmaceutical composition; and 1 wt % to 5 wt % of croscarmellose sodium by weight of the pharmaceutical composition. In yet another specific embodiment, the pharmaceutical compositions of the invention comprise: 25 wt % to 70 wt % of polymorphic form M or tromethamine salt of Compound (1) by the weight of the pharmaceutical composition; 0.5 wt % to 10 wt % of a polyvinyl pyrrolidone by the weight of the pharmaceutical composition; 0.25 wt % to 5 wt % of a copolymer of polyoxypropylene and polyoxyethylene by the weight of the pharmaceutical composition; 0.25 wt % to 5 wt % of sodium lauryl sulfate by the weight of the pharmaceutical composition; 0.25 wt % to 5 wt % of sodium stearyl fumarate by the weight of the pharmaceutical composition; 20 wt % to 60 wt % (or 20 wt % to 55 wt %) of a microcrystalline cellulose by the weight of the pharmaceutical composition; 0.5 wt % to 15 wt % (or 0.5 wt % to 10 wt %) of a lactose by the weight of the pharmaceutical composition; and 1 wt % to 10 wt % of croscarmellose sodium by the weight of the pharmaceutical composition. In yet another specific embodiment, the pharmaceutical compositions of the invention comprise: 25 wt % to 70 wt % of polymorphic form M or tromethamine salt of Compound (1) by the weight of the pharmaceutical composition; 0.5 wt % to 10 wt % of a hydroxylpropyl cellulose by the weight of the pharmaceutical composition; 0.25 wt % to 10 wt % of sodium stearyl fumarate (or magnesium stearate) by the weight of the pharmaceutical composition; 20 wt % to 60 wt % (or 20 wt % to 55 wt %) of a microcrystalline cellulose by the weight of the pharmaceutical composition; 0.5 wt % to 15 wt % (or 0.5 wt % to 10 wt %) of a lactose by the weight of the pharmaceutical composition; and 1 wt % to 15 wt % of croscarmellose sodium by the weight of the pharmaceutical composition. In yet another specific embodiment, the pharmaceutical compositions of the invention comprise: 25 wt % to 70 wt % of polymorphic form M or tromethamine salt of Compound (1) by the weight of the pharmaceutical composition; 0.25 wt % to 10 wt % of magnesium stearate (or sodium stearyl fumarate) by the weight of the pharmaceutical composition; 25 wt % to 70 wt % of a microcrystalline cellulose by the weight of the pharmaceutical composition; and 1 wt % to 15 wt % of croscarmellose sodium by the weight of the pharmaceutical composition.

Specifically, the polyvinyl pyrolidone has an average molecular weight of 3,000 to 4,000, such as Povidone® K12 having an average molecular weight of 4,000. Specifically, the copolymer of polyoxypropylene and polyoxyethylene is a poloxamer, such as poloxamer with a polyoxypropylene molecular mass of 1,800 g/mol and 80% polyoxyethylene content (e.g., Poloxamer 188). Specifically, the hydroxylpropyl cellulose is a water soluble polymer. In particular, the hydroxylpropyl cellulose is a low-viscosity polymer, such as HPC-SL. Specifically, the microcrystalline cellulose has a particle size of 200 mesh over 70% and a particle size of 65 mesh less then 10%, such as Avicel® PH 101.

In some embodiments, the pharmaceutical compositions of the invention further employ a flow aid or glidant. Typically, glidants enhance the flow properties of pharmaceutical compositions. Exemplary glidants include colloidal silicon dioxide, talc, and a combination thereof. A specific example of glidants includes amorphous, colloidal silicon dioxide having an average particle size in 0.2-0.3 microns, such as Cab-O-Sil® M5P. Typical amounts of the glidants relative to the total weight of the pharmaceutical composition may be 0.1 wt % to 3 wt %, or 0.1 wt % to 1 wt %.

In some embodiments, the pharmaceutical compositions of the invention further employ a lubricant. Lubricants typically improve the compression and ejection of pharmaceutical compositions from, e.g., a die press. Exemplary lubricants include magnesium stearate, stearic acid (stearin), hydrogenated oil, sodium stearyl fumarate, and any combinations thereof. A specific example of the lubricants includes sodium stearyl fumarate. Another specific example of the lubricants includes magnesium stearate. Typical amounts of the lubricants relative to the total weight of the pharmaceutical composition may be 0.25 wt % to 5 wt % or 1 wt % to 5 wt %.

In another aspect, the pharmaceutical compositions of the invention are IV formulations that comprise polymorphic form M or tromethamine salt of Compound (1), a complexing agent, and a buffering agent. Typically, the pharmaceutical compositions include: 1 mg/mL to 20 mg/mL of polymorphic form M or tromethamine salt of Compound (1); 1 wt % to 25 wt % of complexing agent by weight of the pharmaceutical composition; and 0.01 M to 0.1 M of buffering agent. More typically, the pharmaceutical compositions include: 1 mg/mL to 15 mg/mL of polymorphic form M or tromethamine salt of Compound (1); 1 wt % to 25 wt % of complexing agent by weight of the pharmaceutical composition; and 0.01 M to 0.1 M or 0.05 M to 0.1 M of buffering agent. More typically, the pharmaceutical compositions include: 1 mg/mL to 10 mg/mL of polymorphic form M or tromethamine salt of Compound (1); 1 wt % to 25 wt % of complexing agent by weight of the pharmaceutical composition; and 0.01 M to 0.1 M or 0.05 M to 0.1 M of buffering agent. Typical complexing agents include cyclodextrins. More typical complexing agents include sulf-butyl-beta-cyclodextrin and hydroxypropyl-beta-cyclodextrin. Typical buffering agents include monobasic sodium phosphate and dibasic sodium phosphate.

In some embodiments, the IV formulations further comprise dextrose and/or manitol as tonicity modifiers.

In some embodiments, the pharmaceutical compositions of the invention further comprise a colorant, such as Opadry II white.

In some embodiments, the pharmaceutical compositions of the invention are in solid dosage forms, specifically in tablet forms.

Methods of preparing the pharmaceutical compositions described above are also encompassed in the invention. In one embodiment, the methods employ providing a mixture that includes polymorphic form M or tromethamine salt of Compound (1) and a filler to form the pharmaceutical compositions. In another embodiment, the methods employ providing a mixture that includes polymorphic form M or tromethamine salt of Compound (1), a wetting agent, a binder, a disintegrant agent, and a filler to form the pharmaceutical compositions. Typical examples, including specific examples, of the wetting agents, binders, disintegrant agents, and fillers are each and independently as described above.

In yet another embodiment, the methods employ mixing polymorphic form M or tromethamine salt of Compound (1), a complexing agent, and a buffering agent. Specific examples of the complexing agent and buffering agent are each and independently as described above.

In yet another embodiment, the methods employ providing granules of Compound (1) that include polymorphic form M or tromethamine salt of Compound (1) and intra-granular excipients that include a filler; and mixing the granules of Compound (1) with extra-granular excipients that include a filler to form a blended composition of Compound (1). In some specific embodiments, the intra- and extra-granular excipients independently further include a disintegrant agent.

In yet another embodiment, the methods employ: i) providing granules of Compound (1) that include polymorphic form M or tromethamine salt of Compound (1); a binder; intra-granular excipients that include a filler and a disintegrant agent; and optionally a wetting agent, and ii) mixing the granules of Compound (1) with extra-granular excipients that include a disintegrant and a filler to form a blended composition of Compound (1). Typically, the intragranular excipients include 10 wt % to 25 wt % of a filler and 0.5 wt % to 5 wt % of a disintegrant, by the weight of the pharmaceutical composition, and the extra-granular excipients include 15 wt % to 50 wt % of a filler and 0.5 wt % to 10 wt % of a disintegrant by the weight of the pharmaceutical composition. Alternatively, the intragranular excipients include 3 wt % to 50 wt % (or 5 wt % to 50 wt % or 10 wt % to 25 wt %) of a filler and 0.5 wt % to 10 wt % (or 0.5 wt % to 5 wt %) of a disintegrant, by the weight of the granules, and the extra-granular excipients include 15 wt % to 50 wt % of a filler and 0.5 wt % to 10 wt % of a disintegrant by the weight of the pharmaceutical composition. Typical examples, including specific examples, of the wetting agents, binders, disintegrant agents, and fillers are each and independently as described above.

In a specific embodiment, the preparation of granules of Compound (1) includes: providing a binder solution that includes the binder and the wetting agent; providing a pre-granulation composition that includes a polymorphic form M or tromethamine salt of Compound (1) and the intra-granular excipients; adding the binder solution to the pre-granulation composition to form granules of Compound (1). Specifically, the mixing of the binder solution and the pre-granulation composition includes feeding the pre-granulation composition into a twin screw extruder and introducing the binder solution into the extruder. Typically, the intragranular excipients include 3 wt % to 50 wt % (or 5 wt % to 50 wt % or 10 wt % to 25 wt %) of a filler and 0.5 wt % to 10 wt % (or 0.5 wt % to 5 wt %) of a disintegrant, by the weight of the granules, and the extra-granular excipients include 15 wt % to 50 wt % of a filler and 0.5 wt % to 10 wt % of a disintegrant by the weight of the pharmaceutical composition. Typically, the binder solution includes 0.5 wt % to 10 wt % of binder and 0.25 wt % to 10 wt % of wetting agent, by the weight of the pharmaceutical composition. In some specific embodiments, the binder solution further includes water in a range of 5 wt % to 60 wt %, 5 wt % to 30 wt %, or 5 wt % to 15 wt %, by the weight of the pharmaceutical composition.

In another specific embodiment, the methods employ twin screw wet granulation of polymorphic form M or tromethamine salt of Compound (1). In one further specific embodiment, the methods employ: providing granules of Compound (1) that include 35 wt % to 95 wt % of polymorphic form M or tromethamine salt of Compound (1) and 3 wt % to 60 wt % of a filler, by the weight of the granules; and mixing the granules of Compound (1) with extra-granular excipients that include 10 wt % to 50 wt % of a filler by the weight of the pharmaceutical composition to form the pharmaceutical composition of Compound (1). In another further specific embodiment, the methods employ: providing granules of Compound (1) that include: i) 40 wt % to 90 wt % of a polymorphic form M or tromethamine salt of Compound (1); and ii) an intra-granular excipient that includes 5 wt % to 50 wt % of a filler, by the weight of the granules; and mixing the granules of Compound (1) with extra-granular excipients that include 15 wt % to 50 wt % of a filler by the weight of the pharmaceutical composition. In yet another further specific embodiment, the granules of Compound (1) include 25 wt % to 90 wt % of polymorphic form M or tromethamine salt of Compound (1) and 10 wt % to 50 wt % of a filler, by the weight of the granules.

In yet another further specific embodiment, the methods employ: providing a binder solution that includes 0.5 wt % to 10 wt % of a binder and optionally 0.25 wt % to 10 wt % of a wetting agent, by the weight of the pharmaceutical composition; providing a pre-granulation composition that includes 25 wt % to 90 wt % of polymorphic form M or tromethamine salt of Compound (1), 2 wt % to 40 wt % of a filler, and 0.5 wt % to 8 wt % of a disintegrant, by the weight of the pharmaceutical composition; mixing the binder solution and the pre-granulation composition to form granules of Compound (1); and mixing the granules of Compound (1) with extra-granular excipients that include 15 wt % to 50 wt % of a filler and 0.5 wt % to 10 wt % of a disintegrant, by the weight of the pharmaceutical composition, to form a blended composition of Compound (1), wherein the mixing of the binder solution and the pre-granulation composition includes feeding the pre-granulation composition into a twin screw extruder and introducing the binder solution into the twin screw extruder. In yet another further specific embodiment, the methods employ: providing a binder solution that includes 0.5 wt % to 10 wt % of a binder and optionally 0.25 wt % to 10 wt % of a wetting agent, by the weight of the pharmaceutical composition; providing a pre-granulation composition that includes 25 wt % to 75 wt % of polymorphic form M or tromethamine salt of Compound (1), 10 wt % to 25 wt % of a filler, and 0.5 wt % to 5 wt % of a disintegrant, by the weight of the pharmaceutical composition; mixing the binder solution and the pre-granulation composition to form granules of Compound (1); and mixing the granules of Compound (1) with extra-granular excipients that include 15 wt % to 50 wt % of a filler and 0.5 wt % to 10 wt % of a disintegrant, by the weight of the pharmaceutical composition, to form a blended composition of Compound (1), wherein the mixing of the binder solution and the pre-granulation composition includes feeding the pre-granulation composition into a twin screw extruder and introducing the binder solution into the twin screw extruder. In some specific embodiments, the pre-granulation composition includes 25 wt % to 90 wt %, 25 wt % to 75 wt %, 25 wt % to 60 wt %, or 25 wt % to 55 wt %) of polymorphic form M or tromethamine salt of Compound (1). In some specific embodiments, the pre-granulation composition includes: 10 wt % to 25 wt % of a filler, and 0.5 wt % to 5 wt % of a disintegrant, by the weight of the pharmaceutical composition.

In yet another specific embodiment, the methods employ high shear wet granulation. Specifically, the methods employ: providing a granulation composition that includes 25 wt % to 90 wt % 25 wt % to 75 wt % or 25 wt % to 90 wt % of polymorphic form M or tromethamine salt of Compound (1) by the weight of the pharmaceutical composition, 20 wt % to 75 wt % (or 25 wt % to 75 wt % or 20 wt % to 60 wt %) of filler by the weight of the pharmaceutical composition, 0.5 wt % to 10 wt % of binder by the weight of the pharmaceutical composition, and 1 wt % to 10 wt % of disintegrant by the weight of the pharmaceutical composition, and optionally 0.25 wt % to 10 wt % of wetting agent by the weight of the pharmaceutical composition; high shear wet granulating (e.g., using a high shear granulator) in the presence of 10 wt % to 30 wt % water relative to the total weight of the granulation composition to form granules of Compound (1). The granules of Compound (1) are milled and the milled granules are mixed with a filler and a disintegrant, and optionally further with a lubricant. Typically, 60 wt % to 80 wt % of the milled granules of Compound (1) are mixed with 10 wt % to 30 wt % of filler and 1 wt % to 15 wt % of disintegrant, and optionally further with 0.25 wt % to 5 wt % of lubricant.

For tablet compositions of the invention, the methods further comprise film coating the tablet compositions. Typical film coating materials include one or more colorants, such as Opadry II white.

Dissolution of the pharmaceutical compositions of the invention can be estimated with, for example, a standard USP Type II apparatus that employs a suitable dissolution media. Examples of suitable dissolution media include 0.4% sodium lauryl sulphate dissolved in 900 mL of DI water and buffered at pH 4.8 using citrate buffer, and FeSSIF (Fed State Simulated Intestinal Fluid), exemplified in Journal of Pharmacy and Pharmacology, 56, 453-462 (2004), the entire contents of which are hereby incorporated by reference. Typically, the dissolution is measured under stirring at 50-75 rpm at a temperature of approximately 37° C. In some specific embodiments, 50% or more of the pharmaceutical compositions of the invention dissolve within 30 minutes.

As described below, dissolution of the pharmaceutical compositions of the invention can be also measured with the dissolution constant, z, in accordance with Equation (1):

$$\frac{d M}{d t} = z \times {{M}_{0} ( \frac{{M}_{0} - M}{{M}_{0}} )}^{\frac{2}{3}} \times ( {C}_{s} - \frac{M}{V} ) ,$$

where M is a dissolved mass of an active drug, M₀ is the initial mass of the active drug, t is the dissolution time, Cₛ is the solubility of the drug in the dissolution medium, V is the volume of dissolution medium, and z is the dissolution rate constant. z represents the mass transfer property of the drug in a given medium. See Takano, et al., “Oral Absorption of Poorly Water-Soluble Drugs: Computer Simulation of Fraction Absorbed in Humans from a Miniscale Dissolution Test,” Pharmaceutical Research, 23(6), 1144 (2006). In one embodiment, a dissolution rate factor z of a pharmaceutical composition disclosed herein in a simulated human intestinal medium (e.g., FeSSIF) is at least 0.025 ml/mg/min. In another embodiment, the dissolution rate factor z is in a range of 0.025 ml/mg/min to 100 ml/mg/min in a simulated human intestinal fluid. In yet another embodiment, the dissolution rate factor z is in a range of 10 ml/mg/min to 100 ml/mg/min in a simulated human intestinal fluid.

Simulated human intestinal media suitable for the invention can be found in the art, for example, those described in Jantratid, et al., “Dissolution Media Simulating Conditions in the Proximal Human Gastrointestinal Tract: An Update,” Pharmaceutical Research, 25(7), 2008. In one embodiment, a human fed state simulated intestinal fluid (FESSIF) is employed in the invention. A typical FESSIF includes sodium hydroxide, citric acid, sodium chloride, sodium taurocholate, and lecithin (L-Alpha-Phosphatidyl Choline). A specific example is described in Example 9 below.

The solubility (Cₛ) of Compound (1) in a dissolution medium can be determined by any suitable method known in the art. In one embodiment, the solubility of Compound (1) in FESSIF is as follows: 0.9 mg/mL for Form A of Compound (1); 0.825 mg/mL for Form M of Compound (1); 2.6 mg/mL for tromethamine salt of Compound (1).

In some embodiments, the invention is directed to methods of generating an in vivo dissolution profile of an active drug, such as Compound (1). The methods typically include: a) providing a z-factor correlation between in vivo and in vitro z values of Equation (1) using an in vitro dissolution profile (e.g., dissolution of an active drug versus time) and an in vivo dissolution profile (e.g., a concentration of an active drug in plasma versus time) of a first pharmaceutical composition that comprises an active drug and a pharmaceutically acceptable carrier or excipient; b) providing an in vitro z value of Equation (1) using an in vitro dissolution profile of a second pharmaceutical composition that comprises an active drug and a pharmaceutically acceptable carrier or excipient; and c) generating an in vivo dissolution profile of the second pharmaceutical composition using the z-factor correlation and the in vitro dissolution profile of the second pharmaceutical composition. The z-factor correlation can be generated using an in vitro dissolution profile and an in vivo dissolution profile of the first pharmaceutical composition. Specifically, from the in vitro and in vivo dissolution profiles of the first pharmaceutical composition, in vitro and in vivo z values of Equation (1) can be obtained and a z-factor correlation between the in vitro and in vivo z values (e.g., in vitro z value versus in vivo z value) can be generated. Typically, in vivo and in vitro z values can be estimated from in vivo and in vitro dissolution profiles using any suitable fitting program or model known in the art. For example, in vitro z values can be obtained by fitting the in vitro dissolution profile using Mathematica® software, and in vivo z values can be estimated from an in vivo dissolution profile using a biopharmaceutical model known in the art (e.g., . . . , “Predicting the Impact of Physiological and Biochemical Processes on Oral Drug Bioavailability,” Adv. Drug Delivery Review, 50(Suppl. 1), 2001, S41-67). A biopharmaceutical model typically is built to describe a human plasma concentration versus time profile of an active drug using, for example, Gastroplus™ software. The biopharmaceutical model generally contains an advanced compartment and transition (ACAT) model to describe dissolution in the gastrointestinal tract and oral absorption, and a 2-compartment pharmacokinetics model. In general, the in vitro dissolution profiles can be obtained in a suitable dissolution medium such as a simulated human intestinal medium described above (e.g., human FESSIF). In some specific embodiments, the methods include: a) providing an in vitro and in vivo dissolution profiles of a first pharmaceutical composition that comprises an active drug and a pharmaceutically acceptable carrier or excipient; b) providing an in vivo z value of Equation (1) using the in vivo dissolution profile, and an in vitro z value of Equation (1) using the in vitro dissolution profile; c) generating a z-factor correlation between the in vivo and in vitro z values obtained in step b); d) generating an in vitro dissolution profile of a second pharmaceutical composition comprising an active drug and a pharmaceutically acceptable carrier or excipient; e) providing an in vitro z value of Equation (1) using the in vitro dissolution profile of the second pharmaceutical composition; and f) generating an in vivo dissolution profile of the second pharmaceutical composition using the z-factor correlation and the in vitro dissolution profile of the second pharmaceutical composition. Herein, the term “providing” include producing or generating graphs, computer outputs, etc. Without being bound to a particular theory, these methods can expedite the developmental processes of pharmaceutically acceptable compositions of an active drug, such as Compound (1) without obtaining an in vivo (e.g., human plasma) dissolution profile of each pharmaceutically acceptable composition under development.

In another aspect, the invention provides computer systems for the above-described methods of generating an in vivo dissolution profile of an active drug. In one embodiment, the computer systems include a data storage medium that comprises a data storage material encoded with machine-readable data. In one specific embodiment, the data comprises in vivo and in vitro z values of Equation (1). In another specific embodiment, the data comprises in vivo and in vitro z values of Equation (1), and in vivo and in vitro dissolution profiles of a first pharmaceutical composition of an active drug. In yet another specific embodiment, the data comprises in vivo and in vitro z values of Equation (1), in vivo and in vitro dissolution profiles of a first pharmaceutical composition of an active drug, and an in vitro dissolution profile of a second pharmaceutical composition of the active drug. Such storage medium encoded with these data when read and utilized by a computer programmed with appropriate software displays, on a computer screen or similar viewing device. In another embodiment, the computer systems further includes: i) a working memory for storing instructions for processing the machine-readable data; ii) a central-processing unit coupled to the working memory and to said machine-readable data storage medium for processing the machine-readable data and generating a z-factor correlation between in vivo and in vitro z values of Equation (1). In one specific embodiment, the central-processing unit further generates an in vitro z value of Equation (1) using an in vitro dissolution profile and/or an in vivo z value of Equation (1) using the respective dissolution profiles.). In another specific embodiment, the central-processing unit further generates in vitro and in vivo z values of Equation (1) using in vitro and in vivo dissolution profiles of a first pharmaceutical composition; generating a z-factor correlation between the in vitro and in vivo z values; and generating an in vitro z value of Equation (1) using an in vitro dissolution profile of a second pharmaceutical composition. In yet another specific embodiment, the central-processing unit further generates an in vivo dissolution profile of a second pharmaceutical composition using the z-factor correlation and the in vitro dissolution profile of the second pharmaceutical composition. In another embodiment, the computer systems further include a display for displaying the z-factor correlation as a graphical representation. In yet another embodiment, the computer systems further include a commercially available software program to display the graphical representation of the z-factor correlation, etc.

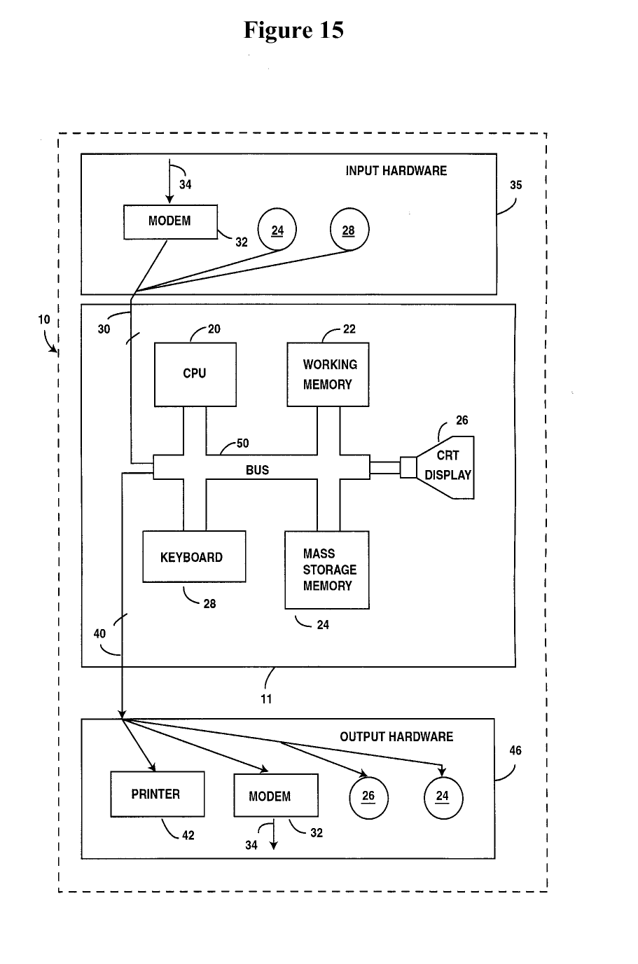

FIG. 15 demonstrates one version of these embodiments. System 10 includes a computer 11 comprising a central processing unit (“CPU”) 20, a working memory 22 which may be, e.g., RAM (random-access memory) or “core” memory, mass storage memory 24 (such as one or more disk drives or CD-ROM drives), one or more cathode-ray tube (“CRT”) display terminals 26, one or more keyboards 28, one or more input lines 30, and one or more output lines 40, all of which are interconnected by a conventional bi-directional system bus 50. Input hardware 36, coupled to computer 11 by input lines 30, may be implemented in a variety of ways. Machine-readable data of this invention may be inputted via the use of a modem or modems 32 connected by a telephone line or dedicated data line 34. Alternatively or additionally, the input hardware 36 may comprise CD-ROM drives or disk drives 24. In conjunction with display terminal 26, keyboard 28 may also be used as an input device. Output hardware 46, coupled to computer 11 by output lines 40, may similarly be implemented by conventional devices. Output hardware might also include a printer 42, so that hard copy output may be produced, or a disk drive 24, to store system output for later use. Output hardware may also include a display terminal, a CD or DVD recorder, ZIP™ or JAZ™ drive, or other machine-readable data storage device. In operation, CPU 20 coordinates the use of the various input and output devices 36, 46, coordinates data accesses from mass storage 24 and accesses to and from working memory 22, and determines the sequence of data processing steps. A number of programs may be used to process the machine-readable data of this invention. Such programs are discussed in reference to the computational methods of drug discovery as described herein. Specific references to components of the hardware system 10 are included as appropriate throughout the following description of the data storage medium.

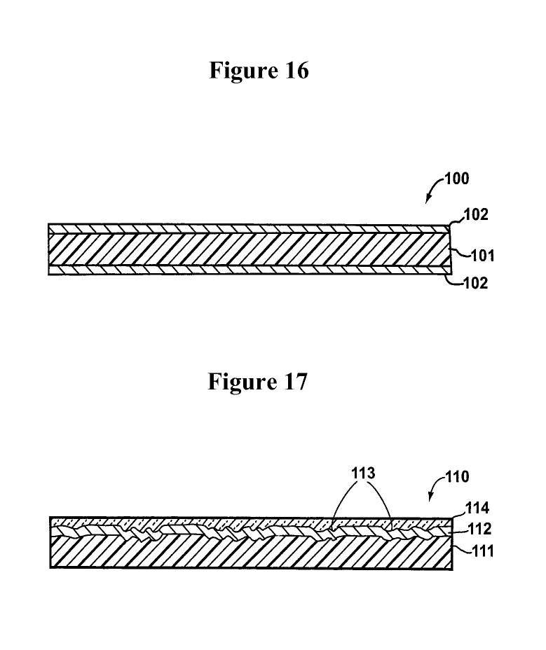

FIG. 16 shows a cross section of a magnetic data storage medium 100 which can be encoded with a machine-readable data that can be carried out by a system such as system 10. Medium 100 can be a conventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device 24. The magnetic domains of coating 102 of medium 100 are polarized or oriented so as to encode in a manner that may be conventional, machine readable data such as that described herein, for execution by a system such as system 10. FIG. 17 shows a cross section of an optically-readable data storage medium 110 which also can be encoded with such a machine-readable data, or set of instructions, which can be carried out by a system such as system 10. Medium 110 can be a conventional compact disk read only memory (CD-ROM) or a rewritable medium such as a magneto-optical disk that is optically readable and magneto-optically writable. Medium 100 preferably has a suitable substrate 111, which may be conventional, and a suitable coating 112, which may be conventional, usually of one side of substrate 111. In the case of CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112. In the case of a magneto-optical disk, as is well known, coating 112 has no pits 113, but has a plurality of magnetic domains whose polarity or orientation can be changed magnetically when heated above a certain temperature, as by a laser (not shown). The orientation of the domains can be read by measuring the polarization of laser light reflected from coating 112. The arrangement of the domains encodes the data as described above.

The pharmaceutical compositions of the invention may further include one or more pharmaceutically acceptable carriers other than those described above. As used herein, “pharmaceutically acceptable” means being inert without unduly inhibiting the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

Some examples of materials which can serve as pharmaceutically acceptable carriers include, but are not limited to, ion exchangers, alumina, aluminum stearate, lecithin, serum proteins (such as human serum albumin), buffer substances (such as phosphates or glycine,), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausolito: 1999, and “March's Advanced Organic Chemistry”, 5th Ed., Ed.: Smith, M. B. and March, J., John Wiley & Sons, New York: 2001, the entire contents of which are hereby incorporated by reference.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this invention, unless only one of the isomers is drawn specifically. As would be understood to one skilled in the art, a substituent can freely rotate around any rotatable bonds. For example, a substituent drawn as

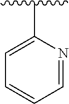 [SMILES: *c1ccccn1]

also represents

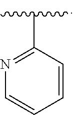 [SMILES: *c1ccccn1]

Therefore, single stereochemical isomers as well as enantiomeric, diastereomeric, cis/trans, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays. Such compounds, especially deuterium (D) analogs, can also be therapeutically useful.

The compounds described herein are defined herein by their chemical structures and/or chemical names. Where a compound is referred to by both a chemical structure and a chemical name, and the chemical structure and chemical name conflict, the chemical structure is determinative of the compound's identity.

It will be appreciated by those skilled in the art that the compounds in accordance with the present invention can exists as stereoisomers (for example, optical (+ and −), geometrical (cis and trans) and conformational isomers (axial and equatorial). All such stereoisomers are included in the scope of the present invention.

It will be appreciated by those skilled in the art that the compounds in accordance with the present invention can contain a chiral center. The compounds of formula may thus exist in the form of two different optical isomers (i.e. (+) or (−) enantiomers). All such enantiomers and mixtures thereof including racemic mixtures are included within the scope of the invention. The single optical isomer or enantiomer can be obtained by method well known in the art, such as chiral HPLC, enzymatic resolution and chiral auxiliary.

In one embodiment, the compounds in accordance with the present invention are provided in the form of a single enantiomer at least 95%, at least 97% and at least 99% free of the corresponding enantiomer.

In a further embodiment, the compounds in accordance with the present invention are in the form of the (+) enantiomer at least 95% free of the corresponding (−) enantiomer.

In a further embodiment, the compounds in accordance with the present invention are in the form of the (+) enantiomer at least 97% free of the corresponding (−) enantiomer.

In a further embodiment, the compounds in accordance with the present invention are in the form of the (+) enantiomer at least 99% free of the corresponding (−) enantiomer.

In a further embodiment, the compounds in accordance with the present invention are in the form of the (−) enantiomer at least 95% free of the corresponding (+) enantiomer.

In a further embodiment, the compounds in accordance with the present invention are in the form of the (−) enantiomer at least 97% free of the corresponding (+) enantiomer.

In a further embodiment the compounds in accordance with the present invention are in the form of the (−) enantiomer at least 99% free of the corresponding (+) enantiomer.

The pharmaceutically acceptable compositions of the invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Specifically, the compositions are administered orally, intraperitoneally or intravenously.

Any orally acceptable dosage form including, but not limited to, capsules, tablets, aqueous suspensions or solutions, can be used for the oral administration. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compound (s) (e.g., polymorphic Form M or tromethamine salt of Compound (1)) can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active compound(s) only, or preferentially, in a certain part of the intestinal tract, optionally, in a delayed manner. Examples of embedding compositions that can be used include polymeric substances and waxes.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in propylene glycol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Sterile injectable forms may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in propylene glycol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In order to prolong the effect of the active compounds administered, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the active compound in biodegradable polymers such as polylactide-polyglycolide. Depending upon the ratio of the active compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

When desired the above described formulations adapted to give sustained release of the active ingredient may be employed.

Compositions for rectal or vaginal administration are specifically suppositories which can be prepared by mixing the active compound with suitable non-irritating excipients or carriers such as cocoa butter, polyethylene glycol or a suppository wax which are solid at ambient temperature but liquid at body temperature and therefore melt in the rectum or vaginal cavity and release the active compound.

Dosage forms for topical or transdermal administration include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, transdermal patches, which have the added advantage of providing controlled delivery of a compound to the body, can also be used. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Alternatively, the active compounds and pharmaceutically acceptable compositions thereof may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, the pharmaceutical compositions of the invention are in solid dosage forms. In some specific embodiments, the pharmaceutical compositions of the invention are in tablet forms. In other embodiments, the pharmaceutical compositions of the invention are in liquid dosage forms, specifically IV dosage forms.

The pharmaceutical compositions of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose. The amount of the active compound in a unit dosage form will vary depending upon, for example, the host treated, and the particular mode of administration, for example, from 0.01 mg/kg body weight/day to 100 mg/kg body weight/day.

It will be appreciated that the amount of the active compounds according to the invention described herein required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 750 mg/kg of body weight per day, for example, in the range of 0.5 to 60 mg/kg/day, or, for example, in the range of 1 to 20 mg/kg/day.

The desired dose may conveniently be presented in a single dose or as divided dose administered at appropriate intervals, for example as two, three, four or more doses per day.

The pharmaceutical compositions of the invention can be used for treating or preventing a Flaviviridae viral infection in a host by administering to the host a therapeutically effective amount of at least one of the active compounds according to the invention described herein.

The terms “subject,” “host,” or “patient” includes an animal and a human (e.g., male or female, for example, a child, an adolescent, or an adult). Preferably, the “subject,” “host,” or “patient” is a human.

In one embodiment, the viral infection is chosen from Flavivirus infections. In one embodiment, the Flavivirus infection is Hepatitis C virus (HCV), bovine viral diarrhea virus (BVDV), hog cholera virus, dengue fever virus, Japanese encephalitis virus or yellow fever virus.

In one embodiment, the Flaviviridea viral infection is hepatitis C viral infection (HCV), such as HCV genotype 1, 2, 3, or 4 infection.

In one embodiment, the active compounds can be used for treatment of HCV genotype 1 infection. The HCV can be genotype 1a or genotype 1b.

In one embodiment, the active compounds can be used for treating or preventing a Flaviviridae viral infection in a host comprising administering to the host a therapeutically effective amount of at least one of the active compounds according to the invention described herein, and further comprising administering at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES).

In one embodiment, there is provided a method for inhibiting or reducing the activity of viral polymerase in a host comprising administering a therapeutically effective amount of the active compounds according to the invention described herein.

In one embodiment, there is provided a method for inhibiting or reducing the activity of viral polymerase in a host comprising administering a therapeutically effective amount of the active compounds according to the invention described herein and further comprising administering one or more viral polymerase inhibitors.

In one embodiment, viral polymerase is a Flaviviridae viral polymerase.

In one embodiment, viral polymerase is a RNA-dependant RNA-polymerase.

In one embodiment, viral polymerase is HCV polymerase.

In one embodiment, viral polymerase is HCV NS5B polymerase.

The pharmaceutical compositions of the invention can be formulated as a pharmaceutical composition which further includes one or more additional agents chosen from viral serine protease inhibitors, viral NS5A inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES). For example, the pharmaceutical composition may include the active compound(s); one or more additional agents select from non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon and ribavirin; and at least one pharmaceutically acceptable carrier or excipient.

One or more additional active agents that can be used as a combination therapy can be chosen from viral serine protease inhibitors, viral polymerase inhibitors, viral NS5A inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

Compound (1) and the additional active agent(s) can be administered sequentially. Alternatively, they can be administered simultaneously. The combinations referred to above may conveniently be presented for use in the form of a pharmaceutical formulation and thus pharmaceutical formulations comprising a combination as defined above together with a pharmaceutically acceptable carrier therefore comprise a further aspect of the invention.

The term “viral serine protease inhibitor” as used herein means an agent that is effective to inhibit the function of the viral serine protease including HCV serine protease in a mammal. Inhibitors of HCV serine protease include, for example, those compounds described in WO 99/07733 (Boehringer Ingelheim), WO 99/07734 (Boehringer Ingelheim), WO 00/09558 (Boehringer Ingelheim), WO 00/09543 (Boehringer Ingelheim), WO 00/59929 (Boehringer Ingelheim), WO 02/060926 (BMS), WO 2006039488 (Vertex), WO 2005077969 (Vertex), WO 2005035525 (Vertex), WO 2005028502 (Vertex) WO 2005007681 (Vertex), WO 2004092162 (Vertex), WO 2004092161 (Vertex), WO 2003035060 (Vertex), of WO 03/087092 (Vertex), WO 02/18369 (Vertex), or WO98/17679 (Vertex).

The term “viral polymerase inhibitors” as used herein means an agent that is effective to inhibit the function of a viral polymerase including an HCV polymerase in a mammal. Inhibitors of HCV polymerase include non-nucleosides, for example, those compounds described in:WO 03/010140 (Boehringer Ingelheim), WO 03/026587 (Bristol Myers Squibb); WO 02/100846 A1, WO 02/100851A2, WO 01/85172 AI (GSK), WO 02/098424 A1 (GSK), WO 00/06529 (Merck), WO 02/06246 A1 (Merck), WO 01/47883 (Japan Tobacco), WO 03/000254 (Japan Tobacco) and EP 1 256 628 A2 (Agouron).

Furthermore other inhibitors of HCV polymerase also include nucleoside analogs, for example, those compounds described in: WO 01/90121A2 (Idenix), WO 02/069903 A2 (Biocryst Pharmaceuticals Inc.), and WO 02/057287 A2 (Merck/Isis) and WO 02/057425 A2 (Merck/lsis).

Specific examples of nucleoside inhibitors of an HCV polymerase, include R1626, R1479 (Roche), R7128 (Roche), MK-0608 (Merck), R1656, (Roche-Pharmasset) and Valopicitabine (Idenix). Specific examples of inhibitors of an HCV polymerase, include JTK-002/003 and JTK-109 (Japan Tobacco), HCV-796 (Viropharma), GS-9190(Gilead), and PF-868,554 (Pfizer).

The term “viral NS5A inhibitor” as used herein means an agent that is effective to inhibit the function of the viral NS5A protease in a mammal. Inhibitors of HCV NS5A include, for example, those compounds described in WO2010/117635, WO2010/117977, WO2010/117704, WO2010/1200621, WO2010/096302, WO2010/017401, WO2009/102633, WO2009/102568, WO2009/102325, WO2009/102318, WO2009020828, WO2009020825, WO2008144380, WO2008/021936, WO2008/021928, WO2008/021927, WO2006/133326, WO2004/014852, WO2004/014313, WO2010/096777, WO2010/065681, WO2010/065668, WO2010/065674, WO2010/062821, WO2010/099527, WO2010/096462, WO2010/091413, WO2010/094077, WO2010/111483, WO2010/120935, WO2010/126967, WO2010/132538, and WO2010/122162. Specific examples of HCV NS5A inhibitors include: EDP-239 (being developed by Enanta); ACH-2928 (being developed by Achillion); PPI-1301 (being developed by Presido Pharmaceuticals); PPI-461 (being developed by Presido Pharmaceuticals); AZD-7295 (being developed by AstraZeneca); GS-5885 (being developed by Gilead); BMS-824393 (being developed by Bristol-Myers Squibb); BMS-790052 (being developed by Bristol-Myers Squibb)

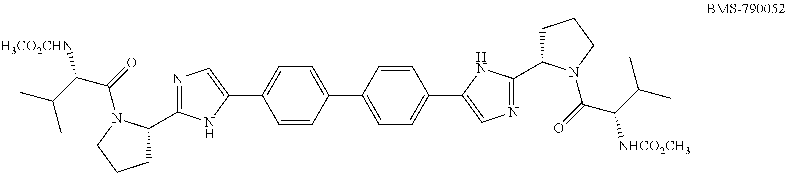

(Gao M. et al. Nature, 465, 96-100 (2010); nucleoside or nucleotide polymerase inhibitors, such as PSI-661 (being developed by Pharmasset), PSI-938 (being developed by Pharmasset), PSI-7977 (being developed by Pharmasset), INX-189 (being developed by Inhibitex), JTK-853 (being developed by Japan Tobacco), TMC-647055 (Tibotec Pharmaceuticals), RO-5303253 (being developed by Hoffmann-La Roche), and IDX-184 (being developed by Idenix Pharmaceuticals).

The term “viral helicase inhibitors” as used herein means an agent that is effective to inhibit the function of a viral helicase including a Flaviviridae helicase in a mammal.

“Immunomodulatory agent” as used herein means those agents that are effective to enhance or potentiate the immune system response in a mammal. Immunomodulatory agents include, for example, class I interferons (such as alpha-, beta-, delta- and omega-interferons, x-interferons, consensus interferons and asialo-interferons), class II interferons (such as gamma-interferons) and pegylated interferons.

Exemplary immunomudulating agents, include, but are not limited to: thalidomide, IL-2, hematopoietins, IMPDH inhibitors, for example Merimepodib (Vertex Pharmaceuticals Inc.), interferon, including natural interferon (such as OMNIFERON, Viragen and SUMIFERON, Sumitomo, a blend of natural interferon's), natural interferon alpha (ALFERON, Hemispherx Biopharma, Inc.), interferon alpha n1 from lymphblastoid cells (WELLFERON, Glaxo Wellcome), oral alpha interferon, Peg-interferon, Peg-interferon alfa 2a (PEGASYS, Roche), recombinant interferon alpha 2a (ROFERON, Roche), inhaled interferon alpha 2b (AERX, Aradigm), Peg-interferon alpha 2b (ALBUFERON, Human Genome Sciences/Novartis, PEGINTRON, Schering), recombinant interferon alfa 2b (INTRON A, Schering), pegylated interferon alfa 2b (PEG-INTRON, Schering, VIRAFERONPEG, Schering), interferon beta-1a (REBIF, Serono, Inc. and Pfizer), consensus interferon alpha (INFERGEN, Valeant Pharmaceutical), interferon gamma-1b (ACTIMMUNE, Intermune, Inc.), un-pegylated interferon alpha, alpha interferon, and its analogs, and synthetic thymosin alpha 1 (ZADAXIN, SciClone Pharmaceuticals Inc.).

The term “class I interferon” as used herein means an interferon selected from a group of interferons that all bind to receptor type 1. This includes both naturally and synthetically produced class I interferons. Examples of class I interferons include alpha-, beta-, delta- and omega-interferons, tau-interferons, consensus interferons and asialo-interferons. The term “class II interferon” as used herein means an interferon selected from a group of interferons that all bind to receptor type II. Examples of class II interferons include gamma-interferons.

Antisense agents include, for example, ISIS-14803.

Specific examples of inhibitors of HCV NS3 protease, include BILN-2061 (Boehringer Ingelheim) SCH-6 and SCH-503034/Boceprevir(Schering-Plough), VX-950/telaprevir(Vertex) and ITMN-B (InterMune), GS9132 (Gilead), TMC-435350(Tibotec/Medivir), ITMN-191 (InterMune), MK-7009 (Merck).

Inhibitor internal ribosome entry site (IRES) includes ISIS-14803 (ISIS Pharmaceuticals) and those compounds described in WO 2006019831 (PTC therapeutics).

In one embodiment, the additional active agents for the compositions and combinations include, for example, ribavirin, amantadine, merimepodib, Levovirin, Viramidine, and maxamine.

In one embodiment, the additional active agent is interferon alpha, ribavirin, silybum marianum, interleukine-12, amantadine, ribozyme, thymosin, N-acetyl cysteine or cyclosporin.

In one embodiment, the additional active agent is interferon alpha 1A, interferon alpha 1 B, interferon alpha 2A, or interferon alpha 2B. Interferon is available in pegylated and non pegylated forms. Pegylated interferons include PEGASYS™ and Peg-intron™.

The recommended dose of PEGASYS™ monotherapy for chronic hepatitis C is 180 mg (1.0 mL vial or 0.5 mL prefilled syringe) once weekly for 48 weeks by subcutaneous administration in the abdomen or thigh.

The recommended dose of PEGASYS™ when used in combination with ribavirin for chronic hepatitis C is 180 mg (1.0 mL vial or 0.5 mL prefilled syringe) once weekly.

Ribavirin is typically administered orally, and tablet forms of ribavirin are currently commercially available. General standard, daily dose of ribavirin tablets (e.g., about 200 mg tablets) is about 800 mg to about 1200 mg. For example, ribavirn tablets are administered at about 1000 mg for subjects weighing less than 75 kg, or at about 1200 mg for subjects weighing more than or equal to 75 kg. Nevertheless, nothing herein limits the methods or combinations of this invention to any specific dosage forms or regime. Typically, ribavirin can be dosed according to the dosage regimens described in its commercial product labels.

The recommended dose of PEG-lntron™ regimen is 1.0 mg/kg/week subcutaneously for one year. The dose should be administered on the same day of the week.

When administered in combination with ribavirin, the recommended dose of PEG-lntron is 1.5 micrograms/kg/week.

The combinations referred to above may conveniently be presented for use in the form of a pharmaceutical formulation and thus pharmaceutical formulations comprising a combination as defined above together with a pharmaceutically acceptable carrier therefore comprise a further aspect of the invention. The individual components for use in the method of the present invention or combinations of the present invention may be administered either sequentially or simultaneously in separate or combined pharmaceutical formulations.

In one embodiment, the additional agent is interferon α 1A, interferon α 1B, interferon α 2A, or interferon α 2B, and optionally ribavirin.

When Compound (1) is used in combination with at least one second therapeutic agent active against the same virus, the dose of each compound may be either the same as or differ from that when the compound is used alone. Appropriate doses will be readily appreciated by those skilled in the art.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXEMPLIFICATION

### Example 1

### General Methods of XRPD and C¹³ Solid State NMR Measurements

SSNMR Experimental:

Solid state nuclear magnetic spectroscopy (SSNMR) spectra were acquired on Bruker 400 MHz proton frequency wide bore spectrometer. Form A was acquired on Bruker 500 MHz spectrometer. Before obtaining carbon spectra, proton relaxation longitudinal relaxation times (¹H T₁) were determined by fitting proton detected proton saturation recovery data to an exponential function. These values were used to set an optimal recycle delay of carbon cross-polarization magic angle spinning experiment (¹³C CPMAS), which, typically, was set between 1.2×¹H T₁ and 1.5×¹H T₁. The carbon spectra were acquired with 2 ms contact time using linear amplitude ramp on proton channel (from 50% to 100%) and 100 kHz SPINAL-64 decoupling. The typical magic angle spinning (MAS) speed was 12.5 kHz. To limit a frictional heating due to fast spinning, the probe temperature was maintained at 275K. Carbon spectra were referenced externally by setting the upfield resonance of solid phase sample of adamantane to 29.5 ppm. Using this procedure, carbon spectra were indirectly referenced to tetramethyl silane at 0 ppm.

Bruker D8 Discover XRPD Experimental Details.

The XRPD patterns were acquired at room temperature in reflection mode using a Bruker D8 Discover diffractometer (Asset Tag V012842) equipped with a sealed tube source and a Hi-Star area detector (Bruker AXS, Madison, Wis.). The X-Ray generator was operating at a voltage of 40 kV and a current of 35 mA. The powder sample was placed in an aluminum holder. Two frames were registered with an exposure time of 120 s each. The data were subsequently integrated over the range of 4°-40° 2θ with a step size of 0.02° and merged into one continuous pattern.

### Example 2

### Formation of Compound (1)

### Method A

Compound (1) can be prepared as described in WO 2008/058393:

### Preparation of 5-(3,3-Dimethyl-but-1-ynyl)-3-[(trans-4-hydroxy-cyclohexyl)-(trans-4-methyl-cyclohexanecarbonyl)-amino]-thiophene-2-carboxylic acid

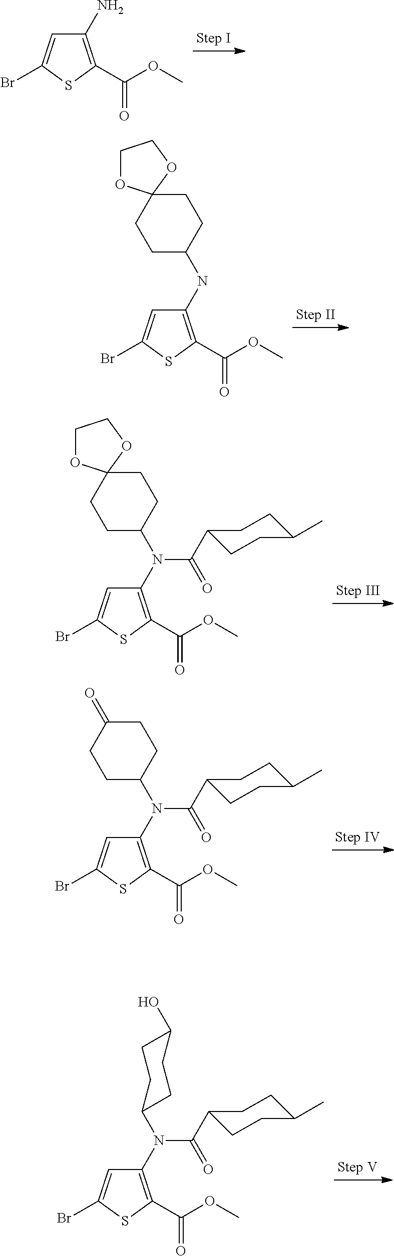

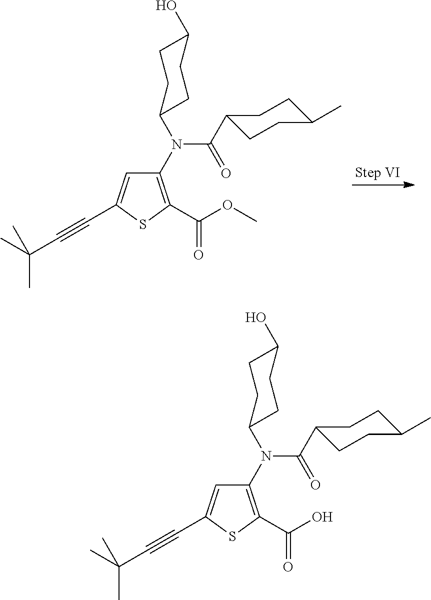

Step I

A suspension of 3-amino-5-bromo-thiophene-2-carboxylic acid methyl ester (17.0 g, 72.0 mmol) in dry THF (21 mL) is treated with 1,4-cyclohexanedione monoethylene ketal (11.3 mg, 72.0 mmol), followed by dibutyltin dichloride (1.098 g, 3.6 mmol). After 5 min, phenyl silane (9.74 mL, 79.2 mmol) is added and the reaction mixture is stirred over-night at room temperature. After concentration, the residue is dissolved in EtOAc washed with NaHCO₃ then brine. The organic layer is separated, dried on Na₂SO₄, filtered and concentrated. The crude material is diluted in hexane (500 mL). After filtration, the mother liquor is evaporated to dryness to give 5-bromo-3-(1,4-dioxa-spiro[4.5]dec-8-ylamino)-thiophene-2-carboxylic acid methyl ester (24.79 g, 92% yield).

### Ref: WO2004/052885

Step II

A—Preparation of trans-4-methylcyclohexyl carboxylic acid chloride:

Oxalyl chloride (2M in DCM, 117 mL) is added drop wise to a suspension of trans-4-methylcyclohexyl carboxylic acid (16.6 g, 117 mmol) in DCM (33 mL) and DMF (0.1 mL), and the reaction mixture is stirred 3 h at room temperature. DCM is removed under reduced pressure and the residue is co-evaporated with DCM. The residue is dissolved in toluene to make a 1M solution.

B—Preparation of the Target Compound:

The 1M solution of trans-4-methylcyclohexyl carboxylic acid chloride is added to a solution of 5-bromo-3-(1,4-dioxa-spiro[4.5]dec-8-ylamino)-thiophene-2-carboxylic acid methyl ester (24.79 g, 65 mmol) in toluene (25 mL) followed by pyridine (5.78 mL, 71.5 mmol). The resulting mixture is then stirred for 16 h at reflux. The reaction mixture is diluted with toluene (60 mL) and cooled down to 5° C. After the addition of pyridine (12 mL) and MeOH (5.6 mL), the mixture is stirred 2 h at 5° C. The white suspension is filtered off and the toluene is added to the mother liquor. The organic phase is washed with 10% citric acid, aq. Sat NaHCO₃, dried (Na₂SO₄) and concentrated. The residue is triturated in boiling hexane (1500 mL). The reaction mixture is allowed to cool down to room temperature. The reaction flask is immersed into ice bath, and stirred for 30 min; white solid is filtered off, and washed with cold hexane (225 mL). The solid is purified by silica gel column chromatography using 20% EtOAc:hexane as eluent to furnish the final compound 5-bromo-3-[(1,4-dioxa-spiro[4.5]dec-8-yl)-(trans-4-methyl-cyclohexanecarbonyl)-amino]-thiophene-2-carboxylic acid methyl ester (10.5 g, 32%).

Step III

5-Bromo-3-[(1,4-dioxa-spiro[4.5]dec-8-yl)-(trans-4-methylcyclohexane-carbonyl)-amino]-thiophene-2-carboxylic acid methyl ester (8.6 g, 17 mmol) is dissolved in tetrahydrofuran (100 mL) and treated with 3N HCl solution (50 mL). The reaction is stirred at 40° C. for 3 h. The reaction mixture is evaporated under reduced pressure. The residue is dissolved in EtOAc and washed with aq. sat. NaHCO₃ solution. The organic layer is separated, dried on Na₂SO₄, filtered and concentrated to give 5-bromo-3-[(trans-4-methyl-cyclohexanecarbonyl)-(4-oxo-cyclohexyl)-amino]-thiophene-2-carboxylic acid methyl ester as a solid (7.4 g, 95%).

Step IV

To a cold (0° C.) solution of 5-bromo-3-[(trans-4-methyl-cyclohexanecarbonyl)-(4-oxo-cyclohexyl)-amino]-thiophene-2-carboxylic acid methyl ester (5.9 g, 12.9 mmol) in 50 mL of MeOH under a N₂ atmosphere, NaBH₄ (250 mg, 6.4 mmol) is added portion wise (approx. 30 min). After the addition is completed and checked for reaction completion by TLC (hexane:EtOAc 1:1), 10 mL of HCl 2% is added and stirred for 15 min. The reaction mixture is concentrated under vacuum to dryness. The reaction mixture is recuperated with water (25 mL) and extracted with EtOAC. The organic phases are combined and dried over MgSO₄ and concentrated to dryness. The residue is purified by silica gel column chromatography using EtOAc:hexane (1:1) as eluent to obtain 5-bromo-3-[(trans-4-hydroxy-cyclohexyl)-(trans-4-methyl-cyclohexane-carbonyl)-amino]-thiophene-2-carboxylic acid methyl ester (4.5 g, 77% yield) as a solid.

Step V

To a solution of compounds 5-bromo-3-[(trans-4-hydroxy-cyclohexyl)-(trans-4-methyl-cyclohexanecarbonyl)-amino]-thiophene-2-carboxylic acid methyl ester (500 mg, 1.09 mmol) and 3,3-Dimethyl-but-1-yne (385 mg, 4.69 mmol) in DMF (0.5 mL), triethylamine (1.06 mL) and tris(dibenzylideneacetone) dipalladium (0) (70 mg, 0.08 mmol) are added and the reaction mixture is stirred under reflux conditions for 16 h under a N₂ atmosphere. DMF and triethylamine are removed under reduced pressure and the residue is partitioned between water and ethyl acetate. The organic layer is separated, dried (Na₂SO₄), concentrated and the residue is purified by column chromatography using ethyl acetate and hexane (1:2) as eluent to obtain 5-(3,3-dimethyl-but-1-ynyl)-3-[(trans-4-hydroxy-cyclohexyl)-(trans-4-methyl-cyclohexanecarbonyl)-amino]-thiophene-2-carboxylic acid methyl ester as a solid, 330 mg (66%).

Step VI

5-(3,3-Dimethyl-but-1-ynyl)-3-[(trans-4-hydroxy-cyclohexyl)-(trans-4-methyl-cyclohexanecarbonyl)-amino]-thiophene-2-carboxylic acid methyl ester (0.10 g, 0.22 mmol) is dissolved in a 3:2:1 mixture of THF:methanol:H₂O (5.0 mL) and treated with a 1N solution of LiOH.H₂O (0.65 mL, 0.65 mmol). After 2 h of stirring at 60° C., the reaction mixture is concentrated under reduced pressure on a rotary evaporator. The mixture is partitioned between ethyl acetate and water. The water layer is acidified using 0.1 N HCl. The EtOAc layer is separated and dried over Na₂SO₄. Filtration and removal of the solvent under reduced pressure on a rotary evaporator followed by purification by column chromatography using methanol and dichloromethane (1:9) as eluent to obtain 5-(3,3-dimethyl-but-1-ynyl)-3-[(trans-4-hydroxy-cyclohexyl)-(trans-4-methyl-cyclohexanecarbonyl)-amino]-thiophene-2-carboxylic acid as a solid, 30 mg (30%). ESI⁻ (M−H): 444.3. ¹H NMR (400 MHz, DMSO-d₆) δ0.58 (m, 1H), 0.74 (q, J=6.53 Hz, 1H), 0.81 (ddd, J=12.86, 12.49, 3.19 Hz, 1H), 1.18 (m, 5H), 1.28 (s, 3H), 1.42 (m, 1H), 1.55 (m, 3H), 1.61 (m, 1H), 1.73 (m, 2H), 1.81 (m, 2H), 3.19 (m, 1H), 4.26 (m, 1H), 4.49 (bs, 1H), 7.14 (s, 1H), 13.45 (bs, 1H).

### Method B

### Preparation of 5-(3,3-Dimethyl-but-1-ynyl)-3-[(trans-4-hydroxy-cyclohexyl)-(trans-4-methyl-cyclohexanecarbonyl)-amino]-thiophene-2-carboxylic acid

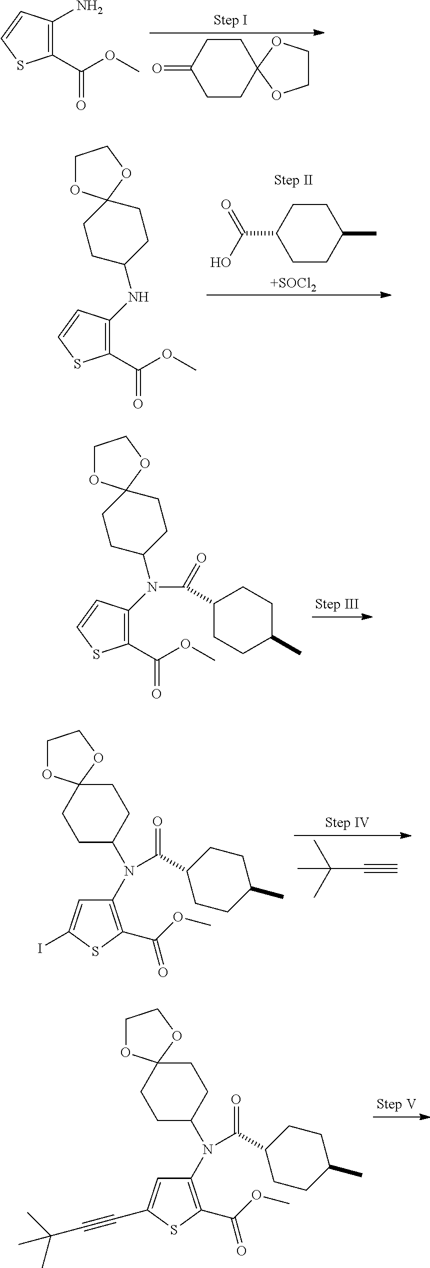

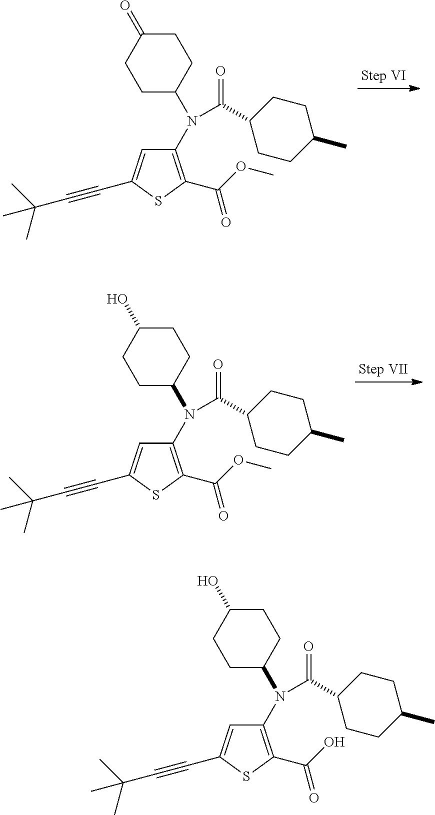

Step 1

A suspension of 3-amino-thiophene-2-carboxylic acid methyl ester (5.0 g, 31.85 mmol) in dry THF (9 mL) is treated with 1,4-cyclohexanedione monoethylene ketal (5.0 g, 32.05 mmol), followed by dibutyltin dichloride (482 mg, 1.59 mmol). After 5 min, phenyl silane (4.3 mL, 34.96 mmol) is added and the reaction mixture is stirred overnight at room temperature. After concentration, the residue is dissolved in EtOAc and washed with NaHCO₃ followed by brine. The organic layer is separated, dried (Na₂SO₄), filtered and concentrated. The residue is purified by column chromatography using 30% ethyl acetate in hexane as eluent to give 3-(1,4-dioxa-spiro[4.5]dec-8-ylamino)-thiophene-2-carboxylic acid methyl ester (4.5 g, 47% yield).

### Alternative Procedure:

3-Amino-thiophene-2-carboxylic acid methyl ester (1 eq.) is dissolved in dichloromethane followed by 1,4-cyclohexanedione monoethylene acetal (2 eq.) to obtain a slightly yellow solution. This solution is added to the suspension of NaBH(OAc)₃ (2.2 eq.) in dichloromethane. Acetic acid (2.4 eq.) is added dropwise over a period of 15 min. The resulting suspension is stirred at 20-25° C. under N₂ for 24 h. The reaction is quenched by adding water and stirred for 1 h. Dichloromethane layer is separated, treated with water again and stirred for another 1 h. The dichloromethane layer is separated and added to a saturated NaHCO₃ solution, stirred at 20-25° C. for 20 min. Some of the white residual solids are filtered and then the organic layer is separated, dried (Na₂SO₄) and evaporated. Methanol is added to the residue and evaporated to dryness. The residue is taken in of methanol and stirred for 2 h at 0° C. The suspension is vacuum-filtered and the resulting filtered cake is washed with cold methanol. The white solid is dried under vacuum at 35-40° C. for approximately 20 h to afford the title compound.

Step II

A. Preparation of trans-4-methylcyclohexyl carboxylic acid chloride

Oxalyl chloride (2M in dichloromethane, 17 mL) is added dropwise to a suspension of trans-4-methylcyclohexyl carboxylic acid (2.3 g, 16.2 mmol) in dichloromethane (5 mL) and DMF (0.1 mL). The reaction mixture is stirred for 3 h at room temperature. The volatiles are removed under reduced pressure to obtain the crude acid chloride which is used directly for the next reaction.

B. trans-4-Methylcyclohexyl carboxylic acid chloride is added to a solution of 3-(1,4-dioxa-spiro[4.5]dec-8-ylamino)-thiophene-2-carboxylic acid methyl ester (2.4 g, 8.08 mmol) in toluene (18 mL) followed by pyridine (0.7 mL). The resulting mixture is then stirred for 16 h at reflux. The reaction mixture is diluted with toluene (7 mL) and cooled to 5° C. After the addition of pyridine (1.5 mL) and MeOH (0.8 mL), the mixture is stirred 2 h at 5° C. The white solid is filtered and washed with toluene. The filtrate is washed with 10% citric acid, aq. NaHCO₃, dried (Na₂SO₄) and concentrated. The solid is purified by silica gel column chromatography using 20% EtOAc:hexane as eluent to obtain 3-[(1,4-dioxa-spiro[4.5]dec-8-yl)-(trans-4-methyl-cyclohexanecarbonyl)-am-ino]-thiophene-2-carboxylic acid methyl ester (2.3 g, 68%).

### Alternative Procedure:

To a solution of trans-4-methylcyclohexyl carboxylic acid (1.8 eq.) in toluene under nitrogen is added anhydrous DMF. The reaction mixture is stirred and thionyl chloride (2.16 eq.) is added over 3-5 min. The mixture is then stirred for 3 h at rt. When the reaction is completed, toluene is added to the reaction mixture. The solution is then evaporated under reduced nitrogen pressure to half of its volume. The solution is dissolved in toluene to obtain a 1N acid chloride solution.

3-(1,4-Dioxa-spiro[4.5]dec-8-ylamino)-thiophene-2-carboxylic acid methyl ester (1 eq.) and pyridine (2 eq.) are added to the acid chloride (1N) solution. The reaction mixture is stirred at reflux for 15 h. Once the reaction is completed, the reaction mixture is cooled to room temperature, and then methanol and toluene are added to it. The reaction mixture is stirred for 1 h at rt and a saturated aqueous solution of NaHCO₃ is added. The organic layer is separated, dried (Na₂SO₄) and evaporated to about 4 volumes of solvent. To the solution are added 4 volumes of heptane while stirring. The reaction flask is immersed into an ice bath and stirred for 120 min; a beige solid is filtered off and washed with cold heptane, then dried over night in the vacuum oven to obtain the title compound.

Step III

n-BuLi (2 eq.) is added dropwise for 10 min to a cold (−40° C.) solution of diisopropylamine (1 eq.) in dry THF. The reaction mixture is stirred at the same temperature for 30 min. Then a solution of 3-[(1,4-dioxa-spiro[4.5]dec-8-yl)-(trans-4-methyl-cyclohexane-carbonyl)-a-mino]-thiophene-2-carboxylic acid methyl ester (1 eq.) in THF is added dropwise (35 min) keeping the internal temperature around −40.degree. C. The reaction mixture is stirred for 30 min and a solution of iodine (2 eq.) in THF is added dropwise, stirred for 30 min at the same temperature before being added a sat. solution of NH₄Cl. The reaction mixture is diluted with ethyl acetate and water. The organic layer is separated and washed with 5% sodium thiosulfate solution. The organic layer is separated, dried (Na₂SO₄) and evaporated to a suspension and then added heptane. The suspension is stirred at 0.degree. C. for 30 min, filtered and washed with heptane to obtain 3-[(1,4-dioxa-spiro[4.5]dec-8-yl)-(trans-4-methyl-cyclo-hexanecarbonyl)-a-mino]-5-iodo-thiophene-2-carboxylic acid methyl ester. MS found (electrospray): (M+H): 548.21

Step IV

To a 25 mL RBF under nitrogen, 3-[(1,4-dioxa-spiro[4.5]dec-8-yl)-(trans-4-methyl-cyclohexanecarbonyl)-am-ino]-5-iodo-thiophene-2-carboxylic acid methyl ester (1 eq.), copper iodide (0.025 eq.) and tris(dibenzylideneacetone) dipalladium (0) (0.01 eq.) are taken. DMF, triethylamine (2.5 eq.) and 3,3-dimethyl-but-1-yne (2 eq.) are added and the reaction mixture is stirred at 40° C. for 2 h under a N₂ atmosphere. The reaction mixture is filtered on celite and washed with ethyl acetate. The solution is diluted with water and extracted 2 times with ethyl acetate. The organic phases are combined and washed 3 times with water. The organic layer is separated, dried (Na₂SO₄), evaporated to about 2 mL and then 8 mL of heptane is added. It is evaporated to 2-4 mL and cooled in an ice bath. The formed white solid is filtered, washed with heptane and dried in oven to obtain 5-(3,3-dimethyl-but-1-ynyl)-3-[(1,4-dioxa-spiro[4.5]dec-8-yl)-(trans-4-me-thyl-cyclohexanecarbonyl)-amino]-thiophene-2-carboxylic acid methyl ester.

Step V

5-(3,3-Dimethyl-but-1-ynyl)-3-[(1,4-dioxa-spiro[4.5]dec-8-yl)-(trans-4-methyl-cyclohexanecarbonyl)-amino]-thiophene-2-carboxylic acid methyl ester (1 eq.) is dissolved in tetrahydrofuran and treated with 3.6 N HCl solution. The reaction is stirred at 40° C. for 5 h. Water is then added and the reaction mixture is cooled to room temperature. The reaction mixture is extracted with ethyl acetate (2.times.50 mL). The combined extracts are washed with 25 mL of aqueous saturated NaHCO₃ and 2×50 mL of water. The organic layer is concentrated to a thick oil and 50 mL of heptane is added to the mixture to precipitate the desired compound which is filtered to give of 5-(3,3-dimethyl-but-1-ynyl)-3-[(trans-4-methyl-cyclohexanecarbonyl)-(4-ox-o-cyclohexyl)-amino]-thiophene-2-carboxylic acid methyl ester.

Step VI

5-(3,3-Dimethyl-but-1-ynyl)-3-[(trans-4-methyl-cyclohexanecarbonyl)-(4-oxo-cyclohexyl)-amino]-thiophene-2-carboxylic acid methyl ester (1 eq.) is dissolved in THF. Water is added to the reaction mixture and cooled to −25.degree. C. NaBH.sub.4 (0.5 eq.) is added portion wise maintaining the temperature below −20.degree. C. The mixture is stirred for 2 h at −25.degree. C., 2N HCl is then added and the solution is warmed to room temperature. The phases are separated and the aqueous layer is washed with EtOAC. The organic phases are combined and washed with brine and dried over Na.sub.2SO.sub.4 and concentrated to dryness to give 5-(3,3-dimethyl-but-1-ynyl)-3-[(4-hydroxy-cyclohexyl)-(trans-4-methyl-cyc-lohexanecarbonyl)-amino]-thiophene-2-carboxylic acid methyl ester as a 93:7 mixture of isomers. The crude cis/trans mixture is recrystallized in methanol to obtain >99% the trans isomer.

Step VII

The same procedure as reported earlier (Method A, step VI) is followed to obtain 5-(3,3-dimethyl-but-1-ynyl)-3-[(trans-4-hydroxy-cyclohexyl)-(trans-4-meth-yl-cyclohexanecarbonyl)-amino]-thiophene-2-carboxylic acid. MS found (electrospray): (M−H): 444.3. ¹H NMR (400 MHz, DMSO-d₆) δ0.58 (m, 1H), 0.74 (q, J=6.53 Hz, 1H), 0.81 (ddd, J=12.86, 12.49, 3.19 Hz, 1H), 1.18 (m, 5H), 1.28 (s, 3H), 1.42 (m, 1H), 1.55 (m, 3H), 1.61 (m, 1H), 1.73 (m, 2H), 1.81 (m, 2H), 3.19 (m, 1H), 4.26 (m, 1H), 4.49 (bs, 1H), 7.14 (s, 1H), 13.45 (bs, 1H).

The methods A and B described above produced methanol solvates of Compound (1) as the final products after the column chromatography using methanol and dichloromethane (1:9) as eluent (step VI of Route A).

### Method C

### 1. Step 1

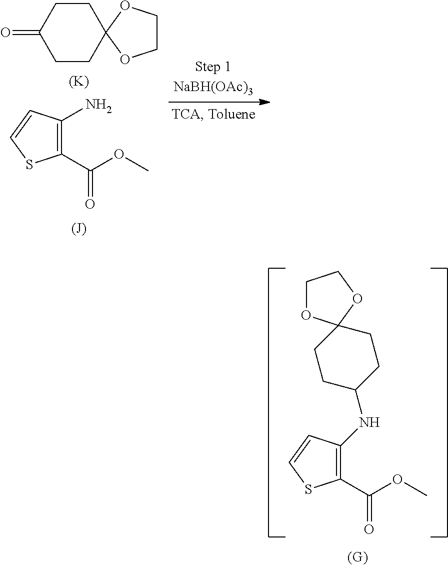

Compounds J (50.0 g, 1.0 eq.), K (52.2 g, 1.05 eq), and NaBH(OAc)₃ (118.0 g, 1.75 eq) were added to a reactor followed by toluene (600 mL, 12 vol). Started agitation then adjusted the internal temperature to 0-5° C. The mixture was a heterogeneous suspension of white solids. Then was added trichloroacetic acid (TCA, 52.0 g, 1.0 eq) in toluene (150 mL, 3 vol) to the stirring mixture over 1 h while controlling the internal temperature to between 0-5° C. The reaction mixture was warmed to 20-25° C., and then stirred for 2-4 hours at 20-25° C. under an atmosphere of nitrogen. The reaction progress was monitored by HPLC.

Upon completion of reaction, the reaction mixture was transferred into a solution of K₂CO₃ (307.7 g, 7.0 eq) in DI water (375 mL, 7.5 vol). The biphasic mixture was stirred and then the phases were separated. The organic phase was washed with aqueous solution of K₂CO₃ (175.9 g, 4.0 eq) in DI water (375 mL, 7.5 vol), then with aqueous solution of NaCl (20.4 g, 1.1 eq) in DI water (375 mL, 7.5 vol). The organic phase was separated. The batch volume was reduced by distillation (to 250 mL (5 vol) on a rotary evaporator at a bath temperature of ≦40° C.) and the resulting crude solution of Compound G in toluene was used in the next step (HPLC: 98.29% AUC chemical purity). Compound G: ¹H NMR (400 MHz, DMSO-d₆) δ 1.45 (m, 2H), 1.64 (m, 4H), 1.88 (m, 2H), 3.56 (m, 1H), 3.72 (s, 3H), 3.87 (m, 4H), 6.70 (d, J=6.8 Hz, 1H), 6.90 (d, J=4.4 Hz, 1H), 7.70 (d, J=4.4 Hz, 1H).

### 2. Step 2

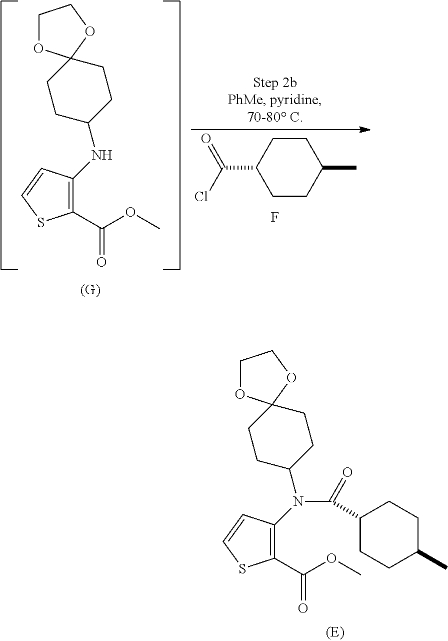

### 2.1. Step 2b: Using trans-methylcyclohexane carbonyl chloride (Compound F)

To the solution of compound G in toluene (94.6 g, 250 mL, 5.0 vol) from previous step was added toluene (410 mL, 8.2 vol) and pyridine (64.0 mL, 2.5 eq). Agitation was started and the internal temperature was adjusted to 20-25° C. Compound F (102.2 g, 2.0 eq) was added over 0.5 h. The batch was heated to 95-100° C. once the addition had complete. The reaction progress was monitored by HPLC. Upon completion of reaction, the batch was cooled to 30-35° C., then methanol (189 mL, 3.8 vol) was added over 45 minutes and the batch was stirred for 1-2 hours. Added DI water (189 L, 3.8 vol) to the bactch at 30-35° C. then it was allowed to stir at 60-70° C. for 1-2 hours. The mixture was heated to 55-60° C. then stirred for 1 h.

The phases were separated. DI water (189 mL, 3.8 vol) was added at 55-60° C. then stirred for 1 hour. The toluene phase was concentrated by distillation. The batch was heated to 78-83° C. (e.g., 80° C.), then n-heptane (473 mL, 9.5 vol) was added to toluene solution over 1-3 hours, and the batch was then stirred at 90-95° C. over 2 hours. The batch was cooled to 20-25° C. over 5 hours, followed by stirring at 20-25° C. for 1-12 hours. The solids were filtered. The filter cake was washed with n-heptane (190 mL, 3.8 vol) and dried under vacuum at 40-45° C. for 10-20 hours. The isolated compound E was analyzed by HPLC, GC, and Karl Fischer titration. Overall yield for Steps 1 & 2=113.5 g, 84.1%. HPLC: 99.39% AUC chemical purity (Typical purity >98.0%). Compound E: ¹H NMR (400 MHz, DMSO-d₆) δ0.48 (m, 1H), 0.63 (m, 1H), 0.74 (d, J=6.4 Hz, 3H), 0.98 (m, 1H), 1.22 (m, 2H), 1.36 (m, 1H), 1.52-1.67 (m, 10H), 1.77 (m, 2H), 3.75-3.78 (m, 4H), 3.76 (s, 3H), 4.44 (m, 1H), 7.11 (d, J=5.2 Hz, 1H), 8.00 (d, J=5.2 Hz, 1H).

### 2.2. Step 2a: Using trans-methylcyclohexane carboxylic acid (Compound H)

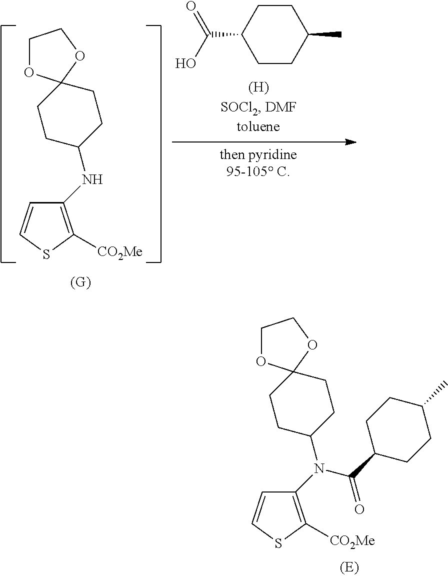

Compound H (633 g, 2.0 eq) was charged to a reactor-1 under a N₂ atmosphere. Toluene (1.33 L, 3.8 vol) was then added to the reactor, followed by DMF (1.73 mL, 0.01 eq), then agitation was started. SOCl₂ (325 mL, 2.0 eq) was added slowly over 30 minutes. The internal temperature was adjusted to 33-37° C. (e.g., 35° C.). The solution was stirred at 33-37° C. for 2 hours. The mixture was cooled to 20-25° C., transferred to a rotary evaporator, and then concentrated to 3.8 vol (˜1.3 L). Toluene (665 mL, 1.9 vol) was then added to the concentrate and the resulting batch was concentrated to 3.8 vol (˜1.3 L).

Compound G in toluene (662 g, 1.75 L, 5.0 vol) was charged to a reactor-2 under N₂ atmosphere. Toluene (4.97 L, 14.2 vol) and pyridine (448 mL, 2.5 eq) was added to the reactor-2. Agitation was started and the internal temperature was adjusted to 20-25° C.

The solution of reactor-1 (acid chloride obtained above) in toluene was added to the reactor-2 over 1 hour. The reaction mixture was heated to 95-105° C. once the addition had complete. An IPC sample was taken after 24-30 h and analyze for Compound G consumption by HPLC.

The reaction mixture was then cooled to 25-30° C. MeOH (665 mL, 1.9 vol) was added to the reaction mixture over 45 minutes. DI water (1.33 L, 3.8 vol) was then added to the reaction mixture at 25-30° C. The mixture was heated to 55-60° C. then stirred for 1 hour. Stopped agitation and allowed the phases to separate for 10 minutes. The upper organic layer was separated and the aqueous layer was set aside. DI water (1.33 L, 3.8 vol) was added to the reaction mixture at 55-60° C. then stirred for 1 hour. Stopped agitation and allowed the phases to separate for 10 minutes. The upper organic layer was separated and the aqueous layer was set aside. The solution was transferred (while it remained at ˜60° C.) to a rotary evaporator and concentrated to 5.7 vol (˜2 L). Heptane (3.3 L, 5.0 vol) was then added to the suspension at ˜60° C. The suspension was cooled to 20-25° C. while stirring over 5 hours. The suspension was filtered. The cake was washed twice with heptane (665 mL, 1.9 vol). The solids were dried on the filter under vacuum. Overall yield for Steps 1 & 2=805.2 g, 85.8% as a white solid. HPLC: 99.15% AUC chemical purity. Compound E: ¹H NMR (400 MHz, DMSO-d₆) δ0.48 (m, 1H), 0.63 (m, 1H), 0.74 (d, J=6.4 Hz, 3H), 0.98 (m, 1H), 1.22 (m, 2H), 1.36 (m, 1H), 1.52-1.67 (m, 10H), 1.77 (m, 2H), 3.75-3.78 (m, 4H), 3.76 (s, 3H), 4.44 (m, 1H), 7.11 (d, J=5.2 Hz, 1H), 8.00 (d, J=5.2 Hz, 1H).

### 3. Step 3

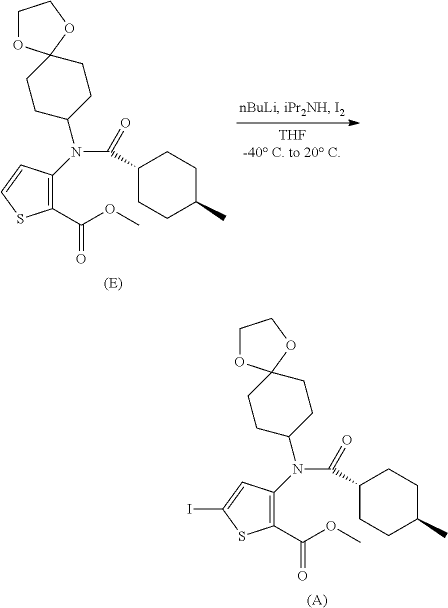

Anhydrous THF (1.0 L, 2.0 vol) and anhydrous diisopropylamine (258 mL, 1.55 eq) were added to Reactor-1. The solution was cooled to −50° C. to −40° C. Once the desired temperature was achieved, a 1.6M solution of n-butyl lithium in hexanes (1.11 L, 1.50 eq) was added at a rate such that the internal temperature remained below −40° C. After the addition had completed, the solution stirred at −50° to −40° C. for another 2 hours.

Compound E (500 g, 1.0 eq) and anhydrous THF (5.0 L, 10.0 vol) were charged to Reactor-2. The resulting solution was added to Reactor-1 over 1 hour at a rate such that the internal temperature remained below −40° C. A solution of iodine (361 g, 1.20 eq) in THF (500 mL, 1.0 vol) was added to the cold reaction mixture at a rate such that the internal temperature remained below −40° C. The reaction mixture was at −50° to −40° C. for 1 hour. The reaction progress was monitored by HPLC.

Upon completion of reaction, the batch was warmed to 0-5° C. and transferred to a solution of NaHSO₃ (617 g, 5.0 eq) in DI water (2.5 L, 5.0 vol) cooled to 0-5° C. Dichloromethane (1.5 L, 3.0 vol) was added to the suspension. The biphasic mixture was stirred for 1 hour while warming to 20-25° C. The phases were separated. The aqueous phase was washed with dichloromethane. The organic phases were combined and washed twice with aqueous solution of NH₄CL (634 g, 10.0 eq) in DI water (1.9 L, 5.0 vol), followed by wash with water. The batch volume was reduced by distillation. Solvent switch to toluene was performed: added toluene (1.5 L, 3.0 vol) again then concentrated to 3.0 vol (˜1.5 L). Toluene (5.0 L, 10.0 vol) was then added to the resulting concentrate and the mixture was heated to 95-100° C. until a homogenous solution was obtained. Added heptane (5.0 L, 10.0 vol) at 95-100° C. to the toluene solution, then the mixture was cooled to 20-25° C. over 6 hours. The suspension was filtered. The cake was washed twice with heptane (500 mL, 1.0 vol). The solids were dried on the filter under vacuum. The isolated compound A was analyzed by HPLC, GC, and Karl Fischer titration. Yield for Steps=520.5 g, 80.2% as a beige solid. HPLC: Typical >97.0% AUC chemical purity. Compound A: ¹H NMR (400 MHz, DMSO-d₆) δ 0.54 (m, 1H), 0.65 (m, 1H), 0.76 (d, J=6.8 Hz, 3H), 1.00 (m, 1H), 1.22 (m, 2H), 1.30 (m, 1H), 1.44-1.68 (m, 10H), 1.60-1.69 (m, 4H), 1.77 (m, 2H), 3.74 (s, 3H), 3.77 (m, 4H), 4.40 (m, 1H), 7.46 (s, 1H).

### 4. Step 4

A. Method A1

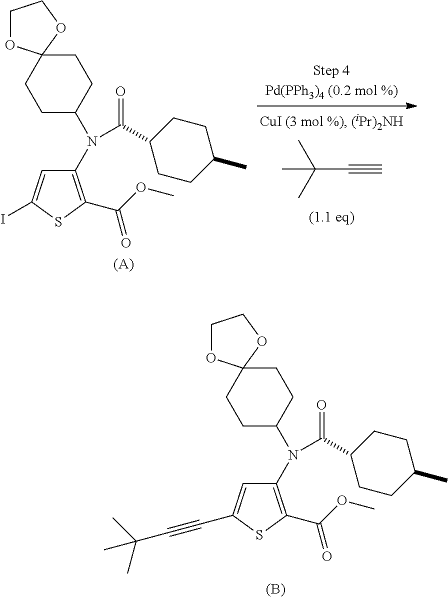

A jacketed 1 L 3-neck reactor was fitted with a nitrogen inlet then charged with Compound (A) (112.7 g, 205.9 mmol). CuI (1.18 g, 6.18 mmol) and Pd(PPh₃)₄ (457.9 mg, 0.412 mmol) were added to the reactor. The reactor was purged with a stream of nitrogen then anhydrous 2-methyltetrahydrofuran (789 mL) was added. The mixture was stirred for 15 mins at 20-25° C. Anhydrous diisopropylamine (52.09 g, 72.15 mL, 514.8 mmol) and tert-butylacetylene (18.59 g, 27.0 mL, 226.5 mmol) were added to the reactor. This mixture was then stirred between 20-25° C. Complete conversion after stirring for 4 h had been reached according to HPLC. The mixture was cooled to 10° C. The organic phase was then washed with 12.6 wt % aqueous oxalic acid for at least 3 hours then the phases were split. Activated carbon (22.5 g) was added to the reaction mixture. The suspension was stirred at 20-25° C. for not less than 12 hours. The mixture was filtered over celite. The filter cake was washed with 2-butanone (563.5 mL) and the filtrate was added to the organic phase. Analysis of the organic solution by HPLC showed Compound (B) purity to be 99.56% AUC. This solution is typically used directly in the next step. Compound (B): ¹H NMR (400 MHz, DMSO-d₆) δ 0.52-0.59 (m, 1H), 0.61-0.70 (m, 1H), 0.76 (d, J=6.4 Hz, 3H), 0.88-1.03 (m, 1H), 1.15-1.37 (m, 4H), 1.31 (s, 9H)S, 1.41-1.68 (m, 9H), 1.74-1.85 (m, 2H), 3.75-3.81 (m, 4H), 3.75 (s, 3H), 4.39-4.42 (m, 1H), 7.27 (s, 1H).

B. Method A2

A jacketed 1 L 3-neck reactor was fitted with a nitrogen inlet then charged with Compound (A) (63.94 g). CuI (667.3 mg, 0.03 eq) and Pd(PPh₃)₄ (269.9 mg, 0.002 eq) were added to the reactor. The reactor was purged with a stream of nitrogen then methyl t-butyl ether (MtBE) (7 vol) was added. The mixture was stirred for 15 mins at 20-25° C. Anhydrous diisopropylamine (40.9 mL, 2.5 eq) was added to the stirring mixture while maintaining the internal temperature between 20-25° C. and stirred the batch for NLT 15 minutes. tert-Butylacetylene (16.7 mL, 1.2 eq) were added to the reactor. This mixture was then stirred between 20-25° C. Complete conversion after stirring for 4 h had been reached according to HPLC. The mixture was cooled to 10° C. The organic phase was then washed with 12.6 wt % aqueous oxalic acid dehydrate (383.6 mL, 6 vol) was added while maintaining the batch temperature below 20-25° C. The batch temperature was then adjusted to 20-25° C. and the biphasic mixture was stirred for at least 3 hours at this temperature. The phases were then allowed to separate for at least 30 minutes. The organic phase was then again washed with aqueous oxalic acid dehydrate (6 wt % m 383.6 mL, 6 vol) while maintaining the batch temperature below 20-25° C. The biphasic mixture was stirred for at least 1 hout at this temperature. Then the phases were split. Activated carbon (6.4 g-12.8 g, 10-20 wt % with respect to Compound A) was added to the reaction mixture. The suspension was stirred at 20-25° C. for not less than 12 hours. The mixture was filtered over celite. The filter cake was washed with MtBE (192 mL, 3 vol) and the filtrate was added to the organic phase. This solution is typically used directly in the next step.

C. Method B

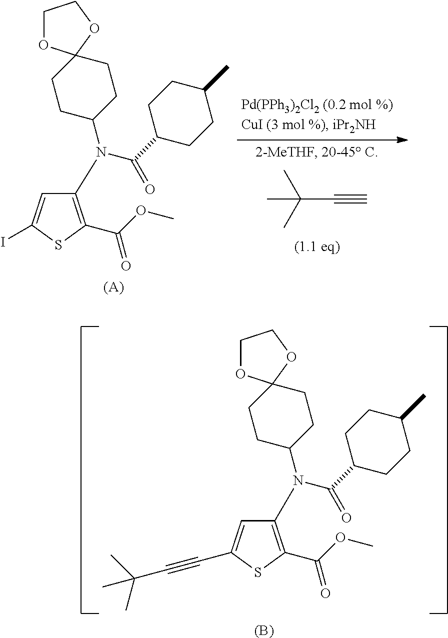

A jacketed 3 L 3-neck reactor was fitted with a nitrogen inlet then charged with Compound (A) (20.00 g, 36.53 mmol). CuI (208.7 mg, 1.096 mmol) and Pd(PPh₃)₂Cl₂ (51.28 mg, 0.07306 mmol) were added to the reactor. The reactor was purged with a stream of nitrogen then anhydrous 2-methyltetrahydrofuran (140.0 mL) was added. The mixture was stirred for 15 mins at 20-25° C. Anhydrous diisopropylamine (9.241 g, 12.80 mL, 91.32 mmol) and tert-butylacetylene (3.751 g, 5.452 mL, 45.66 mmol) were added to the reactor. This mixture was then stirred between 20-25° C. (20.9° C.) (a suspension is formed). The mixture was then heated to 45° C. for 6 h. An HPLC analysis showed conversion to be 99.77%. Heptane (140.0 mL) was added while cooling to 20° C. over 4 h. The suspension was filtered. The filtrate was washed with an aqueous oxalic acid dihydrate solution (120 mL of 15% w/v, 142.8 mmol). The phases were split then the organic phase was washed with aqueous NH₄Cl (120 mL of 10% w/v, 224.3 mmol), aqueous NaHCO₃ (120 mL of 7% w/w), and water (120.0 mL). Residual metals were scavenged by addition of 2.0 g charcoal (10% wt of VRT-0921870) followed by stirring at 20-25° C. for 5 h. The suspension was then filtered over celite. The celite bed was washed with 2-methyltetrahydrofuran (40.0 mL). Analysis of the organic solution by HPLC showed Compound (B) purity to be 99.47% AUC.

D. Method C

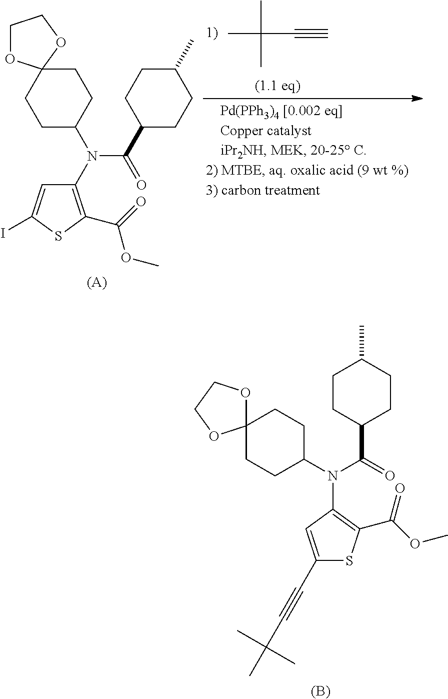

To a round bottom flask equipped with mechanical stirring, N₂ bubbler and thermocouple, was add Compound (A) [1.0 eq], copper catalyst, Pd (PPh₃)₄ [0.002 eq] and MEK [7 volume]. The reaction solution was stirred at room temperature to dissolve followed by addition of iPr₂NH [2.5 equiv] and tert-butylacetylene [1.1 equiv]. The reaction solution was stirred at 20-25° C. The reaction conversion (conv [%]) was monitored via LC. For the copper catalyst, CuI (99.9%), CuI(98%), CuCl, and CuBr were tested:

CuI (for both 99.9% and 98%): with 0.03 equiv of CuI, over 95% conversion into Compound (B) after about 2 hours' reaction time; with 0.025 equiv of CuI, over 90% conversion into Compound (B) after about 5 hours' reaction time; with 0.02 equiv of CuI, over 90% conversion into Compound (B) after about 5 hours' reaction time; with 0.015 equiv of CuI, over 90% conversion into Compound (B) after about 5 hours' reaction time; with 0.01 equiv of CuI, over 75% conversion into Compound (B) after about 5 hours' reaction time;

CuCl: with 0.03 equiv of CuCl, over 99% conversion into Compound (B) after about 2 hours' reaction time; with 0.025 equiv of CuI, approximately 100% conversion into Compound (B) after about 2 hours' reaction time; with 0.02 equiv of CuCl, over 90% conversion into Compound (B) after about 2 hours' reaction time; with 0.015 equiv of CuCl, over 95% conversion into Compound (B) after about 2 hours' reaction time; with 0.01 equiv of CuCl, approximately 100% conversion into Compound (B) after about 20 hours' reaction time;

CuBr: with 0.03 equiv of CuBr, over 99% conversion into Compound (B) after about 22 hours' reaction time; with 0.025 equiv of CuBr, over 85% conversion into Compound (B) after about 22 hours' reaction time; with 0.02 equiv of CuBr, over 95% conversion into Compound (B) after about 22 hours' reaction time; with 0.015 equiv of CuBr, over 70% conversion into Compound (B) after about 22 hours' reaction time; with 0.01 equiv of CuBr, over 80% conversion into Compound (B) after about 22 hours' reaction time.

### 5. Step 5

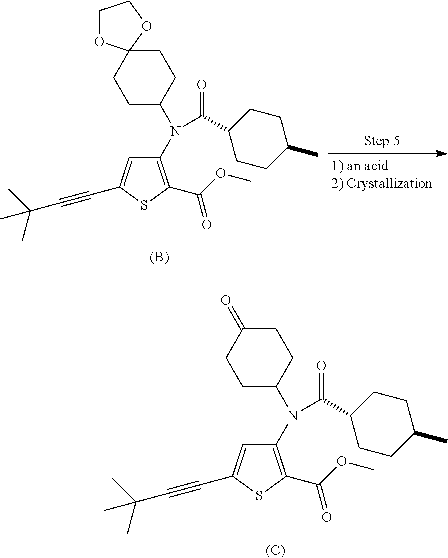

### A. Method A

A jacketed 1 L 4-neck reactor was fitted with a nitrogen inlet then charged with a solution of Compound (B) (22.9 g, 45.65 mmol) in 2-butanone (˜250 mL), then heated to 60° C. The reactor was purged with a stream of nitrogen then an aqueous solution of 2N HCl (175 mL) was added. The mixture was stirred at 60° C. for 4 hours. The stirring was stopped and the lower aqueous phase was removed. Agitation was started again followed by the addition of fresh aqueous solution of 2N HCl (175 mL). The mixture continued to stir at 60° C. until the conversion (99% by HPLC) had reached equilibrium (approximately another 2.5 hours). After cooling to 20° C., the lower aqueous phase was removed. The organic phase was then washed with 10 wt % aqueous NH₄Cl then the phases were split. The organic phase was then distilled to ˜115 mL. Acetone (115 mL) was added then the batch was concentrated to ˜115 mL. This procedure of acetone addition followed by distillation was repeated twice more. Water (57.3 mL) was added to the organic phase at 20° C. then the mixture stirred for 2 hours. Water was added to the organic phase at 20° C. over 2 hours then the mixture stirred for an additional hour. The solids were filtered and washed with 1:1 MeOH/H₂O (25 mL), then dried in a vacuum oven with nitrogen bleed at 60° C. for 24 hours to give 19.8 g (95% yield) of Compound (C). ¹H NMR (400 MHz, DMSO-d₆) δ 0.56-0.68 (m, 2H), 0.76 (d, J=6.4 Hz, 3H), 1.19-1.30 (m, 4H), 1.30 (s, 9H), 1.46-1.60 (m, 6H), 1.83-1.89 (m, 2H), 2.05-2.18 (m, 3H), 2.47-2.55 (m, 1H), 3.76 s, 3H), 4.77-4.85 (m, 1H), 7.30 (s, 1H).

### B. Method B

A jacketed 1 L 4-neck reactor was fitted with a nitrogen inlet then charged with a solution of Compound (B) (103.3 g, 1.0 eq based on 100% yield in Step 4) in 2-butanone (˜1.03 L, approximately 10 vol total batch volume), then heated to 57° C.-62° C. (e.g., 60° C.). The reactor was purged with a stream of nitrogen then an aqueous solution of 2N HCl (723 mL, 7 vol based on 103.3 g of Compound (B)) was added over about 10 minutes while maintaining the batch temperature at 57° C.-62° C. (e.g., 60° C.). The mixture was stirred at 57° C.-62° C. (e.g., 60° C.) for 5 hours. The stirring was stopped and the lower aqueous phase was removed. Agitation was started again followed by the addition of fresh aqueous solution of 2N HCl (310 mL, 3 vol based on 103.3 g of Compound (B)). The mixture continued to stir at 57° C.-62° C. (e.g., 60° C.) until the conversion (99% by HPLC) had reached equilibrium (approximately another 2.5 hours). After cooling to 20-25° C., the agitation was stopped and phases were allowed to separate for at least 30 minutes. An aqueous NH₄Cl (10 wt %, 517 mL, 5 vol) was then added while maintaining the batch temperature at 20-25° C. The biphasic mixture was stirred for at least 30 minutes at 20-25° C. Then the phases were split. The organic phase was then distilled to ˜471 mL by vacuum distillation with a maximum jacketed temperature of 60° C. Acetone (471.1 mL) was added then the batch was concentrated to ˜471 mL. This procedure of acetone addition followed by distillation was repeated twice more. Water (235.6 mL, 2.28 vol) was added to the organic phase at 20° C. then the mixture stirred for 2 hours. Additional water (235.6 mL, 2.28 vol) was added to the organic phase at 20° C. over 2 hours then the mixture stirred for an additional hour. The solids were filtered and washed with a 1:1 mixture of acetaone/H₂O (vol:vol, 103 mL: 103 mL), then dried in a vacuum oven with nitrogen bleed at 60° C. for 24 hours to give 19.8 g (99.5% yield) with overall purity of 98.0%) of Compound (C).

### 6. Step 6

### A. Method A: Using LiAlH(OtBu)₃

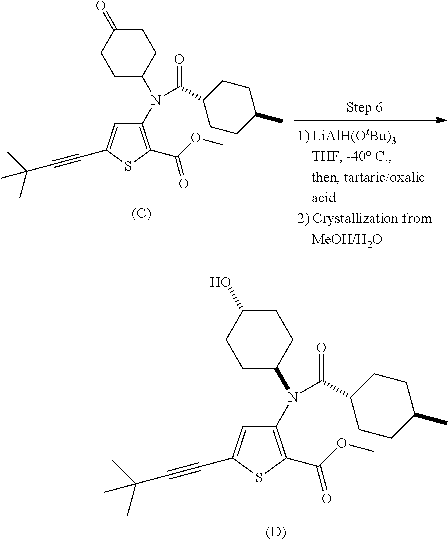

Compound (C) (399 g, 1.0 eq, limiting reagent) was charged to a 12 L reactor and purged with N₂. Anhydrous THF (2 L, 5.0 vol) was then charged to the reactor, then the mixture was agitated. The resulting solution was cooled to −65 to −64° C.

LiAlH(OtBu)₃ (960 ml of 1 M in THF, 2.40 vol or 1.1 eq) was added while maintaining not higher than −40° C. batch temperature. The solution was added over 2 hours and 15 minutes. The rate of addition was 1.45 vol/h.

Upon completion of LiAlH(OtBu)₃ addition, the batch was stirred at −40° C. or lower temperature for 1 additional hour. A small IPC sample was collected after 1 h and immediately quenched with 1 N HCl. The sample was analyzed for Compound (C) consumption (the reaction was judged complete when Compound (C) was ≦0.5% with respect to Compound (D) by IPC method).

If reaction was not completed, stir reaction at −40° C. for an additional hour. An IPC sample was collected and immediately quenched with 1 N HCl. If reaction was not completed, then additional amount of LiAlH(OtBu)₃ was added (for instance, if 1.0% peak area of unreacted Compound (C) remained compared to product Compound (D), then 2% of the original charge of LiAlH(OtBu)₃ solution was added). The batch was kept at −40 to −50° C. or lower temperature during reaction. Upon addition of LiAlH(OtBu)₃, the batch was stirred for 1 hour at −45 to −40° C. A small IPC sample was collected and immediately quenched with 1 N HCl.

Once the reaction was complete, MTBE (1197 L, 3 vol) was charged to the batch, then the batch was warmed to 0° C. The resulting solution was added over about 10-15 minutes to a mixture of aqueous oxalic acid (or tartaric acid) which was prepared by cooling a mixture of oxalic acid (or tartaric acid) (9% w/w, 2394 L, 6 vol) and MTBE (7 L, 2 vol) to 8-10° C. The batch temperature was adjusted to 15-25° C. and the resulting mixture was stirred for 30-60 minutes.

The agitation was stopped. The upper organic phase was collected. Water (2.8 L, 7 vol) was added to the organic phase. The biphasic mixture was stirred for 10 minutes at 15-25° C. Then agitation was stopped. The upper organic phase was collected.

Crystallization of Compound (D) was performed by switching solvent to methanol. The batch volume was reduced to 1.2 L or 3.0 vol by vacuum distillation at <60° C.

Methanol (4 L, 10 vol) was added to the batch (without adjusting batch temperature) and the batch volume was reduced to 1.2 L or 3.0 vol by vacuum distillation at <60° C. This step was repeated. Then, the batch volume was adjusted to 3.0 vol by addition of 479 mL.

A small IPC sample of the slurry was collected. The solids were filtered and the solution was analyzed by gas chromatography to determine the level of residual THF and MTBE with respect to methanol. If solvent switch to methanol was complete, then the batch was heated to 60-65° C. and stirred at this temperature until all solids dissolved. 2 volumes of the 50 vol % methanol/water solution was added, maintaining the temperature at not less than (NLT) 50° C. Then, the temperature was adjusted to 47-53° C. (e.g., 50° C.), and the temperature was maintained for 4 hours in order for solids to start crystallizing. Then, the remaining 2 volumes of the 50 vol % methanol/water solution was added into the batch. The batch was then cooled 15-25° C. at approximately 5° C./hour, and was held for not less than (NLT) 4 hours at 15-25° C. The filter cake was washed with 1 volume (based on compound 5 charge) of 50 volume % methanol/water

The material was dried for at least 12 hours under vacuum with nitrogen bleed at 55-65° C.

If required, the batch could be recrystallized by charging dry Compound (D) (1 equiv) and methanol (2 vol, relative to Compound (D) charge) to a reactor and heating the batch to 60-65° C. until all solids dissolved. The batch would then be cooled to −20° C. over a 3 hour period. The resulting solids would be filtered and dried for at least 12 hours under vacuum with nitrogen bleed at 55-65° C. Compound D: ¹H NMR (400 MHz, DMSO-d₆) δ 0.52-0.69 (m, 2H), 0.75 (d, 6.4 Hz, 3H), 0.76-0.86 (m, 1H), 1.11-1.24 (m, 5H), 1.31 (s, 9H), 1.43-1.57 (m, 6H), 1.73-1.83 (m, 4H), 3.17-3.18 (m, 1H), 3.75 (s, 3H), 4.24-4.30 (m, 1H), 4.49 (d, J=4.4 Hz, 1H), 7.23 (s, 1H).

B. Method B: Reducing Reagents Other than LiAlH(OtBu)₃

Reducing reagents other than LiAlH(OtBu)₃ that gave predominantly the desired isomer were: LiAlH(OiBu)₂(OtBu)₃, DiBAlH, LiBH4, NaBH4, NaBH(OAc)₃, Bu₄NBH₄, ADH005 MeOH/KRED recycle mix A, KRED-130 MeOH/KRED recycle mix A, Al(Oi-Pr)₃/i-PrOH, and (i-Bu)₂AlOiPr.

### 7. Step 7

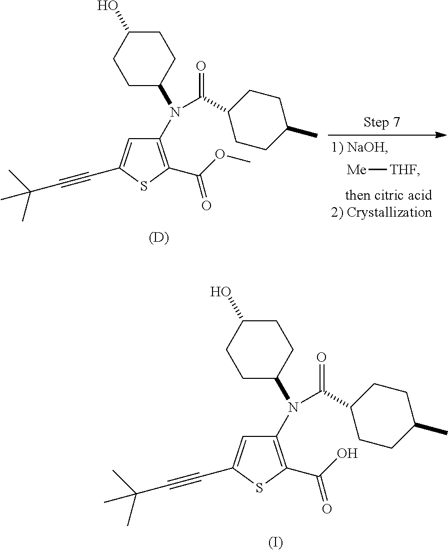

Compound (D) and Me-THF (5 volumes, based on compound 6 charge) were added to a reactor. To the solution, an aqueous solution of NaOH (2N, 4.0 vol, 3.7 equiv) was added at 15-25° C. The batch was heated to 68-72° C. and stirred for 8-16 hours at this temperature. The reaction progress was monitored by LC. Upon completion, the batch was cooled to 0-5° C. Precipitates formed. An aqueous solution of citric acid (30% by weight, 3.7 equiv), was added over 15-30 minutes, while maintaining the batch temperature below 25° C. The phases were separated. Water was added (5 volumes based on compound 6 charge) to the organic layer. The phases were separated. The batch volume was reduced to 3 volumes (based on compound (D) charge) via vacuum distillation at a maximum temperature of 35° C. Then dry Me-THF (3 vol, based on compound (D) charge) was added. The water content was determined by Karl Fisher titration. The batch is deemed dry if residual water level is ≦1.0%.

Optionally, the final product of Compound (1) can be recrystallized either in EtOAc or in a mixture of nBuOAc and acetone via solvent switch described below to form Form M of Compound (1):

A: Recrystallization in a Mixture of nBuOAc and Acetone:

A solvent switch from 2-Me-THF to nBuOAc was performed by first reducing the batch volume to 2-3 volumes (based on compound (D) charge) by vacuum distillation at a maximum temperature of 45° C. nBuOAc (3 vol, based on compound (D) charge) was added and the batch volume was reduced to 2-3 volumes (based on compound (D) charge) via vacuum distillation at a maximum temperature of 45° C. The batch volume was then adjusted to a total of 5-6 volumes by addition of nBuOAc. The solution was analyzed for residual 2-Me-THF in content in nBuOAc. This cycle was repeated until less than 1% of 2-Me-THF with respect to nBuOAc remained, as determined by GC analysis. Once the residual 2-Me-THF IPC criterion was met and it was insured that the total batch volume is 6 (based on compound (D) charge), the batch temperature was adjusted to 40-45° C. Acetone is then charged into the batch to have approximately 10 wt % acetone in the solvent. The batch temperature was aduhsted to 40-45° C. Compound 1 seed (1.0% by weight with respect to the total target weight of compound (1)) was added. The batch was agitated at 40-45° C. for 4-8 hours. The rcrystallization progress is monitored by X-ray powder diffraction (XRPD). If spectrogram matched that of required form, then the batch was cooled from 40-45° C. to 30-35° C. (preferably about 35° C.) at rate of 5° C./hour. The batch was held at about 35° C. for at least one hour, and then filtered and the filter cake was washed with 9:1 wt:wt mixture of nBuOAc/acetone (1 vol). The material was dried in vacuum with nitrogen bleed at NMT 45° C. for 12-24 hours. The expected isolated molar yield of compound (1) (Form M) starting with compound (D) was 80-85%. Compound (1): ¹H NMR (400 MHz, DMSO-d₆) δ0.58 (m, 1H), 0.74 (q, J=6.53 Hz, 1H), 0.81 (ddd, J=12.86, 12.49, 3.19 Hz, 1H), 1.18 (m, 5H), 1.28 (s, 3H), 1.42 (m, 1H), 1.55 (m, 3H), 1.61 (m, 1H), 1.73 (m, 2H), 1.81 (m, 2H), 3.19 (m, 1H), 4.26 (m, 1H), 4.49 (bs, 1H), 7.14 (s, 1H), 13.45 (bs, 1H).

### B: Recrystallization in EtOAc:

A solvent switch from 2-Me-THF to EtOAc was performed by first reducing the batch volume to 2-3 volumes (based on compound (D) charge) by vacuum distillation at a maximum temperature of 35° C. EtOAc (10 vol, based on compound (D) charge) was added and the batch volume was reduced to 2-3 volumes (based on compound (D) charge) via vacuum distillation at a maximum temperature of 35° C. The solution was analyzed for residual 2-Me-THF in content in EtOAc. This cycle was repeated until less than 1% of Me-THF with respect to EtOAc remained, as determined by GC analysis. Once the residual 2-Me-THF IPC criterion was met and it was insured that the total batch volume is 10 (based on compound (D) charge), the batch temperature was adjusted to 40-45° C. Compound 1 seed (1.0% by weight with respect to the total target weight of compound (1)) was added. The batch was agitated at 40-45° C. for 12 hours. A flat floor/flat bottomed reactor (not conical) should be used. The rcrystallization progress is monitored by X-ray powder diffraction (XRPD). If spectrogram matched that of required form, then the batch was cooled from 40-45° C. to 11-14° C. at rate of 5° C./hour. The batch was filtered and the filter cake was washed with EtOAc (1 vol), previously chilled to 11-14° C. The material was dried in vacuum with nitrogen bleed at NMT 45° C. for 12-24 hours. The expected isolated molar yield of compound (1) (Form M) starting with compound (D) was 80-85%.

### Example 3

### Formation of Polymorphic Form A and Polymorphic Form M of Compound (1)

3A: Formation of Polymorphic Form A of Compound (1)

Polymorphic Form A of Compound (1) can be prepared by following the steps described below:

10 g of Compound (1) was charged to a reactor. 20 g of methanol was then charged to the reactor. The reactor was heated to 60° C. to dissolve Compound (1). The reactor was then cooled to 10° C., and left until solids of Compound (1) formed. The solids of Compound (1) were filtered. 20 g of acetone at 25° C. was added to the solids of Compound (1). The mixture of acetone and Compound (1) was stirred for 1 hour and the resulting solids were filtered. The filtered solids were dried at 75° C. for 12 hours.

Characteristics of Form A of Compound (1): XRPD data and C¹³ solid state NMR data of Form

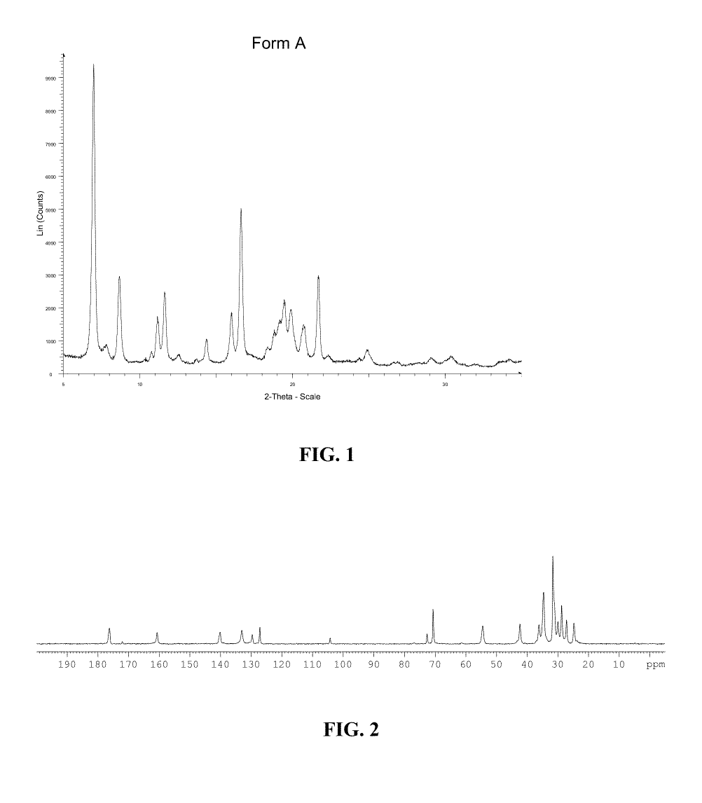

A of Compound (1) are shown in FIG. 1 and FIG. 2, respectively. Certain representative XRPD peaks and DSC endotherm (° C.) of Form A of Compound (1) are summarized in Table 1 below.

TABLE 1
Certain representative XRPD Peaks and DSC Endotherm of Form A
Form A
DSC Endotherm (° C.) 188° C.

| XRPD Peaks | Angle (2-Theta ± 0.2) | Intensity % |
|---|---|---|
| 1 | 6.9 | 100.0 |
| 2 | 16.6 | 53.3 |
| 3 | 21.7 | 31.6 |
| 4 | 8.6 | 31.3 |
| 5 | 11.6 | 26.2 |
| 6 | 19.4 | 23.8 |

3B: Formation of Polymorphic Form M of Compound (1)

### 1. Method A

Polymorphic Form M of Compound (1) can be prepared by following the steps described below:

10 g of Compound (1) was charged to a reactor. 50 g of ethyl acetate was then charged to the reactor. The reactor was heated to 45° C. and the mixture was stirred for 1-2 days until Form M was observed. Then, the reactor was cooled to 25° C., and left until solids of Compound (1) formed. The solids of Compound (1) were filtered and the filtered solids were dried at 35° C. for 24 hours.

### 2. Method B

Polymorphic Form M of Compound (1) was also be prepared in a similar manner as described above for Method A but employing a solvent system listed in Table 2A below and stirring Compound (1) in the solvent system at a respective temperature range listed in Table 2A.

TABLE 2A
Conditions for the Preparation of Form M

| Solvents | Form M Temperature Window |
|---|---|
| n-BuOAc | 35-47° C. |
| n-BuOAc/Acetone (90%/10%, w/w) | 30-47° C. |
| n-BuOAc/MeOAc (50%/50%, w/w) | 25-47° C. |
| Acetone | 20-47° C. |
| MEK | 30-47° C. |
| n-BuOAc/Heptane (50%/50%, w/w) | 25-47° C. |
| Acetone/Heptane (50%/50%, w/w) | 25-47° C. |
| EtOAc/Heptane (50%/50%, w/w) | 25-47° C. |
| EtOAc | 45-47° C. |

Characteristics of Form M of Compound (1): XRPD data and C¹³ solid state NMR data of Form M of Compound (1) are shown in FIG. 3 and FIG. 4, respectively. Certain representative XRPD peaks and DSC endotherm (° C.) of Form M of Compound (1) are summarized in Table 2B below.

TABLE 2B
Certain representative XRPD Peaks and DSC Endotherm of Form M
Form M
DSC Endotherm (° C.) 230° C.

| XRPD Peaks | Angle (2-Theta ± 0.2) | Intensity % |
|---|---|---|
| 1 | 19.6 | 100.0 |
| 2 | 16.6 | 72.4 |
| 3 | 18.1 | 59.8 |
| 4 | 9.0 | 47.6 |
| 5 | 22.2 | 39.9 |
| 6 | 11.4 | 36.6 |

### Example 4

### Formation of Tromethamine Salt of Compound (1)

10 g of Compound (1) was dissolved in 100 g of IPA (isopropyl alcohol). A stock solution of 1 equivalent of tromethamine in water (2.72 g of tromethamine and 6.34 g of water) was prepared. 0.5 g of the tromethamine stock solution was added into the solution of Compound (1). The mixture was hold for 1 hour optionally with a seed of tromethamine salt of Compound (1) therein. The resulting slurry was filtered and washed with IPA. The filtered wet cake was dried at 70° C. Yield 58%.

### Example 5

### Preparation of Capsules Comprising Polymorphic Form A of Compound (1)

Two different oral dosage formulations of Form A of Compound (1) were prepared as shown in Tables 3a and 3b.

TABLE 3a
200 mg Form A Capsule formulation

| Ingredients | Amounts (mg) | Percent |
|---|---|---|
| Form A of | 200.00 | 52.00 |
| Compound (1) | | |
| Avicel PH 101 | 42.3 | 11.00 |
| Lactose | 53.8 | 14.00 |
| Monohydrate | | |
| Poloxamer 188 | 13.5 | 3.50 |
| Sodium Lauryl | 7.7 | 2.00 |
| Sulfate | | |
| Povidone K29/32 | 19.2 | 5.00 |
| Avicel PH 102 | 11.5 | 3.00 |
| Lactose | 11.5 | 3.00 |
| Monohydrate | | |
| Crosscarmellose | 21.2 | 5.50 |
| Sodium | | |
| Magnesium Stearate | 3.8 | 1.00 |
| Total Formulation | 384.62 | 100.00 |
| Weight (mg) | | |

Final Weight

| | |
|---|---|
| Hard gelatin | 100 |
| Capsule white | |
| opaque, size 0 | |
| Total Weight | 484.62 |

TABLE 3b
50 mg Form A Capsule formulation

| Ingredients | Amounts (mg) | Percent |
|---|---|---|
| Form A of | 50.00 | 11.00 |
| Compound (1) | | |
| Avicel PH 101 | 63.64 | 14.00 |
| Lactose | 172.73 | 38.00 |
| Monohydrate | | |
| Poloxamer 188 | 15.91 | 3.50 |
| Sodium Lauryl | 9.09 | 2.00 |
| Sulfate | | |
| Povidone K29/32 | 22.73 | 5.00 |
| Avicel PH 102 | 36.36 | 8.00 |
| Lactose | 54.55 | 12.00 |
| Monohydrate | | |
| Crosscarmellose | 25.00 | 5.50 |
| Sodium | | |
| Magnesium Stearate | 4.55 | 1.00 |
| Total Formulation | 454.55 | 100.00 |
| Weight (mg) | | |

Final Weight

| | |
|---|---|
| Hard gelatin | 100 |
| Capsule white | |
| opaque, size 0 | |
| Total Weight | 554.55 |

A. Wet granulation and Capsule Composition

200 mg Form A capsules were prepared as follows. 50 mg Form A capsules were prepared in a similar manner as described below for 200 mg capsules. The formulation compositions for both the wet granulation and capsules blends of the active capsule are described in Tables 4a and 4b.

TABLE 4a
Polymorphic Form A of Compound (1) (200
mg) Wet granulation Composition

| | Amount | |
|---|---|---|
| | (mg) per | |
| Component | capsule | % W/W |
| Compound (1) crystalline (Form A) | 200.00 | 59.43 |
| Avicel PH-101 (microcrystalline | 42.31 | 12.57 |
| cellulose), NF, PhEur, JP | | |
| Lactose Monohydrate 80, NF, PhEur, JP | 53.85 | 16.00 |
| Poloxamer 188 NF, PhEur, JP | 13.46 | 4.00 |
| Sodium Lauryl Sulfate NF, PhEur, JP | 7.69 | 2.29 |
| Povidone K29/32 USP | 19.23 | 5.71 |
| Total | 336.54 | 100.00 |

TABLE 4b
Polymorphic Form A of Compound (1)
(200 mg) Capsule Composition

| | Amount | |
|---|---|---|
| | (mg) per | |
| Component | capsule | % W/W |
| Compound (1) Granulation (Milled) | 336.54 | 87.50 |
| Avicel PH-102 (microcrystalline | 11.54 | 3.00 |
| cellulose), NF, PhEur, JP | | |
| Lactose Monohydrate 80, NF, PhEur, JP | 11.54 | 3.00 |
| Ac-Di-Sol (cross carmellose sodium), NF, PhEur, JP | 21.15 | 5.50 |
| Magnesium Stearate NF, PhEur, JP | 3.85 | 1.00 |
| Total | 384.62 | 100.00 |

The actual weights of each ingredient for the final capsule blend of the 200 mg capsule strength batch can be determined based on the yield calculations of the wet granulation (internal Phase). Sample calculation below:

$$Weight of Excipient = \frac{\begin{matrix}Wet Granulation yield \% \times \\ Theorectical Weight of Excipient ( kg )\end{matrix}}{100}$$

### B. Wet Granulation and Capsule Preparation Overview (200 mg)

### a) High Shear Wet Granulation Process Flow

1. An excess (10%) amount of polymorphic Form A of Compound (1), Avicel PH-101, Lactose Monohydrate, Poloxamer 188, Sodium Lauryl Sulfate, and Povidone K29/32 were weighed.
2. Using the Co-mill equipped with a #20 mesh screen, the excess amount of Compound (1), Avicel PH-101, Lactose Monohydrate, Poloxamer 188, Sodium Lauryl Sulfate, and Povidone K29/32 were screened at 70% speed.
3. The required amount of “sieved” Compound (1), Avicel PH-101, Lactose Monohydrate, Poloxamer 188, Sodium Lauryl Sulfate, and Povidone K29/32 were weighed and transferred to a V-Shell blender (PK 1 cu.ft.).
4. The materials were blended for 5 mins at the set speed (typically 25 RPM).
5. The bulk wet granulation blend was placed in a High shear granulator (Vector GMX.01).
6. The blend was granulated.
7. Once the granulation end point is achieved, the material (Wet granulation blend) was transferred into a suitable container and dried.
8. Using the Co-mill with #20 mesh screen, all the dry granulations was milled.

### b) Capsule Manufacturing Process Flow

9. An excess (10%) amount of Avicel PH-102, Lactose Monohydrate, Crosscarmellose Sodium, and Magnesium Stearate were weighed.
10. Using the Co-mill equipped with a #20 mesh screen, the excess amounts of Avicel PH-102, Lactose Monohydrate, Crosscarmellose Sodium, and Magnesium Stearate were screned at 70% speed.
11. The required amount of “sieved” Avicel PH-102, Lactose Monohydrate, Crosscarmellose Sodium, Magnesium Stearate, and milled granulation were weighed and transferred to a V-Shell blender (PK 1 cu.ft.), except the magnesium stearate.
12. The materials in the V-Shell blender were blended.
13. Magnesium stearate was then added into the V-shell blender, and the mixture was blended.
14. Encapsulate the final blend.

### Example 6

### Preparation of Tablets Comprising Polymorphic Form M of Compound (1)

### A. Tablets A

Wet Granulation and Tablet Composition

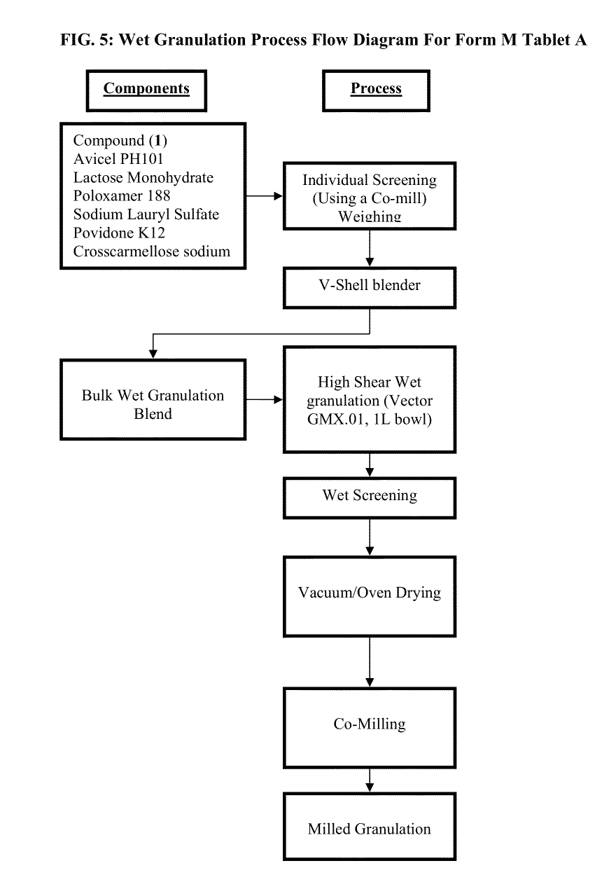

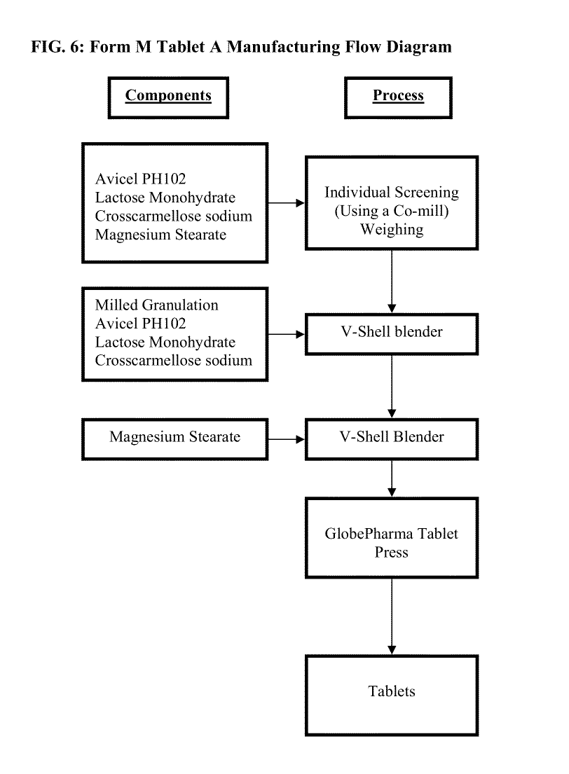

The formulation compositions for both the wet granulation and tablet blends of the active tablets are described in Tables 5a and 5b. The overall composition specification of the tablets is described in Table 5c. A flow diagram for a wet granulation process and a manufacturing flow diagram for Form M Tablet A are shown in FIGS. 5 and 6, respectively.

TABLE 5a
Form M (250 mg) Wet granulation Composition

| | Amount | |
|---|---|---|
| | (mg) per | |
| Component | tablet | % W/W |
| Compound (1) crystalline (Form M) | 250.00 | 57.46 |
| Avicel PH-101 (microcrystalline | 52.88 | 12.15 |
| cellulose), NF, PhEur, JP | | |
| Lactose Monohydrate, #316, NF, PhEur, JP | 67.31 | 15.47 |
| Poloxamer 188 NF, PhEur, JP | 16.83 | 3.87 |
| Sodium Lauryl Sulfate NF, PhEur, JP | 9.62 | 2.21 |
| Povidone K12 USP | 24.04 | 5.53 |
| Ac-Di-Sol (cross carmellose sodium), NF, PhEur, JP | 14.42 | 3.31 |
| Total | 435.10 | 100.00 |

TABLE 5b
Form M (250 mg) Tablet Composition

| | Amount | |
|---|---|---|
| | (mg) per | |
| Component | tablet | % W/W |
| Compound (1) Granulation (Milled) | 435.10 | 78.50 |
| Avicel PH-102 (microcrystalline | 83.14 | 15.00 |
| cellulose), NF, PhEur, JP | | |
| Lactose Monohydrate, #316, NF, PhEur, JP | 16.63 | 3.00 |
| Ac-Di-Sol (cross carmellose sodium), NF, PhEur, JP | 13.86 | 2.50 |
| Magnesium Stearate NF, PhEur, JP | 5.54 | 1.00 |
| Total | 554.27 | 100.00 |

TABLE 5c
Form M (250 mg) Tablet Overall Composition

| | | | % in |
|---|---|---|---|
| | | % in dry | core |
| | | granule | tablet |
| intra- | Form M of Compound (1) | 57.46 | 45.10 |
| granular | Avicel PH-101, NF, PhEur, JP | 12.15 | 9.54 |
| | Lactose Monohydrate, #316, NF, PhEur, | 15.47 | 12.14 |
| | JP | | |
| | Ac-Di-Sol, NF, PhEur, JP | 3.31 | 2.60 |
| | Sodium Lauryl Sulfate, NF, PhEur, JP | 2.21 | 1.74 |
| | Poloxamer 188, NF, PhEur, JP | 3.87 | 3.04 |
| | Povidone K12, USP | 5.53 | 4.34 |
| | Water, USP | na | na |
| | total granules: | 100.00 | 78.50 |
| extra- | Avicel PH-101, NF, PhEur, JP | | 15.00 |
| granular | Lactose Monohydrate, #316, NF, PhEur, | | 3.00 |
| | JP | | |
| | Ac-Di-Sol, NF, PhEur, JP | | 2.50 |
| | Magnesium Stearate, NF, PhEur, JP | | 1.00 |
| | total core tablet: | | 100.00 |

### a) High Shear Wet Granulation Process Flow

1. An excess (10%) amount of Compound (1), Avicel PH-101, Lactose Monohydrate, Poloxamer 188, Sodium Lauryl Sulfate, Povidone K12, and Cross Carmellose Sodium were weighed.
2. Using the Co-mill equipped with an 813 μm mesh screen, the excess amount of Compound (1), Avicel PH-101, Lactose Monohydrate, Poloxamer 188, Sodium Lauryl Sulfate, Povidone K12, and Cross Carmellose Sodium were screened at 30% speed. The sieved materials were placed in individual bags or containers.
3. The required amount of “sieved” Compound (1), Avicel PH-101, Lactose Monohydrate, Poloxamer 188, Sodium Lauryl Sulfate, Povidone K12, and Cross Carmellose Sodium were weighed.
4. A V-Shell blender was set up and the materials from step 3 were transferred into a blender.
5. The materials were blended in the V-Shell blender for 5 mins at the set speed (typically 25 RPM).
6. The contents of the V-Shell blender were emptied into LDPE bags (Bulk Wet Granulation blend).
7. A High shear granulator (Vector GMX.01) with a 1 L granulator bowl was set up.
8. The bulk wet granulation blend was then transferred into the 1 L granulator bowl.
9. The blend was granulated according to the prescribed wet granulation parameters (Table 6)
   Stage 1: 77% of the total amount of water required for the wet granulation was used to granulate the material at the prescribed process parameters. Once the water addition was complete, the granulation was stopped. The walls, impeller, and chopper of the high shear granulator was scraped and the granulation was verified to determine if the visual endpoint was reached. If YES moved on to step 10, if NO proceeded to stage 2
   Stage 2: the remaining 23% of water was added and the material was granulated at the prescribed process parameters. Once the water addition was completed, the granulation was stopped and the walls, impeller, and chopper of the high shear granulator were scraped and the granulation was verified to determine if the visual endpoint was reached. If YES moved on to step 10, if NO continued to granulate at the preceding process parameters with 2 ml portions of water until the end-point was reached.
10. Once the granulation end point was achieved, the material (Wet granulation blend) was screened through a #20 (850 μm) mesh screen and the screened material was transferred into a suitable container.
11. The screened material from step 10 was dried in an oven according to the prescribed drying parameters (overall drying temperature: 30° C.-45° C.).
12. Using the Co-mill with an 813 μm mesh screen, all the dry granulations were milled at 30% speed. (Hand screen any material left over in the Co-mill through a #20 (850 μm) mesh screen, and combine both the milled and screened granulations). The weight of the milled granulation was determined and the material was packaged in bags.

### b) Tablet Manufacturing Process Flow

1. An excess (10%) amount of Avicel PH-102, Lactose Monohydrate, Crosscarmellose Sodium, and Magnesium Stearate were weighed.
2. Using the Co-mill equipped with an 813 μm mesh screen, the excess amounts of Avicel PH-102, Lactose Monohydrate, Crosscarmellose Sodium, and Magnesium Stearate were screened at 30% speed.
3. The required amount of “sieved” Avicel PH-102, Lactose Monohydrate, Crosscarmellose Sodium, Magnesium Stearate, and milled granulation were weighed.
4. The materials was transferred into a V-Shell blender, except the magnesium stearate.
5. The materials in the V-Shell blender was blended for 10 mins at the set speed (typically 25 RPM).
6. The magnesium stearate was then into the V-shell blender.
7. The materials in the V-Shell blender were blended for 1 min at the set speed (typically 25 RPM).
8. The contents of the V-Shell blender was emptied into a bag.
9. A GlobePharma tablet press with the modified caplet tooling (size 0.30″×0.60″) was set up.
10. The final blend was compressed to form tablets.

TABLE 6
Wet granulation process variables, settings and targets

| | | Target Initial |
|---|---|---|
| | Overall | Conditions (or |
| Variable | Setting/Range | Center points) |
| Co-mill speed | 10-80% | 30% |
| (% total speed) | | |
| For 250 mg tablet | 15%-25% of | 18% of |
| Amount of water (ml) | granulation | granulation |
| | blend amount | blend amount |
| Rate of water | N/A | 2.0 ml/min |
| addition (ml/min) | | |

### B. Tablets B

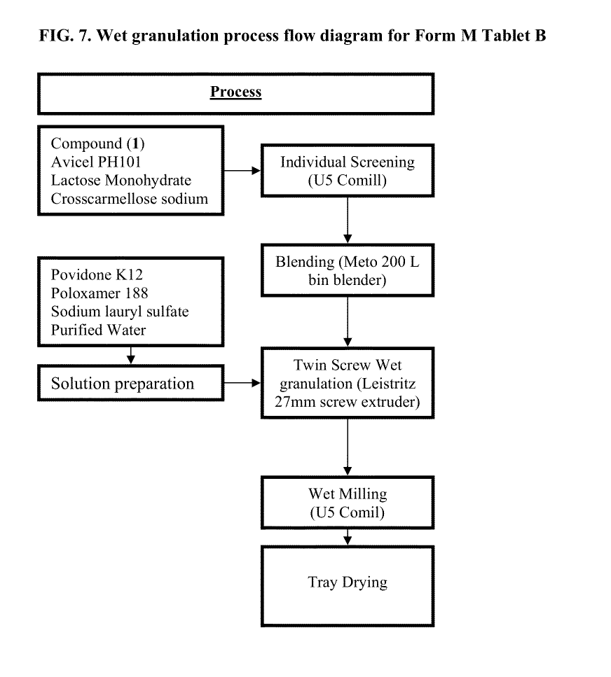

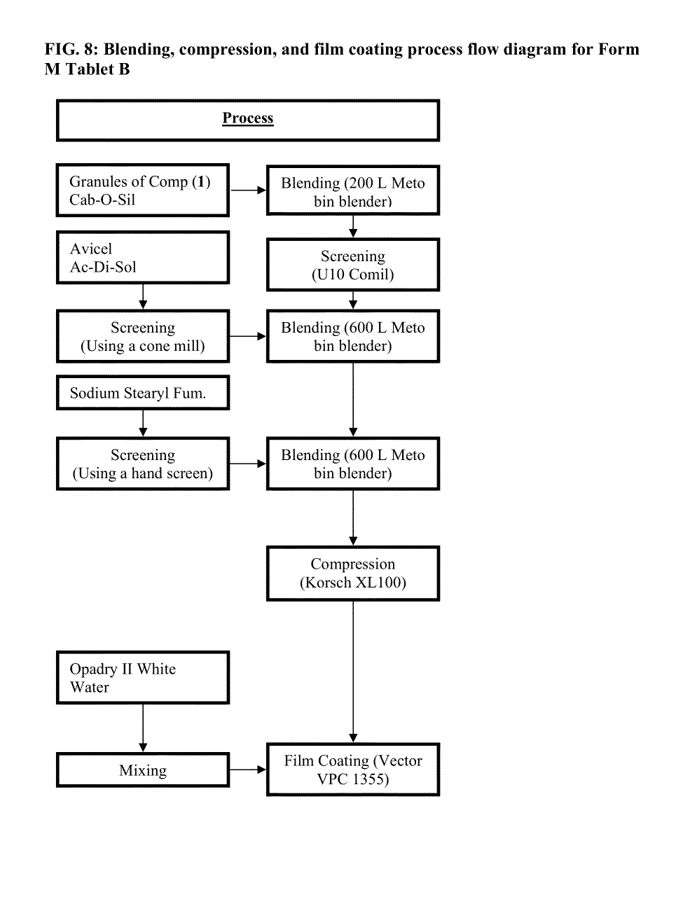

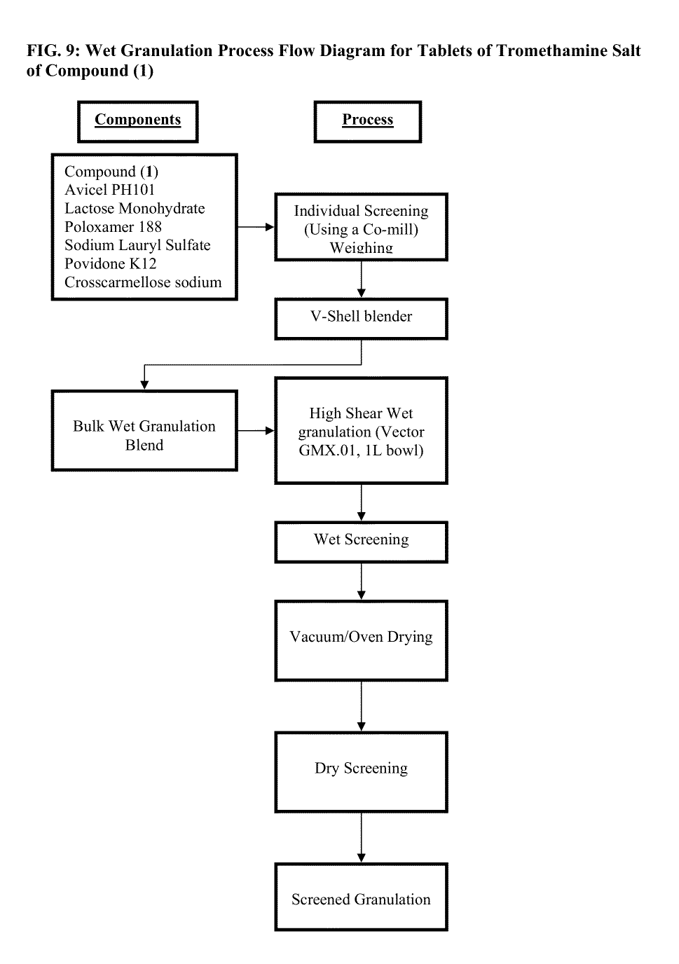

The formulation composition for the pre granulation blend is given in Table 7a. Table 7b gives the composition of the granulation binder solution. The theoretical compression blend composition is given in STable 7c. The composition and approximate batch size of the film coating suspension (including 50% overage for line priming and pump calibration) is given in Table 7d. The overall specification of the tablets B composition is summarized in Table 7e. The target amount of the film coating is 3.0% w/w of the core tablet weight. A flow diagram for a wet granulation process and a manufacturing flow diagram for Form M Tablet B are shown in FIGS. 7 and 8, respectively.

TABLE 7a
Pre-granulation composition

| Component | % W/W |
|---|---|
| Compound (1) crystalline (Form M) | 64.81 |
| Avicel PH-101 (microcrystalline cellulose), NF, PhEur, JP | 13.67 |
| Lactose Monohydrate, #316, NF, PhEur, JP | 17.76 |
| Ac-Di-Sol (cross carmellose sodium), NF, PhEur, JP | 3.76 |
| Total | 100.00 |

TABLE 7b
Binder solution composition

| Component | % W/W |
|---|---|
| Sodium Lauryl Sulfate, NF, PhEur, JP | 11.75 |
| Poloxamer 188, NF, PhEur, JP | 20.80 |
| Povidone K12, USP | 20.80 |
| Water | 46.65 |
| Total | 100.00 |

TABLE 7c
Compression blend composition

| Component | % W/W |
|---|---|
| Compound (1) TSWG granulation | 60.20 |
| Avicel PH-101, NF, PhEur, JP | 34.86 |
| Ac-Di-Sol, NF, PhEur, JP | 1.93 |
| Cab-O-Sil M5P, NF, PhEur, JP | 0.60 |
| Sodium Stearyl Fumarate, NF, PhEur, JP | 2.42 |
| Total | 100.00 |

TABLE 7d
Film coat suspension composition

| Component | % W/W |
|---|---|
| Opadry II White, 85F18378 | 20.00 |
| Water, USP | 80.00 |
| Total | 100.00 |

TABLE 7e
Overall Composition of Tablets B

| | | % in pre- | % in | % in | % in |
|---|---|---|---|---|---|
| | | granulation | dry | core | coated |
| | | blend | granule | tablet | tablet |
| intra- | Compound (1) (Form M) | 64.81 | 58.14 | 35.00 | 33.98 |
| granular | Avicel PH-101, NF, PhEur, JP | 13.67 | 12.27 | 7.39 | 7.17 |
| | Lactose Monohydrate, #316, NF, PhEur, JP | 17.76 | 15.93 | 9.59 | 9.31 |
| | Ac-Di-Sol, NF, PhEur, JP | 3.76 | 3.37 | 2.03 | 1.97 |
| | total pre-granulation blend: | 100.00 | 89.71 | 54.01 | 52.43 |
| in binder | Sodium Lauryl Sulfate, NF, PhEur, JP | | 2.27 | 1.37 | 1.33 |
| solution | Poloxamer 188, NF, PhEur, JP | | 4.01 | 2.42 | 2.34 |
| | Povidone K12, USP | | 4.01 | 2.42 | 2.34 |
| | Water, USP | | na | na | na |
| | total granules: | | 100.00 | 60.20 | 58.45 |
| extra- | Avicel PH-101, NF, PhEur, JP | | | 34.86 | 33.85 |
| granular | Ac-Di-Sol, NF, PhEur, JP | | | 1.93 | 1.87 |
| | Cab-O-Sil M5P, NF, PhEur, JP | | | 0.60 | 0.58 |
| | Sodium Stearyl Fumarate, NF, PhEur, JP | | | 2.42 | 2.34 |
| | total core tablet: | | | 100.00 | 97.09 |
| coating | Opadry II White, 85F18378 | | | | 2.91 |
| | Water, USP | | | | na |
| | total final tablet: | | | | 100.00 |

### A. Wet Granulation

a) Binder Solution Preparation

The binder solution included the Povidone, SLS, and Poloxamer. The solution was prepared based on 9% w/w water content of the final dry granulation. An excess amount of 100% were prepared for pump calibration, priming lines, etc.
1. The required amount of Poloxamer 188, Sodium Lauryl Sulfate, Povidone K12, and purified (DI) water were weighed.
2. Under constant stirring, was add the Povidone K12 to the DI water, and the resulting mixture was stirred. Poloxamer 188 and Sodium Lauryl Sulfate were added into the tank containing the DI water and dissolved Povidone K12. The stir rate was then turned down after the surfactant addition such that only a partial vortex formed.
3. The solution was stirred until all the solids present were visually fully dissolved.
4. The solution was then sit at least 2 hours until air bubbles in solution disappeared. Alternatively, a partial vacuum could be pulled on the solution tank for up to an hour to degas the solution.

b) Wet Granulation Process
1. Compound (1), Croscarmellose Sodium, Avicel PH-101, and Lactose Monohydrate were weighed.
2. Using a US or U10 Comill equipped with a 32R screen and round impeller, the weighed out Compound (1), lactose, and avicel were delumped respectively at 4000 rpm in the U5, or 2800 rpm in the U10 into bags or directly into the Meto 200 L blender.
3. The materials were transferred from step 2 into a Meto 200 L bin blender.
4. The materials were blended for 25 minutes at 10 RPM.
5. The materials were charged into a loss in weight powder feeder directly from the blend shell, or into a LDPE bag.
6. A Leistritz 27 mm twin screw extruder with the required barrel and screw configuration specified in Tables 8a and 8b were set up.
7. The dry blend was fed into the extruder using a K-Tron loss in weight feeder.
8. The binder fluid was injected into the extruder using a calibrated K-Tron liquid pump. The pump was calibrated using the actual fluid prior to operation.
9. The blend was then granulated.
10. The weight ratio of solution feed rate over powder feed rate was 0.215 to have the proper final composition. For the intended powder feed of 167.00 g min⁻¹, the solution feed rate was 35.91 g min⁻¹.
11. The wet granules coming out of the twin screw was milled using an inline U5 Comil at 1000 rpm with square 4 mm screen and round bar impeller.
12. The wet milled granules were collected and dried. The water content was NMT 3.0%.

TABLE 8a
27-mm Leistritz Twin Screw Extruder barrel configuration

| Barrel Number | Barrel Configuration |
|---|---|
| 1 | Blank, or Plugged Vent |
| 2 | Blank, or Plugged Vent |
| 3 | Blank, or Plugged Vent |
| 4 | Blank, or Plugged Vent |
| 5 | Blank, or Plugged Vent |
| 6 | Feed |
| 7 | Liquid injection (nozzle orifice is 0.7 mm) |
| 8 | Blank, or Plugged Vent |
| die config | no die |

TABLE 8b
27-mm Leistritz Twin Screw Extruder screw configuration
Screw configuration (tail to tip)

Spacers for rest of screw shaft
GFA-2-30-90
GFA-2-30-90
GFA-2-30-30
GFA-2-20-90
2-row, 5-tooth per row combing element
GFA-2-30-60
Tip

### B. Extra-Granular Blending and Compression Process

1. The quantity of the extra-granular excipients based on the compression blend composition were weighed.
2. The granules and Cab-O-Sil was added directly to the 200 L Meto bin blender and blended for 8 minutes at 15 RPM.
3. The blend was then passed through a U10 Comil with a 40G screen and round bar impeller at 600 rpm directly into the 600 L Meto bin blender or into double LDPE bags.
4. Approximate amounts of Avicel PH-101 and Ac-Di-Sol were screened using a U10 Comil with a 32R screen and round bar impeller at 600 rpm directly into the 600 L Meto bin blender or into double LDPE bags.
5. Sodium stearyl (SSF) was hand screened through a #50 mesh screen into an appropriate container. A portion of the extra granular blend equal to roughly 10 times by mass the amount of SSF calculated in step one was placed in the container with the SSF and blended for 30 seconds before the mixture was added to the bin blender.
6. The mixture was blended for 10 minutes at 15 rpm.
7. The final blend was compressed.
8. During the compression process, the individual and average tablet weights, hardness, and thickness was measured.

### C. Film Coating Process

A film coating was applied to the core tablets in a Vector VPC 1355 pan coater as a 20 wt % Opadry II white #85F18378 aqueous suspension. The target coating was 3.0% w/w of the core tablet weight, with an acceptable range of 2.5% to 3.5%. To accomplish this, an amount of coating suspension equivalent to a 3.2% weight gain was sprayed, which would give a 3.0% coating assuming a coating efficiency of 95%. The film coating process was performed as follows:
1. Calculate the pan load by dividing the tablet yield by 3 (or 2 if there are less than 75 kg of core tablets) and calculate the required amount of coating suspension (based on 3.2% coating), including 50% overage for line priming, pump rate testing, and coating pan walls.
2. Prepare the coating suspension by slowly adding the Opadry II #85F18378 powder to the appropriate amount of DI water while continuously stirring the fluid with an overhead stirrer, ensuring sufficient wetting of the powder. Once all Opadry is added to the water, continue stirring at a low rpm for 60 minutes. The maximum hold time for the spray suspension is 24 hours.
3. Pre-coat the pan with Opadry by spraying the coating suspension for 5 to 10 minutes. After spraying dry the pan for 1 to 2 minutes.
4. Load the calculated amount of tablets in the coating pan.
5. Pre-heat the pan to the required bed temperature while jogging the pan. Calculate the tablet weight gain and confirm that the coating amount is between 2.5% and 3.5%. Stop spraying once that amount is sprayed. When coating amount is sufficient, dry the tablets for an additional 5 minutes. Turn the heating off and allow the tablets to cool while jogging the pan. When the bed temperature reaches 35° C. (±1° C.), the process is stopped. The coating pan door was remained closed during the cool down period.

### Example 7

### Tablets of Tromethamine Salt of Compound (1)

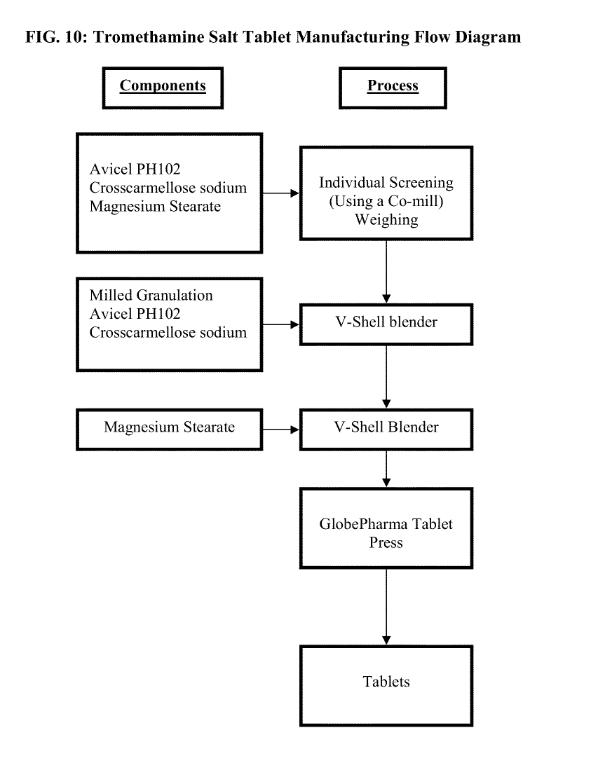

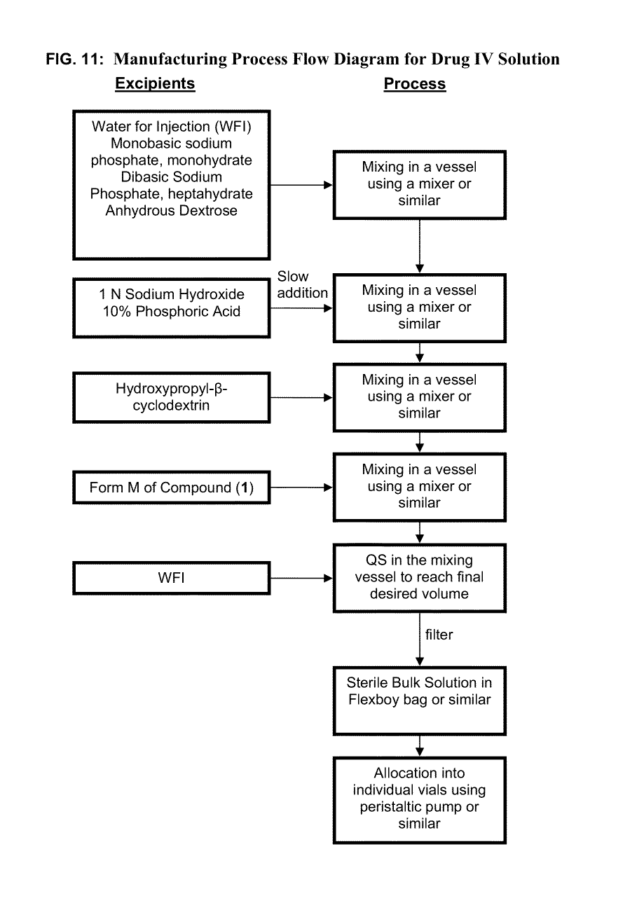

The formulation compositions for both the wet granulation and tablet blends of the active tablets are described in Tables 9a and 9b. The overall specification of the tromethamine salt tablets is described in Table 9c. A flow diagram for a wet granulation process and a manufacturing flow diagram for the tromethamine salt tablet are shown in FIGS. 9 and 10, respectively.

TABLE 9a
Tromethamine Salt of Compound (1) (250
mg) Wet granulation Composition

| | Amount | |
|---|---|---|
| | (mg) per | |
| Component | tablet | % W/W |
| Compound (1) (Tromethamine Salt) | 319.57 | 45.00 |
| Avicel PH-101 (microcrystalline | 221.50 | 31.19 |
| cellulose), NF, PhEur, JP | | |
| Lactose Monohydrate, #316, NF, PhEur, JP | 86.07 | 12.12 |
| Poloxamer 188 NF, PhEur, JP | 21.52 | 3.03 |
| Sodium Lauryl Sulfate NF, PhEur, JP | 12.29 | 1.73 |
| Povidone K12 USP | 30.75 | 4.33 |
| Ac-Di-Sol (cross carmellose sodium), NF, PhEur, JP | 18.46 | 2.60 |
| Total | 710.16 | 100.00 |

TABLE 9b
Tromethamine Salt of Compound (1) (250 mg) Tablet Composition

| | Amount | |
|---|---|---|
| | (mg) per | |
| Component | tablet | % W/W |
| Compound (1) Granulation (Milled) | 710.16 | 78.49 |
| Avicel PH-102 (microcrystalline | 135.7 | 15.00 |
| cellulose), NF, PhEur, JP | | |
| Lactose Monohydrate, #316, NF, PhEur, JP | 27.14 | 3.00 |
| Ac-Di-Sol (cross carmellose sodium), NF, PhEur, JP | 22.62 | 2.50 |
| Magnesium Stearate NF, PhEur, JP | 9.05 | 1.00 |
| Total | 904.67 | 99.99 |

TABLE 9c
Overall Composition of Tromethamine
Salt of Compound (1) (250 mg) Tablet

| | | % in dry | % in core |
|---|---|---|---|
| | | granule | tablet |
| intra | Compound (1) (Tromethamine salt) | 45.00 | 35.32 |
| granular | Avicel PH-101, NF, PhEur, JP | 31.19 | 24.48 |
| | Lactose Monohydrate, #316, NF, | 12.12 | 9.51 |
| | PhEur, JP | | |
| | Ac-Di-Sol, NF, PhEur, JP | 2.60 | 2.04 |
| | Sodium Lauryl Sulfate, NF, PhEur, JP | 1.73 | 1.36 |
| | Poloxamer 188, NF, PhEur, JP | 3.03 | 2.38 |
| | Povidone K12, USP | 4.33 | 3.40 |
| | Water, USP | na | na |
| | total granules: | 100.00 | 78.50 |
| extra | Avicel PH-101, NF, PhEur, JP | | 15.00 |
| granular | Lactose Monohydrate, #316, NF, | | 3.00 |
| | PhEur, JP | | |
| | Ac-Di-Sol, NF, PhEur, JP | | 2.50 |
| | Magnesium Stearate, NF, PhEur, JP | | 1.00 |
| | total core tablet: | | 100.00 |

The actual weights of each ingredient for the final tablet blend of the 250 mg tablet strength batch can be determined based on the yield calculations of the wet granulation (internal Phase). Sample calculation below

$$Weight of Excipient = \frac{\begin{matrix}Wet Granulation yield \% \times \\ Theorectical Weight of Excipient ( kg )\end{matrix}}{100}$$

### a) High Shear Wet Granulation Process Flow

1. An excess (10%) amount of Tromethamine salt of Compound (1), Avicel PH-101, Lactose Monohydrate, Poloxamer 188, Sodium Lauryl Sulfate, Povidone K12, and Cross Carmellose Sodium were weighed.
2. Using the Co-mill equipped with a #20 mesh screen (or hand screen), the excess amount of Compound (1), Avicel PH-101, Lactose Monohydrate, Poloxamer 188, Sodium Lauryl Sulfate, Povidone K12, and Cross Carmellose Sodium were screened at 70% speed.
3. The required amount of the sieved Compound (1), Avicel PH-101, Lactose Monohydrate, Poloxamer 188, Sodium Lauryl Sulfate, Povidone K12, and Cross Carmellose Sodium were weighed.
4. The materials from step 3 were transferred into a V-Shell blender.
5. The materials in the V-Shell blender was blended for 5 mins at the set speed (typically 25 RPM).
6. The contents of the V-Shell blender were emptied into LDPE bags (Bulk Wet Granulation blend).
7. The bulk wet granulation blend from step 6 was placed into a high shear granulator (Vector GMX.01) with a 1 L granulator bowl.
8. The blend was granulated. The wet granulation process was performed in two stages:
   Stage 1: 77% of the total amount of water required for the wet granulation was used to granulate the material at the prescribed process parameters. Once the water addition was complete, the granulation was stopped, and the walls, impeller, and chopper of the high shear granulator were scraped and the granulation was verified to determine if the visual endpoint was reached. If YES moved on to step 10, if NO proceeded to stage 2
   Stage 2: The remaining 23% of water was added and the material was granulated. Once the water addition was complete, the granulation was stopped, and the walls, impeller, and chopper of the high shear granulator were scraped and the granulation was verified to determine if the visual endpoint was reached.
   Stage 3: The material was granulated at the prescribed process parameters using just the impellor and chopper for ˜30 seconds. The granulation was stopped, and the walls, impeller, and chopper of the high shear granulator were scraped and the granulation was verified to determine if the visual endpoint was reached. If YES moved on to next step, if NO continued to granulate at the preceding process parameters (Stage 2) with 2 ml portions of water until the end-point was reached. Once the granulation end point was achieved, the material (Wet granulation blend) was screened through a #10 mesh screen and the screened material was transfered into a suitable container.
9. The material was then dried.
10. Once the material was confirmed dried, using a #20 (850 μm) mesh screen, hand all the dry granulations were creened.

### b) Tablet Manufacturing Process Flow

11. An excess (10%) amount of Avicel PH-102, Crosscarmellose Sodium, and Magnesium Stearate were weighed.
12. Using the Co-mill equipped with an 813 μm mesh screen (or hand screening), the excess amounts of Avicel PH-102, Crosscarmellose Sodium, and Magnesium Stearate were screened at 30% speed, and the sieved materials were placed in individual bags or containers.
13. The required amount of “sieved” Avicel PH-102, Crosscarmellose Sodium, Magnesium Stearate, and milled granulation were weighed.
14. The materials from previous step, except the magnesium stearate, was transferred into a V-Shell blender.
15. The materials in the V-Shell blender were blended for 5 mins at the set speed (typically 25 RPM).
16. Magnesium stearate was then added into the V-shell blender.
17. The resulting materials in the V-Shell blender were blended for 1 min at the set speed (typically 25 RPM).
18. The final blend was compressed using a GlobePharma tablet press according to the prescribed tablet compression process parameters. During the compression process, the individual and average tablet weights, hardness, thickness, and friability were monitored.
19. At the end of the run, all the tablets were dedusted and placed into bottles.

### Example 8

### IV Formulation of Compound (1)

A flow diagram for the manufacturing of drug intravenous solution is shown in FIG. 11. A description of the manufacturing process is provided below.

TABLE 10
Quantitative Batch Formula for Form M IV Solution

| Ingredient | mg/mL |
|---|---|
| Form M of Compound (1) | 5.00 |
| Hydroxypropyl-β-cyclodextrin, | 25.0 |
| HPβCD | |

70 mM Phosphate Buffer, pH 7.4, 12 L

| | |
|---|---|
| Sodium Phosphate, monobasic, | 2.182 |
| monohydrate | |
| Sodium Phosphate, dibasic, | 14.523 |
| heptahydrate | |
| Dextrose, Anhydrous | 25.0 |
| WFI | qs |

1. Sterilization of all the equipments and components to be used in the process was performed.
2. 10% Phosphoric Acid and 1M Sodium Hydroxide solution was prepared for pH adjustment
   a. 10% Phosphoric Acid (received as 86%):
      Approximately 250 mL of Water for Injection (WFI) was added to a 500 mL volumetric flask. Then 59 mL of phosphoric acid was slowly added to the flask. The mixture was then mixed.
   b. 1 M Sodium Hydroxide:
      Approximately 250 mL of WFI was added to a 500 mL volumetric flask. Then 20 g of Sodium Hydroxide was slowly added to the flask. The mixture was then mixed.
3. 70 mM phosphate buffer with dextrose was prepared −12 L
   a. The required quantities of dextrose, mono and dibasic sodium phosphate were weighed.
   b. Approximately 10 L of cool WFI (15-30° C.) was added to the compounding vessel.
   c. The mixture was then mixed.
   d. The weighed quantities of dextrose, mono and dibasic sodium phosphate, were added into the vessel. The mixture was then mixed until solution is clear.
   e. A 10 mL sample was taken for checking pH. If necessary, the pH was adjusted to have pH 7.4 (range: 7.2 to 7.6) with 10% Phosphoric Acid or 1 M Sodium Hydroxide Solution.
   f. QS to 12 L (12.2 kg, given the density of 1.013 g/mL) with WFI (15-30° C.). Mix for NLT 5 minutes.
4. Prepare Compound (1)/HPβCD solution
   a. The required quantities of HPβCD and Form M of Compound (1) were weighed.
   b. Approximately 9 kg of phosphate/dextrose buffer (15-30° C.) was added to compounding vessel with stir bar.
   c. The weighed HPβCD was added to the buffer solution and the mixture was stirred for NLT 5 minutes until the solution became clear.
   d. Compound (1) was then added into the compounding vessel. The vessel walls above the fluid were rinsed with 50-100 mL of buffer solution to wash down any residual drug that might be on the sides. The resulting mixture was then mixed for NLT 2 hours until the solution became clear.
   e. A 10 mL sample was taken and checked for pH. If necessary, the pH was adjusted to have pH 7.0 (range: 7.0 to 7.4) with 10% Phosphoric Acid or 1 M Sodium Hydroxide Solution.
   f. QS to 10 L (10.2 kg, given the density of 1.0218 g/mL) with phosphate/dextrose buffer (15-30° C.). Mix for NLT 5 minutes.
5. The bulk solution was filtered through 2, Millipak 200, 0.22 micron filters in series, into a sterile 20 L Flexboy bag using a peristaltic pump.
6. Using the Flexicon peristaltic filler, the solution was placed into vials. The filled vials were stored at 15-30° C.

### Example 9

### Dissolution Data

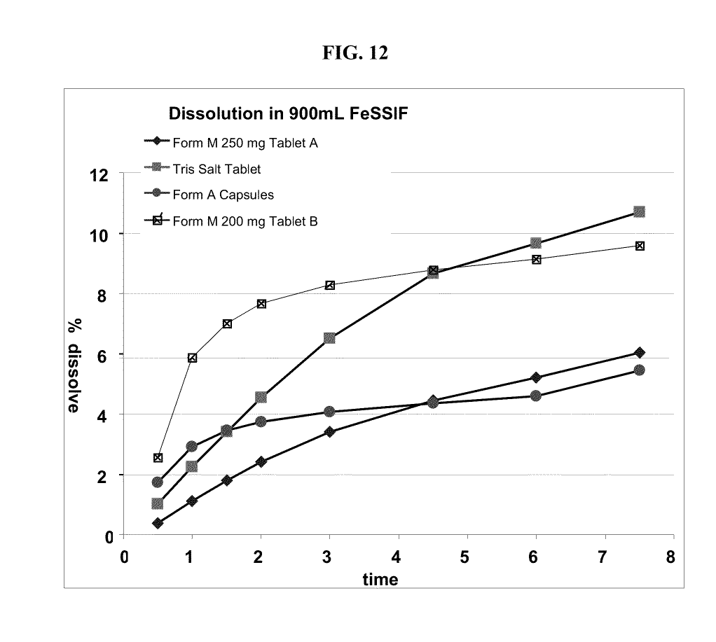

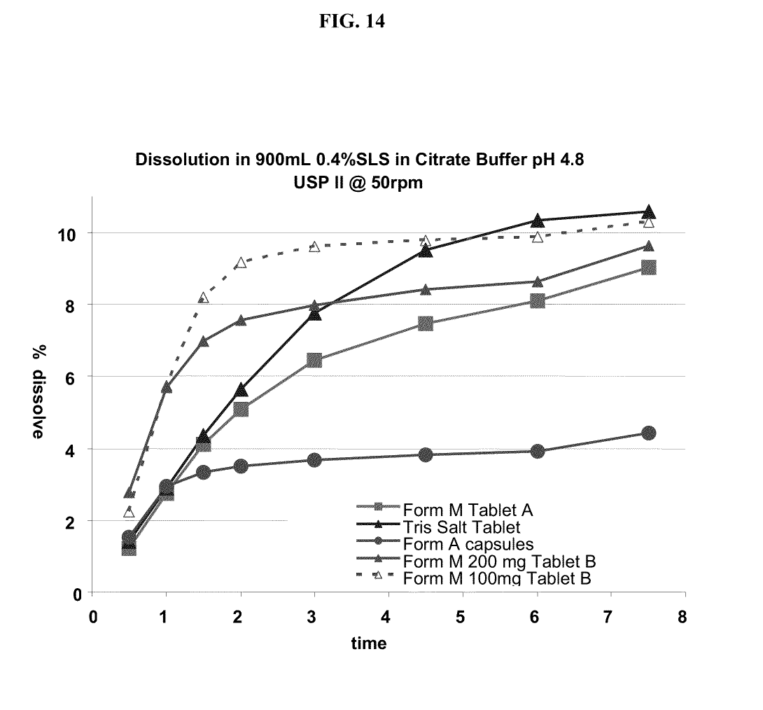

Dissolution profile of Form M tablets A, Form M tablets B, Form A capsules, and Tromethamine (Tris) salt tablets were obtained in FESSIF (Fed State Simulated Intestinal Fluid) (FIG. 12) and in 0.4% SLS (sodium lauryl sulfate) (FIG. 14):

### Dissolution in FESSIF:

Paddle: USP II

Paddle speed: 50 rpm

Volume: 900 mL

Media: FeSSIF

Temperature: 37° C.

### Dissolution in 0.4% SLS:

Paddle: USP II

Paddle speed: 50 rpm

Volume: 900 mL

Media: 0.4% SLS in citrate buffer pH 4.8

Temperature: 37° C.

### Example 10

### Simulation of Area Under the Curve (AUC) Ratio of Tablet B of Compound (1)

$$dissolution rate ( \frac{d M}{d t} ) = z \times {{M}_{0} ( \frac{{M}_{0} - M}{{M}_{0}} )}^{\frac{2}{3}} \times ( {C}_{s} - \frac{M}{V} )$$

TABLE 11
Simulated AUC Ratio of Tablet B (200 mg) of Compound (1)

AUC ratio

| | | | |
|---|---|---|---|
| In vitro Z in FESIF | In vivo Z | Upper limit | Lower limit |

| | | |
|---|---|---|
| 6.31E−02 | 1.48E−02 | control |

| | | | |
|---|---|---|---|
| 5.85E+00 | 1.48 | 1.1 | 1.08 |
| 5.89E−01 | 0.148 | 1.09 | 1.07 |
| 1.22E−01 | 0.0296 | 1.05 | 1.03 |
| 3.38E−02 | 0.0074 | 0.94 | 0.9 |
| 2.80E−02 | 5.92E−03 | 0.86 | 0.82 |
| 2.51E−02 | 5.18E−03 | 0.84 | 0.8 |
| 2.21E−02 | 4.44E−03 | 0.8 | 0.75 |
| 1.92E−02 | 3.70E−03 | 0.77 | 0.72 |

Each of the tablets and capsule included 200 mg of Compound (1).

An example of FESSIF that was used for obtaining the dissolution profile is shown in Table 12.

TABLE 12
Components of FESSIF

| Component | Amount per 1 L | |
|---|---|---|
| Sodium Hydroxide Pellets | 8.0 | g |
| Citric Acid Monohydrate | 16.1 | g |
| Sodium Chloride | 12.0 | g |
| Sodium Taurocholate | 8.1 | g |
| Lecithin (L-Alpha-Phosphatidyl Choline) | 3.1 | g |
| (Fisher Catelogue # 9 | | |

| | |
|---|---|
| Distilled Water | Qs to 1 L |

Dissolution rate of a formulation in a given medium is a fundamental property of a formulation. The in vitro dissolution rate can correlate with parameters derived from the observed in vivo pharmacokinetic data (e.g., AUC, Cₘₐₓ, etc.). For each specific drug, development of specific in vitro conditions is needed to establish an in vitro and in vivo correlation. It is found that an in vivo dissolution rate of Compound (1) in human is correlated to the dissolution rate of Compound (1) formulation in a simulated human intestinal fluid medium. The correlation was established based on a human plasma concentration-time profile of form M tablet of, form A capsule of, and tromethane salt tablet of Compound (1) and their dissolution rates in a simulated human intestinal medium. Both in vitro and in vivo dissolution profiles were expressed by the following equation:

$$\frac{d M}{d t} = z \times {{M}_{0} ( \frac{{M}_{0} - M}{{M}_{0}} )}^{\frac{2}{3}} \times ( {C}_{s} - \frac{M}{V} ) ,$$

where M is a dissolved mass of an active drug, M₀ is the initial mass of the active drug, t is the dissolution time, Cₛ is the solubility of the drug in the dissolution medium, V is the volume of dissolution medium, and z is the dissolution rate constant. z represents the mass transfer property of the drug in a given medium.

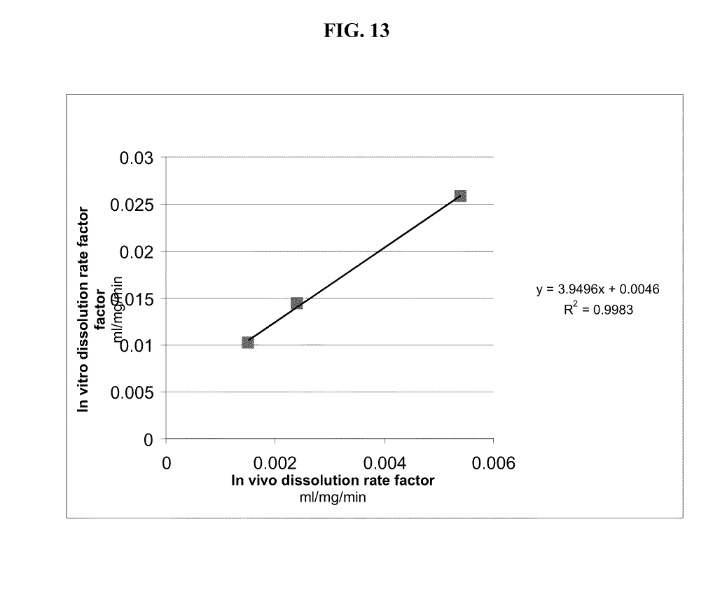

The in vivo z values of Compound (1) formulations in human were obtained using Gastroplus™ software by fitting the human plasma concentration-time profile. The in vitro z values were obtained by fitting the dissolution profile in a simulated human intestinal medium using Mathematica software. The correlation of in vitro z and in vivo z is shown in FIG. 13.

With the established correlation, the dissolution rate constant z of Compound (1) formulation in a simulated human intestinal medium is useful to predict the in vivo plasma concentration-time profile of Compound (1) formulations. Therefore, the z value determined in a simulated human intestinal medium is an important characteristic property of Compound (1) formulations relevant to in vivo performance.

A formulation of form M of Compound (1) showed a z value of 0.0631 ml/mg/min in a simulated human intestinal fluid. Solid formulations of Compound (1) with dissolution rate factor z values in the range of 0.025 ml/mg/min and 100.0 ml/mg/min in a simulated human intestinal fluid are expected to have an AUC ratio in the range of 0.8 to 1.1. (The upper limit of dissolution rate factor z is infinity when a solution is administered. The upper limit of the z value shown here represents the theoretical value of a spherical drug particle with molecular weight of 445.63 D and a radius of 1 nanometer and a diffusion layer thickness of 1 nanometer.) The AUC ratio is estimated based on a virtual trial simulation of 36 subjects in a crossover design. It is assumed that the CV % of inter-subject variability is 20% for clearance, volume of distribution, permeability and in vivo dissolution rate factor z. The intra-subject variability is ignored. The estimated AUC ratio with 90% confidence interval of formulations with hypothetical z values to the reference formulation is provided below in Table 13. Formulations with in vitro z values of 0.025-93.3 ml/mg/min are expected to meet the bioequivalence criteria to the reference formulation based on AUC ratio.

TABLE 13
The estimated AUC ratio with 90% confidence interval of formulations
with hypothetical z values to the reference formulation

| Summary | | AUC ratio to Phase 2b tablet | |
|---|---|---|---|
| In vitro Z factor | In vivo Z factor | Upper limit | Lower limit |
| 0.063 | 0.015 | reference | |
| 93.300 | 23.679 | 1.1 | 1.08 |
| 5.850 | 1.480 | 1.1 | 1.08 |
| 0.589 | 0.148 | 1.09 | 1.07 |
| 0.122 | 0.030 | 1.05 | 1.03 |
| 0.034 | 0.007 | 0.94 | 0.9 |
| 0.028 | 0.006 | 0.86 | 0.82 |
| 0.025 | 0.005 | 0.84 | 0.8 |
| 0.022 | 0.004 | 0.8 | 0.75 |
| 0.019 | 0.004 | 0.77 | 0.72 |

### Example 11

### Preparation of Additional Tablets Comprising Polymorphic Form M of Compound (1)

### A. Tablets C

Roller Compaction and Tablet Composition

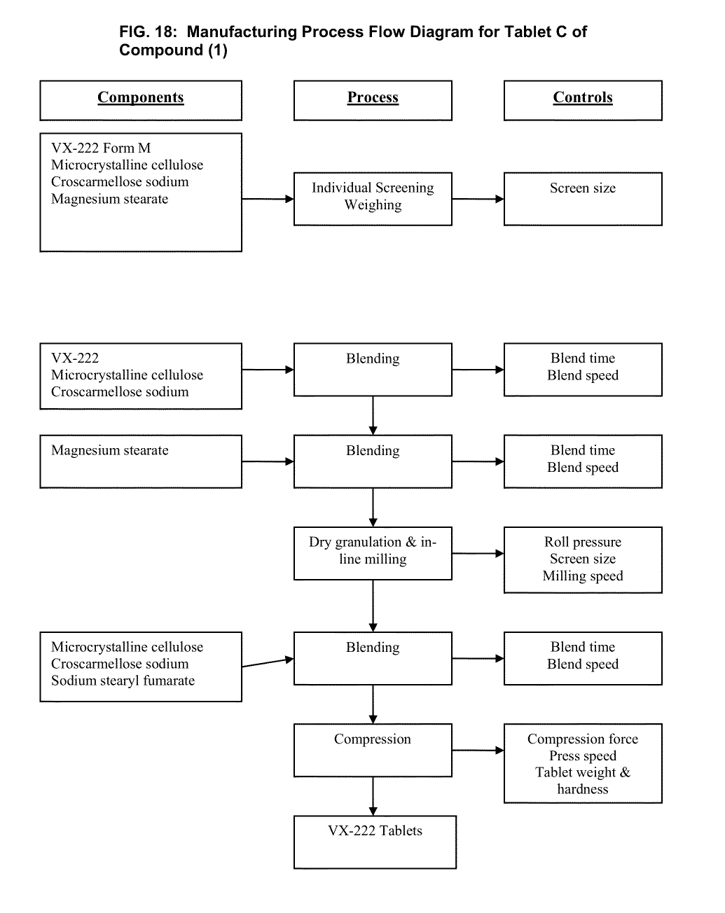

The overall composition specification of the tablets is described in Table 14. The tablet formulation was prepared in a similar manner as described above in Example 6 but using roller compaction instead of twin screw wet granulation process. The flow diagram for the manufacturing of Tablet C is shown in FIG. 18. In short, the manufacturing process includes:

Compound (1) (Form M), Microcrystalline cellulose, and croscarmellose sodium were individually screened, added to the blender and blended. Magnesium stearate was individually screened, added to the above blend and further blended. The blend was then dry granulated using a roller compactor and milled into granules. The granules were then further blended with individually screened Microcrystalline cellulose, croscarmellose sodium and sodium stearyl stearate. The final blend was then compressed into tablets. The final tablet contained 400 mg of Compound (1). Following the compression, SDD tablets were tested for release and packaged.

TABLE 18
Form M Tablet C Overall Composition
TSWG Granulation

| | Amount per |
|---|---|
| | Tablet (mg) |
| Form M of Compound (1) | 400 |
| Avicel PH-102 | 42.2 |
| Lactose Monohydrate | 0.0 |
| Ac-Di-Sol | 23.2 |
| Magnesium Stearate | 5.0 |
| total granules: | 470.4 |
| Avicel PH-101 | 192.1 |
| Ac-Di-Sol | 0.9 |
| Magnesium Stearate | 3.0 |
| Total | 666.4 |

### B. Tablets D

Wet Granulation and Tablet Composition

The tablet formulation was prepared in a similar manner, using Consigma 1 twin screw granulator with Fluid bed dryer, as described above in Example 6 for Tablet B. The overall Compound (1) granule composition tablet for HPC 2.25% is given in Table 19a and 19b.

TABLE 19a
Form M Tablet D Overall Composition
TSWG Granulation

| | Amount per | Amount in | Target |
|---|---|---|---|
| | Tablet (mg) | granulation | (g) |
| Form M of Compound (1) | 400 | 88.26 | 88.26 |
| Avicel PH-101 | 8.6 | 1.90 | 1.90 |
| Lactose Monohydrate | 11.2 | 2.47 | 2.47 |
| HPC-SL | 10.2 | 2.25 | 2.25 |
| Crosscarmellose Sodium | 23.2 | 5.12 | 5.12 |
| total granules: | 453.2 | 100.0 | 100.0 |

Tablet Blend

| | Amount per | Granulation | Target | Target |
|---|---|---|---|---|
| | Tablet (mg) | (%) | (%) | (g) |
| Form M of Compound | 453.2 | 100.0 | 62.32 | 21.81 |
| (1) Granulation | | | | |
| (Milled) | | | | |
| Avicel PH-101 | 237.8 | 52.8 | 32.69 | 11.44 |
| Sodium Stearyl | 21.8 | 4.45 | 3.00 | 1.05 |
| Fumarate | | | | |
| Crosscarmellose | 14.5 | 3.2 | 1.99 | 0.70 |
| Sodium | | | | |
| total granules: | 727.3 | 100.0 | 100.00 | 35.00 |

TABLE 19b
Other Overall Compositions Form M Tablet D

| | wt % | wt % | wt % | wt % | Amount | wt % in |
|---|---|---|---|---|---|---|
| | in pre- | in dry | in | in | in | coated |
| | granu- | granu- | core | coated | tablet | tablet in |
| | lation | lation | tablet | tablet | (mg) | ranges |
| Compound (1) | 90.29 | 88.04 | 55.00 | 53.40 | 400 | 50-60 |
| (Form M) | | | | | | |
| Avicel 101 | 1.94 | 1.89 | 1.18 | 1.15 | 8.6 | 1-2 |
| Lactose | 2.53 | 2.47 | 1.54 | 1.50 | 11.2 | 1-2 |
| Monohydrate | | | | | | |
| Cross- | 5.24 | 5.11 | 3.19 | 3.10 | 23.2 | 2-4 |
| carmellose | | | | | | |
| Sodium | | | | | | |
| | 100 | 97.50 | 60.91 | 59.14 | 443 | |
| HPC-SL | | 2.50 | 1.56 | 1.52 | 11.36 | 1-3 |
| Water | | 0 | 0 | 0 | 0 | |
| | 100 | 100 | 62.47 | 60.65 | 454.36 | |
| Avicel 101 | | | 32.53 | 31.58 | 236.56 | 25-35 |
| Cross- | | | 2.00 | 1.94 | 14.54 | 1-3 |
| carmellose | | | | | | |
| Sodium | | | | | | |
| Sodium Stearyl | | | 3.00 | 2.91 | 21.82 | 2-4 |
| Fumarate | | | | | | |
| | | | 100 | 97.09 | 727.27 | |
| Opadry II | | | | 2.91 | 21.82 | |
| Water | | | | 0 | 0 | |
| | | | | 100 | 749.09 | 100 |

The formulation composition and batch size for the pre granulation blend was given in Table 20a. Tables 20b, c, d, e, f and g gave the composition and batch size of the granulation binder solutions. The batch size of the binder solutions included a 100% overage for pump calibration and priming of solution lines.

TABLE 20a
Pre granulation composition and batch size

| | | Quantity |
|---|---|---|
| | % | per batch |
| Component | W/W | (kg) |
| Compound (1) crystalline (Form M) | 90.29 | 8.80 |
| Avicel PH-101 (microcrystalline | 1.94 | 0.19 |
| cellulose), NF, PhEur, JP | | |
| Lactose Monohydrate, #316, NF, PhEur, JP | 2.53 | 0.25 |
| Ac-Di-Sol (cross carmellose sodium), | 5.24 | 0.51 |
| NF, PhEur, JP | | |
| Total | 100.00 | 9.75 |

TABLE 20b
HPC (1.5%) Binder solution composition
and batch size (48% water)

| Component | % W/W | Batch size (g) |
|---|---|---|
| HPC-SL, USP | 3.03 | 30.3 |
| Water | 96.97 | 969.7 |
| Total | 100 | 1000 |

TABLE 20c
HPC (2.5%) Binder solution composition
and batch size (48% water)

| Component | % W/W | Batch size (g) |
|---|---|---|
| HPC-SL, USP | 4.95 | 49.5 |
| Water | 95.05 | 950.5 |
| Total | 100 | 1000 |

TABLE 20d
HPC (1.5%) Binder solution composition
and batch size (58% water)

| Component | % W/W | Batch size (g) |
|---|---|---|
| HPC-SL, USP | 2.52 | 25.2 |
| Water | 97.48 | 974.8 |
| Total | 100 | 1000 |

TABLE 20e
HPC (2.5%) Binder solution composition
and batch size (58% water)

| Component | % W/W | Batch size (g) |
|---|---|---|
| HPC-SL, USP | 4.13 | 41.3 |
| Water | 95.87 | 958.7 |
| Total | 100 | 1000 |

TABLE 20f
HPC (2.0%) Binder solution composition
and batch size (53% water)

| Component | % W/W | Batch size (kg) |
|---|---|---|
| HPC-SL, USP | 3.63 | 145.2 |
| Water | 96.37 | 3854.8 |
| Total | 100 | 4000 |

TABLE 20g
HPC (2.25%) Binder solution composition
and batch size (53% water)

| Component | % W/W | Batch size (kg) |
|---|---|---|
| HPC-SL, USP | 4.07 | 162.8 |
| Water | 95.93 | 3837.2 |
| Total | 100 | 4000 |

### a) Binder Solution Preparation (HPC 1.5%-2.5%)

The binder solution included the HPC binder. The solution was prepared based on 48, 53, and 58% w/w water content of the final dry granulation. An excess amount of 100% was prepared for pump calibration, priming lines, etc.
1. Weigh out the required amounts (Table 20b, c, d, e, f, and g) of HPC, and purified (DI) water.
2. Under constant stirring add the HPC-SL to the DI water and stir until fully dissolved. Turn down the stir rate such that only a partial vortex forms.
3. Stir the solution until all the solids present are visually fully dissolved.
4. Cover and let the solution sit for 2-4 hours until air bubbles in solution have disappeared. Alternatively, a partial vacuum can be pulled on the solution tank for up to an hour to degas the solution.

### b) Wet Granulation Process

1. Weigh the correct amounts of Compound (1), Croscarmellose Sodium, Avicel PH-101, and Lactose Monohydrate per Table 20a.
2. Using a U5 or U10 Comill equipped with a 32R screen and round impeller, delump the weighed out Compound (1), Lactose, and Avicel respectively at 4000 rpm in the U5, or 2800 rpm in the U10 into a bag or directly into the Bin blender.
3. Set up the blender and transfer the materials from step 2 into the blender if the material was delumped into a bag.
4. Blend the materials for 5 minutes at 23 RPM. Based on a bulk density of 0.4-0.5 g cc⁻¹, the blender should be 59%-74% full.
5. Take two×1.0 g samples, one for Karl Fischer (KF) and the other for LOD testing. These samples do not have to be taken with the sample thief
6. Charge 5 kg of the pre granulation blend into the loss in weight powder feeder directly from the blend shell. Empty the remainder of the blender contents into labeled LDPE bags or charge directly from the blend shell into the Loss in Weight feeder.
7. Set up the Consigma 1 twin screw granulator with the standard screw configuration as specified in Table 21.
8. Feed the dry blend into the extruder using the Barbender loss in weight feeder.
9. Inject the binder fluid into the granulator using the calibrated liquid pump.
10. Granulate the blend according to the prescribed experimental design shown in Table 22
11. Granulate approximately 4 kg of material for experiments 1-4 (1 kg per experiment), and approximately 6 kg of material for experiments 5 and 6 (3 kg per experiment)
12. The weight ratio of solution feed rate over powder feed rate varies from one experiment to the other (see the solution federates in Table 21 for all the experiments when the powder federates are kept constant at 167 g/min).
13. Collect the granules from each experiment into separate LDPE bags

### c) Fluid Bed Drying Process

14. Charge approximately 1 kg of granules into the fluid bed dryer and dry according to the parameters shown in Table 22.
15. Collect the dried granules into separate LDPE bags.

TABLE 21
Granulation Experiment design

| | | | | | Solution |
|---|---|---|---|---|---|
| | Water | HPC | | Granule | Feed Rate |
| Experiment | (%) | (%) | DOE | DL (%) | (g/min) |
| 1 | 48 | 1.50 | −− | 88.94 | 83.77 |
| 2 | 48 | 2.50 | −+ | 88.04 | 86.34 |
| 3 | 58 | 1.50 | +− | 88.94 | 100.76 |
| 4 | 58 | 2.50 | ++ | 88.04 | 103.44 |
| 5 | 53 | 2.00 | 00 | 88.49 | 93.56 |
| 6 | 53 | 2.25 | N/A | 88.26 | 94.22 |

TABLE 22
Process Control Parameters

| | |
|---|---|
| Parameter | Target Value and range |

Blending Operations

| | |
|---|---|
| Pre granulation | 252 (+/−10 revolutions) |
| blending | |

Twin Screw Wet Granulation

| | |
|---|---|
| Screw configuration | K/6,60\|XT\|6K/4,60\|1.5T\|6K/4,60\|1.5T\|2K/6,60 |
| Powder Feed Rate | 167.0 g/min (+/−0.5%) |
| Liquid Feed Rate | 83-103 g/min (+/−0.5%) |
| (0.8 mm nozzle) | |
| Screw Speed | 400 RPM (+/−10 RPM) |
| Barrel Temperature | 25° C. (range 20-30° C.) |

Granule Drying

| | |
|---|---|
| Inlet Air | 50° C. (range 45-55° C.) |
| Temperature | |
| Inlet Air flow | 30-100 m³/hr |
| Drying Time | 10-20 mins |

All references provided herein are incorporated herein in its entirety by reference. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A pharmaceutical composition comprising:
a) polymorphic form M or tromethamine salt of Compound (1) represented by the following structural formula: 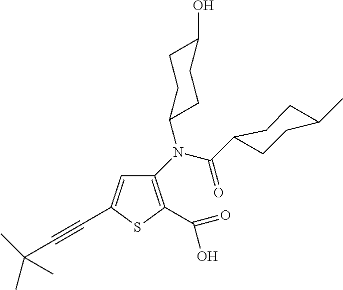 [SMILES: CC1CCC(C(=O)N(c2cc(C#CC(C)(C)C)sc2C(=O)O)C2CCC(O)CC2)CC1]
and
b) a filler.

2. The pharmaceutical composition of claim 1, wherein the composition comprises polymorphic form M of Compound (1).

3. The pharmaceutical composition of claim 1, wherein the composition includes:
25 wt % to 75 wt % of Compound (1) by the weight of the pharmaceutical composition; and
20 wt % to 75 wt % of the filler by the weight of the pharmaceutical composition.

4. (canceled)

5. (canceled)

6. The pharmaceutical composition of claim 1, wherein the filler includes a microcrystalline cellulose, a lactose, a sorbitol, a celluose, a calcium phosphate, a starch, or a sugar, or any combination thereof.

7. (canceled)

8. The pharmaceutical composition of claim 1, further including a disintegrant agent, wherein the composition includes 1 wt % to 15 wt % of the disintegrant agent by the weight of the composition.

9. (canceled)

10. The pharmaceutical composition of claim 9, wherein the disintegrant agent includes a croscarmellose, crospovidone and/or a metal starch glycolate.

11. (canceled)

12. The pharmaceutical composition of claim 9, further including a binder, wherein the binder comprises 0.5 wt % to 10 wt % of the weight of the pharmaceutical composition.

13. (canceled)

14. The pharmaceutical composition of claim 12, wherein the binder includes a polyvinyl pyrrolidone, a starch, a sugar, a microcrystalline cellulose, a hydroxy propyl methyl cellulose, a hydroxy propyl cellulose, and a hydroxy ethyl cellulose, and any combinations thereof.

15. The pharmaceutical composition of claim 12, further including a wetting agent in an amount of 0.25 wt % to 10 wt % of the weight of the pharmaceutical composition.

16. (canceled)

17. The pharmaceutical composition of claim 1, wherein the composition comprises:
a) 25 wt % to 70 wt % of polymorphic form M or tromethamine salt of Compound (1) by the weight of the pharmaceutical composition;
b) 1 wt % to 15 wt % of the disintegrant agent by the weight of the pharmaceutical composition; and
c) 25 wt % to 70 wt % of the filler by the weight of the pharmaceutical composition.

18. The pharmaceutical composition of claim 1, wherein the composition comprises:
a) 25 wt % to 70 wt % of polymorphic form M or tromethamine salt of Compound (1) by the weight of the pharmaceutical composition;
b) 0.5 wt % to 10 wt % of the binder by the weight of the pharmaceutical composition;
c) 1 wt % to 15 wt % of the disintegrant agent by the weight of the pharmaceutical composition; and
d) 25 wt % to 70 wt % of the filler by the weight of the pharmaceutical composition.

19. The pharmaceutical composition of claim 1, wherein the composition comprises:
a) 25 wt % to 60 wt % of polymorphic form M or tromethamine salt of Compound (1) by the weight of the pharmaceutical composition;
b) 0.25 wt % to 10 wt % of the wetting agent by the weight of the pharmaceutical composition;
c) 0.5 wt % to 10 wt % of the binder by the weight of the pharmaceutical composition;
d) 1 wt % to 15 wt % of the disintegrant agent by the weight of the pharmaceutical composition; and
e) 25 wt % to 70 wt % of the filler by the weight of the pharmaceutical composition.

20. (canceled)

21. (canceled)

22. (canceled)

23. The pharmaceutical composition of claim 1, wherein the composition comprises:
a) 25 wt % to 60 wt % of polymorphic form M or tromethamine salt of Compound (1) by the weight of the pharmaceutical composition;
b) 0.5 wt % to 10 wt % of a polyvinyl pyrrolidone by the weight of the pharmaceutical composition;
c) 0.25 wt % to 10 wt % of a copolymer of polyoxypropylene and polyoxyethylene by the weight of the pharmaceutical composition;
d) 0.25 wt % to 10 wt % of sodium lauryl sulfate by the weight of the pharmaceutical composition;
e) 25 wt % to 70 wt % of a microcrystalline cellulose by the weight of the pharmaceutical composition; and
f) 1 wt % to 15 wt % of croscarmellose sodium by the weight of the pharmaceutical composition.

24. The pharmaceutical composition of claim 1, wherein the composition comprises:
a) 25 wt % to 60 wt % of polymorphic form M or tromethamine salt of Compound (1) by the weight of the pharmaceutical composition;
b) 0.5 wt % to 10 wt % of a polyvinyl pyrrolidone by the weight of the pharmaceutical composition;
c) 0.25 wt % to 10 wt % of a copolymer of polyoxypropylene and polyoxyethylene by the weight of the pharmaceutical composition;
d) 0.25 wt % to 10 wt % of sodium lauryl sulfate by the weight of the pharmaceutical composition;
e) 25 wt % to 70 wt % of a microcrystalline cellulose by the weight of the pharmaceutical composition; and
f) 1 wt % to 15 wt % of croscarmellose sodium by the weight of the pharmaceutical composition.

24. The pharmaceutical composition of claim 1, wherein the composition comprises:
a) 25 wt % to 70 wt % of polymorphic form M or tromethamine salt of Compound (1) by the weight of the pharmaceutical composition;
b) 0.5 wt % to 10 wt % of a polyvinyl pyrrolidone by the weight of the pharmaceutical composition;
c) 0.25 wt % to 5 wt % of a copolymer of polyoxypropylene and polyoxyethylene by the weight of the pharmaceutical composition;
d) 0.25 wt % to 5 wt % of sodium lauryl sulfate by the weight of the pharmaceutical composition;
e) 0.25 wt % to 5 wt % of sodium stearyl fumarate by the weight of the pharmaceutical composition;
f) 20 wt % to 60 wt % of a microcrystalline cellulose by the weight of the pharmaceutical composition;
g) 0.5 wt % to 15 wt % of a lactose by the weight of the pharmaceutical composition; and
h) 1 wt % to 10 wt % of croscarmellose sodium by the weight of the pharmaceutical composition.

25. The pharmaceutical composition of claim 1, wherein the composition comprises:
a) 25 wt % to 70 wt % of polymorphic form M or tromethamine salt of Compound (1) by the weight of the pharmaceutical composition;
b) 0.5 wt % to 10 wt % of a hydroxylpropyl cellulose by the weight of the pharmaceutical composition;
c) 0.25 wt % to 10 wt % of sodium stearyl fumarate by the weight of the pharmaceutical composition;
d) 20 wt % to 60 wt % of a microcrystalline cellulose by the weight of the pharmaceutical composition; and
e) 0.5 wt % to 15 wt % of a lactose by the weight of the pharmaceutical composition; and
f) 1 wt % to 15 wt % of croscarmellose sodium by the weight of the pharmaceutical composition.

26. The pharmaceutical composition of claim 1, wherein the composition comprises:
a) 25 wt % to 70 wt % of polymorphic form M or tromethamine salt of Compound (1) by the weight of the pharmaceutical composition;
b) 0.25 wt % to 10 wt % of magnesium stearate by the weight of the pharmaceutical composition;
c) 25 wt % to 70 wt % of a microcrystalline cellulose by the weight of the pharmaceutical composition; and
d) 1 wt % to 15 wt % of croscarmellose sodium by the weight of the pharmaceutical composition.

27. The pharmaceutical composition of claim 1, further including a glidant selected from the group consisting of an amorphous silicon dioxide and talc.

28. The pharmaceutical composition of claim 1, wherein a dissolution rate factor z of the composition is at least about 0.025 ml/mg/min.

29. (canceled)

30. (canceled)

31. The pharmaceutical composition of claim 1, wherein the composition comprises:
a) 25 wt % to 70 wt % of polymorphic form M of Compound (1) by the weight of the pharmaceutical composition; and
b) 25 wt % to 70 wt % of a filler by the weight of the pharmaceutical composition, the filler being selected from the group consisting of a microcrystalline cellulose, a lactose, a sorbitol, a celluose, a calcium phosphate, a starch, and a sugar, and any combination thereof, wherein the formulation is in a tablet or capsule form.

32. A pharmaceutical composition comprising polymorphic form M of Compound (1) by weight of the composition, wherein Compound (1) is represented by the following structural formula: 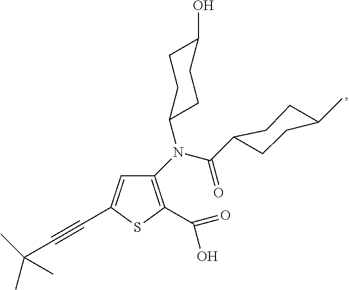 [SMILES: CC1CCC(C(=O)N(c2cc(C#CC(C)(C)C)sc2C(=O)O)C2CCC(O)CC2)CC1]
wherein the composition has a dissolution rate represented by the following equation:
$$dissolution rate ( \frac{d M}{d t} ) = z \times {{M}_{0} ( \frac{{M}_{0} - M}{{M}_{0}} )}^{\frac{2}{3}} \times ( {C}_{s} - \frac{M}{V} )$$
wherein M is a dissolved mass of Compound (1), M₀ is an initial mass of Compound (1), t is a dissolution time, Cₛ is a solubility of Compound (1) in a fed state simulated intestinal fluid, V is volume of the fed state simulated intestinal fluid, and z is a dissolution rate factor, wherein the composition has a z value greater than 0.025 ml/mg/minute.

33. (canceled)

34. (canceled)

35. (canceled)

36. (canceled)

37. A pharmaceutical composition comprising:
a) polymorphic form M or tromethamine salt of Compound (1) represented by the following structural formula: 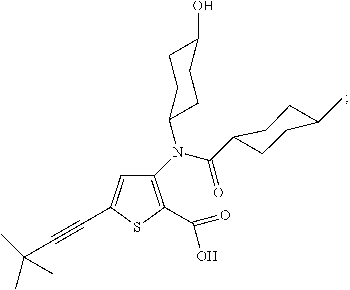 [SMILES: CC1CCC(C(=O)N(c2cc(C#CC(C)(C)C)sc2C(=O)O)C2CCC(O)CC2)CC1]
b) a complexing agent; and
c) a buffering agent

38. The pharmaceutical composition of claim 37, wherein the composition comprise polymorphic form M of Compound (1).

39. The pharmaceutical composition of claim 38, wherein the composition includes:
1 mg/mL to 20 mg/mL of Compound (1);
1 wt % to 25 wt % of the complexing agent by weight of the pharmaceutical composition; and
0.01 M to 0.1 M of the buffering agent.

40. The pharmaceutical composition of claim 39, wherein the complexing agent includes a cyclodextrin.

41. (canceled)

42. (canceled)

43. (canceled)

44. (canceled)

45. (canceled)

46. (canceled)

47. (canceled)

48. (canceled)

49. (canceled)

50. A method of preparing a pharmaceutical composition, comprising:
providing a mixture that includes a polymorphic form M or tromethamine salt of Compound (1) and a filler to form the pharmaceutical composition, wherein Compound (1) is represented by the following structural formula:

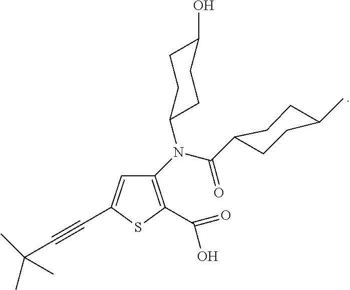 [SMILES: CC1CCC(C(=O)N(c2cc(C#CC(C)(C)C)sc2C(=O)O)C2CCC(O)CC2)CC1]

51. The method of claim 50, wherein the providing said mixture of Compound (1) and filler includes:
providing granules of Compound (1) that include: i) 35 wt % to 95 wt % of a polymorphic form M or tromethamine salt of Compound (1); and ii) an intra-granular excipient that includes 3 wt % to 60 wt % of a filler, by the weight of the granules; and
mixing the granules of Compound (1) with extra-granular excipients that include 10 wt % to 50 wt % of a filler by the weight of the pharmaceutical composition.

52. (canceled)

53. (canceled)

54. (canceled)

55. The method of claim 51, wherein the providing said mixture of Compound (1) and the filler includes:
providing granules of Compound (1) that include a polymorphic form M or tromethamine salt of Compound (1), a wetting agent, a binder, and intra-granular excipients that include a filler and a disintegrant agent; and
mixing the granules of Compound (1) with extra-granular excipients that include a disintegrant agent and a filler.

56. The method of claim 55, wherein the intragranular excipients include 3 wt % to 50 wt % of a filler and 0.5 wt % to 5 wt % of a disintegrant agent, by the weight of the granules, and the extra-granular excipients include 15 wt % to 50 wt % of a filler and 0.5 wt % to 10 wt % of a disintegrant agent, by the weight of the pharmaceutical composition.

57. The method of claim 56, wherein the providing granules of Compound (1) includes:
providing a binder solution that includes the binder and the wetting agent;
providing a pre-granulation composition that includes polymorphic form M or tromethamine salt of Compound (1) and the intra-granular excipients;
mixing the binder solution and the pre-granulation composition to form the granules of Compound (1).

58. The method of 57, wherein the mixing of the binder solution and the pre-granulation composition includes feeding the pre-granulation composition into a twin screw extruder and introducing the binder solution into the twin screw extruder.

59. The method of claim 58, wherein the binder solution includes 0.5 wt % to 10 wt % of a binder by the weight of the pharmaceutical composition.

60. The method of claim 59, wherein the binder solution further includes 0.25 wt % to 10 wt % of a wetting agent by the weight of the pharmaceutical composition.

61. The method of claim 59, wherein the binder solution further includes water in a range of 5 wt % to 60 wt % by the weight of the pharmaceutical composition.

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. The method of claim 50, further comprising compressing the pharmaceutical composition of Compound (1) into a tablet.

74. A method of inhibiting or reducing the activity of HCV polymerase in a biological in vitro sample, comprising administering to the sample an effective amount of a pharmaceutical composition according to any one of claim 1.

75. A method of treating a HCV infection in a subject, comprising administering to the subject a therapeutically effective amount of a pharmaceutical composition according to any one of claim 1.

76. A method of inhibiting or reducing the activity of HCV polymerase in a subject, comprising administering to the subject a therapeutically effective amount of a pharmaceutical composition according to any one of claim 1.

77-89. (canceled)
